(19) United States
(12) Patent Application Publication
(10) Pub. No.: US 2023/0116114 A1
(43) Pub. Date: Apr. 13, 2023

(54) COMPOUNDS AND COMPOSITIONS FOR TREATING CNS DISORDERS

(71) Applicant: Blue Oak Pharmaceuticals, Inc., Concord, MA (US)

(72) Inventors: Kerry L. Spear, Concord, MA (US); Douglas Burdi, Arlington, MA (US)

(21) Appl. No.: 17/790,525

(22) PCT Filed: Dec. 29, 2020
(86) PCT No.: PCT/US2020/067298

§ 371 Date: Jul. 1, 2022

Related U.S. Application Data
(60) Provisional application No. 62/956,895, filed on Jan. 3, 2020.
(60) Provisional application No. 63/128,375, filed on Dec. 21, 2020.

(51) Int. Cl. C07D 487/08; C07D 491/18

(52) U.S. Cl. CPC C07D 487/08; C07D 491/18

(57) ABSTRACT

The present disclosure provides compounds and pharmaceutical compositions thereof. Methods of making and using the compounds are also provided. The compounds can be used for the treatment, prevention, diagnosis and/or management of various CNS disorders.

### CROSS-REFERENCE TO RELATED APPLICATIONS

This application claims priority to U.S. Prov. Application No. 62/956,895 filed Jan. 3, 2020, and U.S. Prov. Application No. 63/128,375 filed Dec. 21, 2020, the contents of each of which are incorporated herein by reference in their entirety.

### BACKGROUND

The central nervous system (CNS) has been shown to influence a variety of physiologic functions and is involved in a variety of diseases (or disorders), such as neurological disorders and psychiatric disorders. Non-limiting CNS disorders include depression, anxiety, schizophrenia, bipolar disorder, obsessive compulsive disorder (OCD), panic disorder, and posttraumatic stress disorder (PTSD). These disorders affect a person's thoughts, mood, behavior and social interactions and can significantly impair daily functioning. Significant medical, social and economic burdens are associated with these diseases. However, these diseases are usually complex in nature and involve multiple neuronal circuits. Traditional target-based approaches are not efficient in discovering meaningful treatments. There remains a need to find therapeutic agents, methods and therapies for the treatment of CNS diseases.

### SUMMARY OF THE DISCLOSURE

The present disclosure provides compounds and compositions for treating CNS disorders such as psychiatric and neurological disorders and diseases.

In some embodiments, the present disclosure provides a compound of Formula (I):

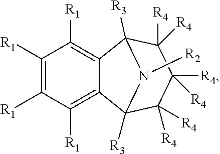

or a pharmaceutically acceptable salt thereof, methods of preparing the compounds, and methods of using the compounds, wherein
R1 is independently H, optionally substituted alkyl, optionally substituted cycloalkyl, halogen, hydroxyl, alkoxyl, ether, CN, amine, aryl, or heteroaryl; wherein optionally any two adjacent R1 groups, together with the carbons to which they are attached, form a 5 to 8-membered carbocycle or heterocycle;
R2 is H, optionally substituted lower alkyl, optionally substituted cycloalkyl, heterocyclic, or multicyclic group; wherein optionally R2 and any R4, together with the nitrogen and carbon to which they are attached, form a 5 to 8-membered heterocycle;
R3 is independently H, halogen, optionally substituted lower alkyl or cycloalkyl; wherein optionally R3 and any adjacent R4, together with the carbons to which they are attached, form a 5 to 8-membered carbocycle or heterocycle; and
R4 is independently H, optionally substituted lower alkyl, optionally substituted cycloalkyl, halogen, alkoxyl, CN, amine, aryl, heteroaryl, or carbonyl; wherein optionally any two geminal R4 groups, together with the carbon to which they are attached, form a 3 to 8-membered carbocycle or heterocycle.

In some embodiments, the present disclosure provides a compound of Formula (II):

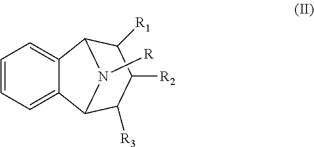

or a pharmaceutically acceptable salt thereof, methods of preparing the compounds, and methods of using the compounds, wherein
R is hydrogen, an optionally substituted alkyl group, an optionally substituted cycloalkyl group, an optionally substituted heterocyclic group, or an optionally substituted multicyclic group; and
R1, R2, or R3, independently, is hydrogen, an optionally substituted alkyl group, an optionally substituted cycloalkyl group, a hydroxyl group, an alkoxy group, an aryloxy group, an amino group, or a halogen.

In some embodiments, the present disclosure provides a compound of Formula (III):

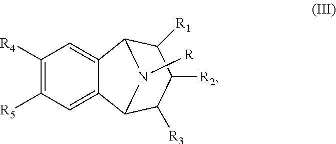

or a pharmaceutically acceptable salt thereof, methods of preparing the compounds, and methods of using the compounds, wherein
R is hydrogen, an optionally substituted alkyl group, an optionally substituted cycloalkyl group, a heterocyclic group, or a multicyclic group;
R1, R2, or R3, independently, is hydrogen, an optionally substituted alkyl group, an optionally substituted cycloalkyl group, a hydroxyl group, an alkoxy group, an aryloxy group, an amino group, or a halogen; and
R4 or R5, independently, is hydrogen, optionally substituted lower alkyl (including but not limited to haloalkyl, alkoxy, amino alkyl, arylalkyl, or heteroarylalkyl), optionally substituted cycloalkyl, halogen, hydroxyl, alkoxyl, ether, CN, amine, aryl, or heteroaryl; wherein any two adjacent R1 groups, together with the carbons to which they are attached, may form a 5 to 8-membered carbocycle or heterocycle which may be optionally substituted.

The present disclosure also provides Compounds 1-49, or a pharmaceutically acceptable salt thereof.

The present disclosure also provides a pharmaceutical composition comprising the compounds of the present disclosure or a pharmaceutically acceptable salt thereof, and at least one pharmaceutically acceptable excipient.

The present disclosure further provides a method of treating, preventing, or managing a CNS disorder in a subject in need thereof, comprising administering to said subject an effective amount of the compounds of the present disclosure or an effective amount of the pharmaceutical composition comprising the compounds of the present disclosure or a pharmaceutically acceptable salt thereof. In some cases, the CNS disorder is a neurological or psychiatric disorder. In some cases, the CNS disorder is depression, anxiety, cognitive impairment, psychosis, schizophrenia, bipolar disorder, obsessive compulsive disorder (OCD), panic disorder, posttraumatic stress disorder (PTSD), addiction, social disorder, attention deficit hyperactivity disorder (ADHD), or autism. In some cases, the CNS disorder is depression. In some cases, the CNS disorder is bipolar depression, unipolar depression, major depressive disorder, treatment-resistant depression, suicidal behavior disorder, or anhedonia.

### DETAILED DESCRIPTION

CNS drug discovery differs from most other therapeutic areas because of the complex and multigenic nature of most psychiatric and neurological disorders. Of particular interest are therapies that (i) have a rapid onset of action to treat the CNS disorder, (ii) have efficacy in achieving and sustaining long term remission, (iii) have improved safety and a more tolerable side effect profile.

### I. Compositions of the Disclosure

Applicant has used multiple and complementary assays (chemical and behavioral) to drive therapeutic drug discovery.

The compounds were evaluated for their CNS related properties (e.g., the treatment, prevention or diagnosis of CNS or CNS-related disorders and/or amelioration of symptoms) using neuropharmacological screening methods described in S. L. Roberts et al., Front. Neurosci. 2011, 5:103 (hereinafter referred to as “Roberts,” the contents of which are incorporated herein by reference in their entirety).

According to Roberts, intact systems are useful in detecting improvement in disease-relevant endpoints, because psychiatric diseases generally result from disorders of cell-cell communication or circuitry. The endpoints are typically behavioral in nature, often requiring human observation and interpretation. In order to test multiple compounds for behavioral effects relevant to neurological and psychiatric diseases and disorders, PsychoGenics, Inc. (Paramus, N.J. “PGI”) developed SmartCube®, an automated system in which behaviors of compound-treated mice are captured by digital video and analyzed with computer algorithms to facilitate testing of multiple compounds for behavioral effects relevant to psychiatric disease. (D. Brunner et al., Drug Discov. Today 2002, 7:S107-S112; Alexandrov et al., Eur. J. Pharmacol. 2015, 753:127-134; the contents of each of which are incorporated herein by reference in their entirety).

The SmartCube® system is described in Example 2.2 of U.S. Pat. No. 9,758,529 and Example 289 of WIPO Publication No. WO2018/023070, the contents of each of which are incorporated herein by reference in their entirety.

According to the present disclosure, compounds identified may be evaluated using the SmartCube® system. The results of such analysis correlate certain patterns or combinations of behaviors to CNS effects along a percent probability scale of 0-100 where zero (0) reflects the limit of quantitation (LOQ).

Other ranges include (A) less than 5% (which includes values of 1, 2, 3, and 4%, inclusive of intermediate values in 0.1 increments up to 4.9%); (B) from equal to 5% to less than 25% (which includes values of 5, 6, 7, 8, 9, 10, 11, 12, 13, 14, 15, 16, 17, 18, 19, 20, 21, 22, 23, and 24% inclusive of intermediate values in 0.1 increments up to 24.9%); (C) from equal to 25% to less than 50% (which includes values of 25, 26, 27, 28, 29, 30, 31, 32, 33, 34, 35, 36, 37, 38, 39, 40, 41, 42, 43, 44, 45, 46, 47, 48 and 49 inclusive of intermediate values in 0.1 increments up to 49.9%); and (D) from equal to 50% to less than or equal to 100% (which includes values of 50, 51, 52, 53, 54, 55, 56, 57, 58, 49, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, and 99 inclusive of intermediate values in 0.1 increments up to 99.9%).

In general, the compounds of the present disclosure are bridged bicyclic or multicyclic small molecule compounds described below.

In some embodiments, the molecular weight (MW) of the compound may not be more than 500 g/mol. In some embodiments, the molecular weight (MW) of the compound may not be more than 300 g/mol. In some embodiments, the compound has low lipophilicity. For example, the logP of the compound may not be more than 3. In some embodiments, the hydrogen bond donor (HBD) of the compound may not be more than 3. In some embodiments, the hydrogen bond acceptor (HBA) of the compound may not be more than 3.

Unless otherwise stated, structures presented herein can include all stereochemical forms of the structure; i.e., the R and S configurations for each asymmetric center. Therefore, single stereochemical isomers as well as enantiomeric and diastereomeric mixtures of the present compounds are within the scope of the present disclosure. Compounds of the present disclosure may exist in alternative tautomeric forms. A representation of one tautomer is meant to include the other.

Unless otherwise stated, structures presented herein are also meant to include compounds which differ only in the presence of one or more isotopically enriched atoms. For example, replacements of a hydrogen atom by deuterium or tritium, or carbon atom by a ¹³C- or ¹⁴C-enriched carbon are within the scope of the present disclosure.

Generic Structure (I):

In some embodiments, the compounds of the present disclosure have a general structure of Formula (I):

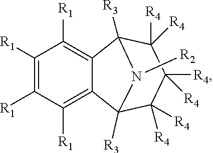

or a pharmaceutically acceptable salt thereof,

wherein R1 is independently H, optionally substituted alkyl (including but not limited to lower alkyl, haloalkyl, alkoxy, amino alkyl, arylalkyl, or heteroarylalkyl), optionally substituted cycloalkyl (such as a 3-8 membered cycloalkyl), halogen, hydroxyl, alkoxyl, ether, CN, amine, aryl, or heteroaryl; wherein any two adjacent R1 groups, together with the carbons to which they are attached, may form a 5 to 8-membered carbocycle or heterocycle which may be optionally substituted;

R2 is H, optionally substituted lower alkyl (including but not limited to heterocycloalkyl, haloalkyl, alkoxy, amino alkyl, arylalkyl, or heteroarylalkyl), optionally substituted cycloalkyl (such as a 3-8 membered cycloalkyl), an optionally substituted heterocyclic group, or an optionally substituted multicyclic group; wherein optionally R2 and any R4, together with the nitrogen and carbon to which they are attached, may form a 5 to 8-membered heterocycle which may be optionally substituted,

R3 is independently H, halogen (such as F), optionally substituted lower alkyl or optionally substituted cycloalkyl; wherein optionally R3 and any adjacent R4, together with the carbons to which they are attached, may form a 5 to 8-membered carbocycle or heterocycle which may be optionally substituted; and

R4, can independently be H, optionally substituted lower alkyl (including but not limited to haloalkyl, alkoxy, amino alkyl, arylalkyl, heteroarylalkyl, or heterocycloalkyl), optionally substituted cycloalkyl, halogen (such as F), alkoxyl, CN, amine, aryl, heteroaryl, or carbonyl; wherein optionally any two geminal R4 groups, together with the carbon to which they are attached, may form a 3 to 8-membered carbocycle or heterocycle which may be optionally substituted.

In some embodiments, any two adjacent R1 groups, together with the carbons to which they are attached, may form a 5 to 8-membered aromatic carbocycle or aromatic heterocycle which may be optionally substituted. In some embodiments, any two adjacent R1 groups, together with the carbons to which they are attached, may form a 5 to 8-membered non-aromatic carbocycle or non-aromatic heterocycle which may be optionally substituted.

In some embodiments, R2 is an alkyl group. In some embodiments, the alkyl group may be substituted with at least one functional group, such as a hydroxyl group, an alkoxyl group, an aryloxy group, an amino group, an aryl group, or a halogen. In some embodiments, there is a substituent (such as but not limited to a methyl group) at the alpha position of the alkyl group. In some embodiments, the alkyl group is a lower alkyl group.

In some embodiments, R2 is a cyclic alkyl group, such as 3-8 membered cycloalkyl. In some embodiments, R2 is a heterocyclic group.

In some embodiments, R2 and any R4, together with the nitrogen and carbon to which they are attached, may form a 5 to 8-membered non-aromatic heterocycle which may be optionally substituted.

In some embodiments, R2 is a multicyclic group. A multicyclic group, as used herein, refers a chemical group wherein at least 2 cyclic groups are fused together. The cyclic groups may be aromatic or non-aromatic. The cyclic groups may comprise a heteroatom such as O, N, or S. In some embodiments, R2 is a bicyclic group. In some embodiments, R2 comprises an aromatic carbocycle fused to a non-aromatic carbocycle. In some embodiments, R2 comprises an aromatic heterocycle fused to a non-aromatic heterocycle.

In some embodiments, R3 and any R4, together with the carbons to which they are attached, may form a 5 to 8-membered non-aromatic carbocycle or heterocycle which may be optionally substituted.

In some embodiments, the two adjacent R4 groups, together with the carbon(s) to which they are attached, form a 5 to 8-membered aromatic carbocycle or heterocycle which may be optionally substituted. In some embodiments, the two adjacent R4 groups, together with the carbon(s) to which they are attached, form a 3 to 8-membered non-aromatic carbocycle or heterocycle which may be optionally substituted.

Non-limiting examples of compounds encompassed by Formula (I) include Compounds 1-38 and 40-49 or a pharmaceutically acceptable salt thereof:

TABLE 1
Non-limiting examples of compounds
encompassed by Formula (I)

| Compound No. | Structures |
|---|---|
| 1 | 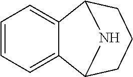 [SMILES: c1ccc2c(c1)C1CCCC2N1] |
| 2 | 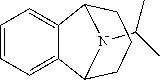 |
| 3 | 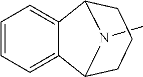 |
| 4 | 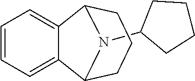 [SMILES: c1ccc2c(c1)C1CCCC2N1C1CCCC1] |
| 5 | 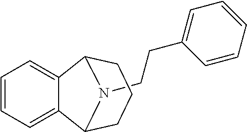 [SMILES: c1ccc(CCN2C3CCCC2c2ccccc23)cc1] |
| 6 | 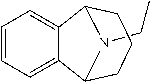 |
| 7 | 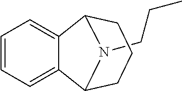 |
| 8 | 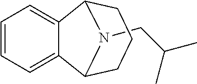 [SMILES: CC(C)CN1C2CCCC1c1ccccc12] |
| 9 | 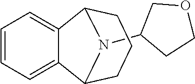 [SMILES: c1ccc2c(c1)C1CCCC2N1C1CCOC1] |
| 10 | 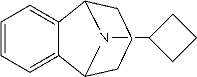 [SMILES: c1ccc2c(c1)C1CCCC2N1C1CCC1] |
| 11 | 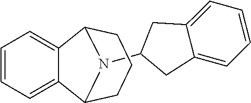 [SMILES: c1ccc2c(c1)CC(N1C3CCCC1c1ccccc13)C2] |
| 12 | 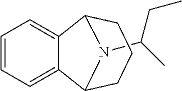 [SMILES: CCC(C)N1C2CCCC1c1ccccc12] |
| 13 | 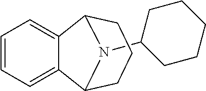 [SMILES: c1ccc2c(c1)C1CCCC2N1C1CCCCC1] |
| 14 | 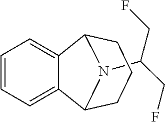 [SMILES: FCC(CF)N1C2CCCC1c1ccccc12] |
| 15 | 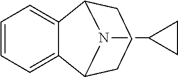 [SMILES: c1ccc2c(c1)C1CCCC2N1C1CC1] |
| 16 | 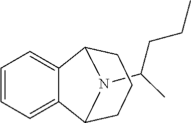 [SMILES: CCCC(C)N1C2CCCC1c1ccccc12] |
| 17 | 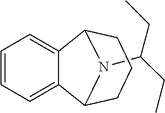 [SMILES: CCC(CC)N1C2CCCC1c1ccccc12] |
| 18 | 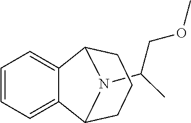 [SMILES: COCC(C)N1C2CCCC1c1ccccc12] |
| 19 | 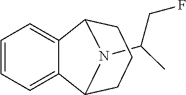 [SMILES: CC(CF)N1C2CCCC1c1ccccc12] |
| 20 | 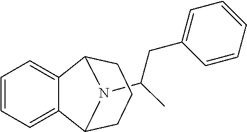 [SMILES: CC(Cc1ccccc1)N1C2CCCC1c1ccccc12] |
| 21 | 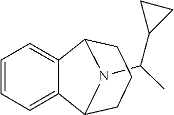 [SMILES: CC(C1CC1)N1C2CCCC1c1ccccc12] |
| 22 | 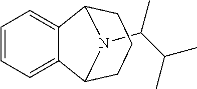 [SMILES: CC(C)C(C)N1C2CCCC1c1ccccc12] |
| 23 | 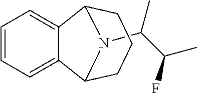 [SMILES: CC([C@@H](C)F)N1C2CCCC1c1ccccc12] |
| 24 | 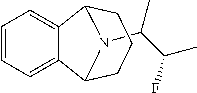 [SMILES: CC([C@H](C)F)N1C2CCCC1c1ccccc12] |
| 25 | 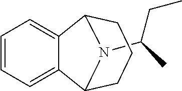 [SMILES: CC[C@@H](C)N1C2CCCC1c1ccccc12] |
| 26 | 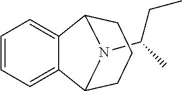 [SMILES: CC[C@H](C)N1C2CCCC1c1ccccc12] |
| 27 | 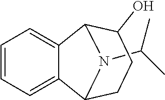 [SMILES: CC(C)N1C2CCC(O)C1c1ccccc12] |
| 28 | 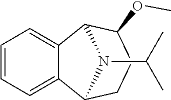 |
| 29 | 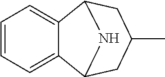 |
| 30 | 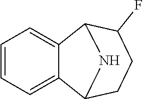 [SMILES: FC1CCC2NC1c1ccccc12] |
| 31 | 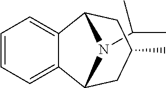 [SMILES: CC(C)N1[C@@H]2C[C@@H](C)C[C@H]1c1ccccc12] |
| 32 | 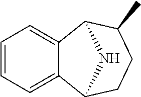 |
| 34 | 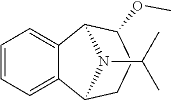 [SMILES: CO[C@@H]1CC[C@H]2c3ccccc3[C@@H]1N2C(C)C] |
| 35 | 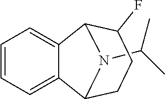 [SMILES: CC(C)N1C2CCC(F)C1c1ccccc12] |
| 36 | 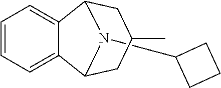 [SMILES: CC1CC2c3ccccc3C(C1)N2C1CCC1] |
| 37 | 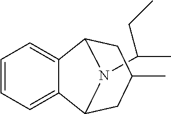 [SMILES: CCC(C)N1C2CC(C)CC1c1ccccc12] |
| 38 | 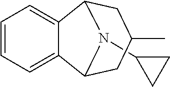 [SMILES: CC1CC2c3ccccc3C(C1)N2C1CC1] |
| 39 | 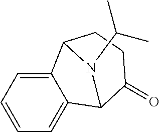 [SMILES: CC(C)N1C2CCC(=O)C1c1ccccc12] |
| 40 | 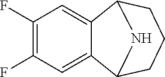 [SMILES: Fc1cc2c(cc1F)C1CCCC2N1] |
| 41 | 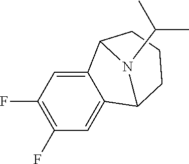 [SMILES: CC(C)N1C2CCCC1c1cc(F)c(F)cc12] |
| 42 | 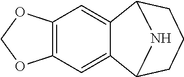 [SMILES: c1c2c(cc3c1C1CCCC3N1)OCO2] |
| 43 | 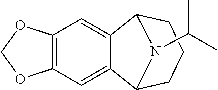 [SMILES: CC(C)N1C2CCCC1c1cc3c(cc12)OCO3] |
| 44 | 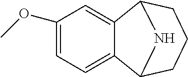 |
| 45 | 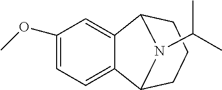 [SMILES: COc1ccc2c(c1)C1CCCC2N1C(C)C] |
| 46 | 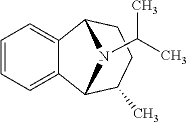 [SMILES: CC(C)N1[C@H]2CC[C@H](C)[C@@H]1c1ccccc12] |
| 47 | 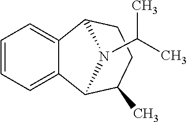 |
| 48 | 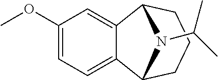 [SMILES: COc1ccc2c(c1)[C@@H]1CCC[C@H]2N1C(C)C] |
| 49 | 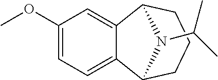 [SMILES: COc1ccc2c(c1)[C@H]1CCC[C@@H]2N1C(C)C] |

In some embodiments, the compounds of the present disclosure have a general structure of Formula (1-2):

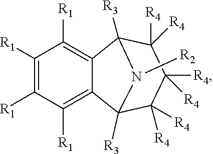

or a pharmaceutically acceptable salt thereof,

wherein R1 is independently H, optionally substituted C1 to C4 alkyl (including but not limited to fluoroalkyl, alkoxy, or amino alkyl), optionally substituted cycloalkyl (such as 3-8 membered cycloalkyl), halogen, or alkoxyl, and wherein any two adjacent R1 groups, together with the carbon to which they are attached, may form a 3 to 8-membered carbocycle or heterocycle which may be optionally substituted;

R2 is H, optionally substituted lower alkyl, cycloalkyl (such as 3-8 membered cycloalkyl), heterocycloalkyl, a multicyclic group, fluoroalkyl, or alkoxy; wherein R2 and any R4, together with the nitrogen and carbon to which they are attached, may form a 5 to 7-membered heterocycle which may be optionally substituted:

R3 is independently H, optionally substituted C1 to C4 alkyl or cycloalkyl (such as 3-8 membered cycloalkyl); wherein R3 and any adjacent R4, together with the carbons to which they are attached, may form a 5 to 7-membered carbocycle or heterocycle which may be optionally substituted; and

R4, can independently be H, optionally substituted C1 to C4 alkyl (including but not limited to fluoroalkyl, ether, amino alkyl, arylalkyl, heteroarylalkyl, or heterocycloalkyl), hydroxyl, halogen (such as F), or alkoxyl; wherein any two geminal R4 groups, together with the carbon to which they are attached, may form a 3 to 6-membered carbocycle or heterocycle which may be optionally substituted.

In some embodiments, two adjacent R1 groups form a 3 to 8-membered heterocycle, wherein the heterocycle comprises at least one oxygen, such as 2 oxygens.

In some embodiments, R2 is a lower alkyl group. In some embodiments, the alkyl group may be substituted with at least one functional group, such as a hydroxyl group, an alkoxyl group, an aryloxy group, an amino group, an aryl group, or a halogen. In some embodiments, there is a substituent (such as but not limited to a methyl group) at the alpha position of the alkyl group.

In some embodiments, R2 is a cyclic alkyl group.

In some embodiments, R2 and any R4, together with the nitrogen and carbon to which they are attached, may form a 5 to 8-membered non-aromatic heterocycle which may be optionally substituted.

In some embodiments, R3 and any R4, together with the carbons to which they are attached, may form a 5 to 8-membered non-aromatic carbocycle or heterocycle which may be optionally substituted.

In some embodiments, the two adjacent R4 groups, together with the carbon(s) to which they are attached, form a 5 to 8-membered aromatic carbocycle or heterocycle which may be optionally substituted. In some embodiments, the two adjacent R4 groups, together with the carbon(s) to which they are attached, form a 3 to 8-membered non-aromatic carbocycle or heterocycle which may be optionally substituted.

Non-limiting examples of compounds encompassed by Formula (1-2) include Compounds 1-38 and 40-49 or a pharmaceutically acceptable salt thereof.

In some embodiments, the compounds of the present disclosure have a general structure of Formula (I-3):

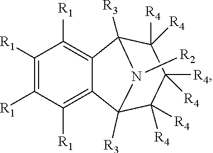

or a pharmaceutically acceptable salt thereof.

wherein R1 is independently H, optionally substituted C1 to C4 alkyl, optionally substituted cycloalkyl (such as 3-8 membered cycloalkyl), fluoroalkyl, ether, hydroxyl, halogen, or alkoxyl, and wherein any two adjacent R1 groups, together with the carbon to which they are attached, may form a 3 to 8-membered carbocycle or heterocycle which may be optionally substituted;

R2 is H, optionally substituted C1 to C7 alkyl, optionally substituted cycloalkyl (such as 3-8 membered cycloalkyl), heterocycloalkyl, a multicyclic group, fluoroalkyl, or ether;

R3 is independently H or optionally substituted C1 to C3 alkyl;

R4, can independently be H, optionally substituted C1 to C4 alkyl, fluoroalkyl, ether, hydroxyl, halogen (such as F), or alkoxyl.

In some embodiments, R2 is a C1 to C7 alkyl group. In some embodiments, the alkyl group may be substituted with at least one functional group, such as a hydroxyl group, an alkoxyl group, an aryloxy group, an amino group, an aryl group, or a halogen. In some embodiments, there is a substituent (such as a methyl group) at the alpha position of the alkyl group.

In some embodiments, R2 is a cyclic alkyl group.

In some embodiments, R4 is independently H, optionally substituted C1 to C4 alkyl, or fluoroalkyl.

Non-limiting examples of compounds encompassed by Formula (I-3) include Compounds 1-38 and 40-49 or a pharmaceutically acceptable salt thereof.

Generic Structure (II):

some embodiments, the compounds of the present disclosure have a general structure of Formula (II):

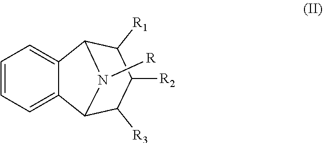

or a pharmaceutically acceptable salt thereof,

wherein R is hydrogen, an optionally substituted alkyl group, an optionally substituted cycloalkyl group (such as 3-8 membered cycloalkyl), an optionally substituted heterocyclic group, or an optionally substituted multicyclic group; and

R1, R2, or R3, independently, is hydrogen, an optionally substituted alkyl group, an optionally substituted cycloalkyl group (such as 3-8 membered cycloalkyl), a hydroxyl group, an alkoxy group, an aryloxy group, an amino group, or a halogen.

In some embodiments, at least one of R1, R2, or R3 is hydrogen. In some embodiments, R1, R2, and R3 are all hydrogens.

In some embodiments, R1, R2 or R3, independently, may be —OH, —OCH3 or a halogen.

In some embodiments, R, R1, R2, or R3 is an alkyl group. In some embodiments, the alkyl group may be substituted with at least one functional group, such as a hydroxyl group, an alkoxyl group, an aryloxy group, an amino group, an aryl group, or a halogen. In some embodiments, R, R1, R2 or R3 is a lower alkyl group. In some embodiments, R, R1, R2, or R3 is a cyclic alkyl group.

In some embodiments, R is an alkyl group. In some embodiments, the alkyl group is a lower alkyl group. In some embodiments, the alkyl group may be substituted with at least one functional group, such as a hydroxyl group, an alkoxyl group, an aryloxy group, an amino group, an aryl group, or a halogen. In some embodiments, there is a substituent (such as a methyl group) at the alpha position of the alkyl group.

In some embodiments, R is a cyclic alkyl group, such as 3-8 membered cycloalkyl.

In some embodiments, R is a heterocyclic group which may comprise O, N. or S.

In some embodiments, R is a multicyclic group which may be a bicyclic group. In some embodiments, the multicyclic group may include an unsaturated ring.

Non-limiting examples of compounds encompassed by Formula (11) include Compounds 1-38, 46 or 47 or a pharmaceutically acceptable salt thereof.

When R1, R2 and R3 are all hydrogens, non-limiting examples of compounds encompassed by Formula (11) include Compounds 1-26 or a pharmaceutically acceptable salt thereof:

TABLE 2
Non-limiting examples of compounds encompassed
by Formula (II)

| Compound No. | Structures |
|---|---|
| 1 | 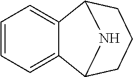 [SMILES: c1ccc2c(c1)C1CCCC2N1] |
| 2 | 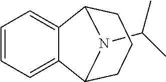 |
| 3 | 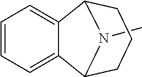 |
| 4 | 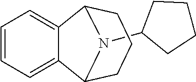 [SMILES: c1ccc2c(c1)C1CCCC2N1C1CCCC1] |
| 5 | 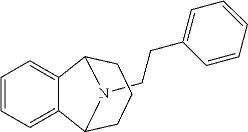 [SMILES: c1ccc(CCN2C3CCCC2c2ccccc23)cc1] |
| 6 | 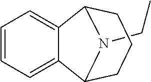 |
| 7 | 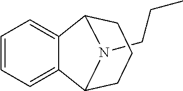 |
| 8 | 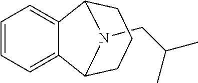 [SMILES: CC(C)CN1C2CCCC1c1ccccc12] |
| 9 | 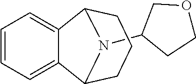 [SMILES: c1ccc2c(c1)C1CCCC2N1C1CCOC1] |
| 10 | 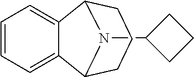 [SMILES: c1ccc2c(c1)C1CCCC2N1C1CCC1] |
| 11 | 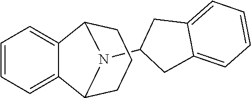 [SMILES: c1ccc2c(c1)CC(N1C3CCCC1c1ccccc13)C2] |
| 12 | 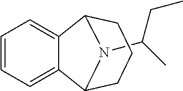 [SMILES: CCC(C)N1C2CCCC1c1ccccc12] |
| 13 | 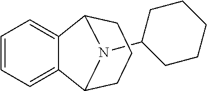 [SMILES: c1ccc2c(c1)C1CCCC2N1C1CCCCC1] |
| 14 | 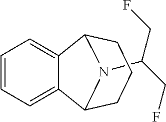 [SMILES: FCC(CF)N1C2CCCC1c1ccccc12] |
| 15 | 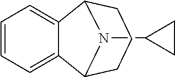 [SMILES: c1ccc2c(c1)C1CCCC2N1C1CC1] |
| 16 | 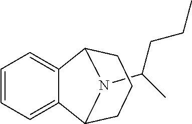 [SMILES: CCCC(C)N1C2CCCC1c1ccccc12] |
| 17 | 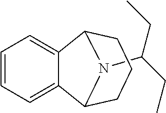 [SMILES: CCC(CC)N1C2CCCC1c1ccccc12] |
| 18 | 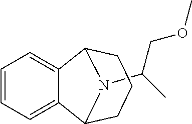 [SMILES: COCC(C)N1C2CCCC1c1ccccc12] |
| 19 | 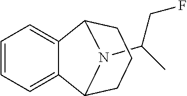 [SMILES: CC(CF)N1C2CCCC1c1ccccc12] |
| 20 | 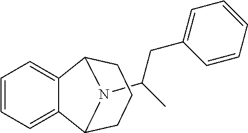 [SMILES: CC(Cc1ccccc1)N1C2CCCC1c1ccccc12] |
| 21 | 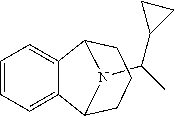 [SMILES: CC(C1CC1)N1C2CCCC1c1ccccc12] |
| 22 | 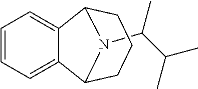 [SMILES: CC(C)C(C)N1C2CCCC1c1ccccc12] |
| 23 | 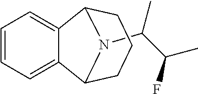 [SMILES: CC([C@@H](C)F)N1C2CCCC1c1ccccc12] |
| 24 | 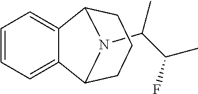 [SMILES: CC([C@H](C)F)N1C2CCCC1c1ccccc12] |
| 25 | 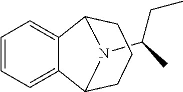 [SMILES: CC[C@@H](C)N1C2CCCC1c1ccccc12] |
| 26 | 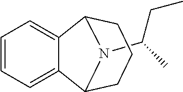 [SMILES: CC[C@H](C)N1C2CCCC1c1ccccc12] |

When at least one of R1, R2 and R3 is not hydrogen, non-limiting examples of compounds encompassed by Formula (11) include Compounds 27-38, 46 and 47, or a pharmaceutically acceptable salt thereof:

| Compound No. | Structures |
|---|---|
| 27 | 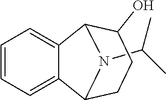 [SMILES: CC(C)N1C2CCC(O)C1c1ccccc12] |
| 28 | 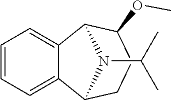 [SMILES: CO[C@H]1CC[C@H]2c3ccccc3[C@@H]1N2C(C)C] |
| 29 | 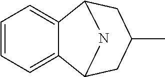 |
| 30 | 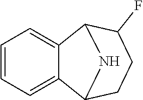 [SMILES: FC1CCC2NC1c1ccccc12] |
| 31 | 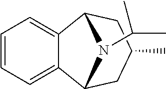 [SMILES: CC(C)N1[C@@H]2C[C@@H](C)C[C@H]1c1ccccc12] |
| 32 | 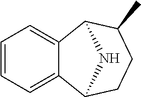 |
| 33 | 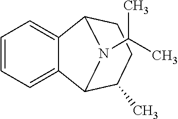 [SMILES: CC(C)N1C2CC[C@H](C)C1c1ccccc12] |
| 34 | 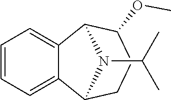 [SMILES: CO[C@@H]1CC[C@H]2c3ccccc3[C@@H]1N2C(C)C] |
| 35 | 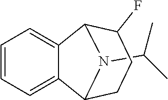 [SMILES: CC(C)N1C2CCC(F)C1c1ccccc12] |
| 36 | 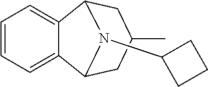 [SMILES: CC1CC2c3ccccc3C(C1)N2C1CCC1] |
| 37 | 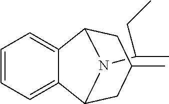 [SMILES: CCC(C)N1C2CC(C)CC1c1ccccc12] |
| 38 | 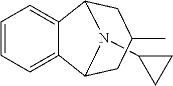 [SMILES: CC1CC2c3ccccc3C(C1)N2C1CC1] |
| 46 | 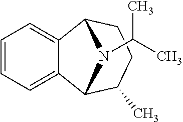 [SMILES: CC(C)N1[C@H]2CC[C@H](C)[C@@H]1c1ccccc12] |
| 47 | 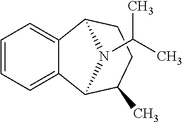 [SMILES: CC(C)N1[C@@H]2CC[C@@H](C)[C@H]1c1ccccc12] |

Generic Structure (III):

In some embodiments, the compounds of the present disclosure have a general structure of Formula (III):

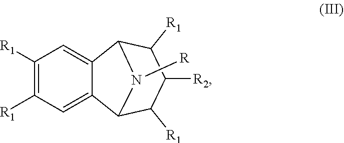

or a pharmaceutically acceptable salt thereof,

wherein R is hydrogen, an optionally substituted alkyl group, an optionally substituted cycloalkyl group (such as 3-8 membered cycloalkyl), an optionally substituted heterocyclic group, or an optionally substituted multicyclic group;

R1, R2, or R3, independently, is hydrogen, an optionally substituted alkyl group, an optionally substituted cycloalkyl group (such as 3-8 membered cycloalkyl), a hydroxyl group, an alkoxy group, an aryloxy group, an amino group, or a halogen; wherein any two adjacent R1, R2, or R3 groups, together with the carbons to which they are attached, may form a 5 to 8-membered carbocycle or heterocycle which may be optionally substituted; and

R4 or R5, independently, is H, optionally substituted lower alkyl (including but not limited to haloalkyl, alkoxy, amino alkyl, arylalkyl, or heteroarylalkyl), an optionally substituted cycloalkyl group (such as 3-8 membered cycloalkyl), halogen, hydroxyl, alkoxyl, ether, CN, amine, aryl, or heteroaryl; wherein R4 and R5 groups, together with the carbons to which they are attached, may form a 5 to 8-membered carbocycle or heterocycle which may be optionally substituted.

In some embodiments, at least one of R4 and R5 is a halogen. In some embodiments, both R4 and R5 are halogens. In some embodiments, both R4 and R5 are F. In some embodiments, at least one of R4 and R5 is an alkoxyl group, such as —OCH3. In some embodiments, R4 and R5 together with the carbon they are attached form

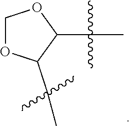 [SMILES: *C1OCOC1*]

Non-limiting examples of compounds encompassed by Formula (II) include Compounds 40-45, 48 and 49 or a pharmaceutically acceptable salt thereof:

TABLE 3
Non-limiting examples of compounds encompassed
by Formula (III)

| | |
|---|---|
| 40 | 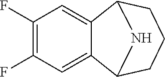 [SMILES: Fc1cc2c(cc1F)C1CCCC2N1] |
| 41 | 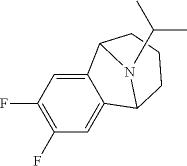 [SMILES: CC(C)N1C2CCCC1c1cc(F)c(F)cc12] |
| 42 | 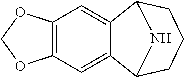 [SMILES: c1c2c(cc3c1C1CCCC3N1)OCO2] |
| 43 | 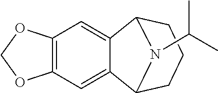 [SMILES: CC(C)N1C2CCCC1c1cc3c(cc12)OCO3] |
| 44 | 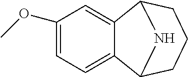 |
| 45 | 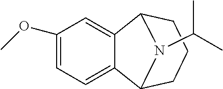 [SMILES: COc1ccc2c(c1)C1CCCC2N1C(C)C] |
| 48 | 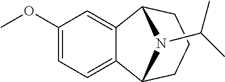 [SMILES: COc1ccc2c(c1)[C@@H]1CCC[C@H]2N1C(C)C] |
| 49 | 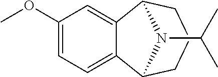 [SMILES: COc1ccc2c(c1)[C@H]1CCC[C@@H]2N1C(C)C] |

In some embodiments, the compound is Compound 39 having a structure of

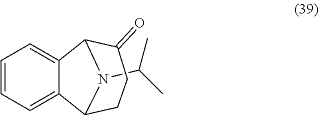 [SMILES: CC(C)N1C2CCC(=O)C1c1ccccc12]

or its pharmaceutically acceptable salt.

### II. Formulations

In some embodiments, compositions are administered to humans, human patients or subjects. For the purposes of the present disclosure, the phrase “active ingredient” generally refers to the conjugate as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%. e.g., between 0.5 and 50%, between 1-30%, between 5-80%, at least 80% (w/w) active ingredient.

The compounds of the present disclosure can be formulated using one or more excipients to: (1) increase stability; (2) permit the sustained or delayed release (e.g., from a depot formulation of the monomaleimide); (3) alter the biodistribution (e.g., target the monomaleimide compounds to specific tissues or cell types). (4) alter the release profile of the compounds in vivo. Non-limiting examples of the excipients include any and all solvents, dispersion media, diluents, or other liquid vehicles, dispersion or suspension aids, surface active agents, isotonic agents, thickening or emulsifying agents, and preservatives. Excipients of the present disclosure may also include, without limitation, lipidoids, liposomes, lipid nanoparticles, polymers, lipoplexes, core-shell nanoparticles, peptides, proteins, hyaluronidase, nanoparticle mimics and combinations thereof. Accordingly, the formulations of the disclosure may include one or more excipients, each in an amount that together increases the stability of the monomaleimide compounds.

### Excipients

Pharmaceutical formulations may comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy. 21st Edition, A. R. Gennaro (Lippincott. Williams & Wilkins, Baltimore. Md., 2006; incorporated herein by reference in its entirety) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia. and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEEN®6], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ®45], polyoxyethylene hydrogenated castor oil, polyethoxylated castor oil, polyoxymethylene stearate, and Kolliphor® (SOLUTOL®)), sucrose fatty acid esters, polyethylene glycol fatty acid esters (e.g. CREMOPHOR®), polyoxyethylene ethers, (e.g. polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC®F 68, POLOXAMER®188, cetrimonium bromide, cetylpyridinium chloride, benzalkonium chloride, docusate sodium, and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan); alginates; polyethylene oxide; polyethylene glycol; inorganic calcium salts, silicic acid, polymethacrylates; waxes, water; alcohol; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN®II, NEOLONE™, KATHON™, and/or EUXYL®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macadamia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition, according to the judgment of the formulator.

### III. Methods of Using the Compounds

In various embodiments, methods of using the compounds are provided, wherein the method comprises administering a therapeutically effective amount of the compounds or pharmaceutically acceptable salts thereof, as described herein, to a subject. The subject may have a CNS disorder, may be suspected of having a CNS disorder, or may have a predisposition to a CNS disorder. The compounds or pharmaceutically acceptable salts thereof are administered to the subject as a treatment for a CNS disorder and maintenance in all patients (including both the acute phase of the CNS disorder and as a maintenance therapeutic for the CNS disorder).

CNS disorders affect a wide range of the population with differing severity. Neurological and psychiatric disorders include but not limited to depression (such as treatment-resistant depression (TRD), major depressive disorder (MDD), bipolar depression, unipolar depression, or depression associated with another disease or disorder), anxiety, cognitive impairment, schizophrenia, bipolar disorder, obsessive compulsive disorder (OCD), panic disorder, posttraumatic stress disorder (PTSD), addiction, social disorder, attention deficit hyperactivity disorder (ADHD), autism, neuropsychiatric symptoms such as apathy, depression, anxiety, psychosis, aggression, agitation, poor impulse control, and sleep disruptions in neurological disorders such as Alzheimer's and Parkinson's diseases. These disorders and symptoms affect a person's thoughts, mood, behavior and social interactions and can significantly impair daily functioning.

In some embodiments, the compounds of the present disclosure are used to treat one or more symptoms of CNS disorders, such as but not limited to depression (e.g., major depressive disorder or dysthymia); bipolar disorder, seasonal affective disorder; cognitive deficit; sleep related disorder (e.g., sleep apnea, insomnia, narcolepsy, cataplexy) including those sleep disorders which are produced by psychiatric conditions: chronic fatigue syndrome; anxieties (e.g., general anxiety disorder, social anxiety disorder, panic disorder); obsessive compulsive disorder; post-menopausal vasomotor symptoms (e.g., hot flashes, night sweats): neurodegenerative disease (e.g., Parkinson's disease, Alzheimer's disease and amyotrophic lateral sclerosis); manic disorder; dysthymic disorder; obesity; acute suicidality or suicide ideation; suicidal behavior disorder; senile dementia; Alzheimer's type dementia; cognition, memory loss; amnesia/amnestic syndrome; disturbances of consciousness; coma; lowering of attention; speech disorder; Lennox syndrome; hyperkinetic syndrome; neuropathic pain, including post herpetic (or post-shingles) neuralgia, reflex sympathetic dystrophy/causalgia or nerve trauma, phantom limb pain, carpal tunnel syndrome, and peripheral neuropathy (such as diabetic neuropathy or neuropathy arising from chronic alcohol use); migraine or migraine headache; sexual dysfunction, in men or women, including sexual dysfunction caused by psychological and/or physiological factors, erectile dysfunction, premature ejaculation, vaginal dryness, lack of sexual excitement, inability to obtain orgasm, and psycho-sexual dysfunction, including inhibited sexual desire, inhibited sexual excitement, inhibited female orgasm, inhibited male orgasm, functional dyspareunia, functional vaginismus, and atypical psychosexual dysfunction; rapid eye movement (REM) during both sleep and daytime equivalent; cataplexy (sudden involuntary transient bouts of muscle weakness or paralysis while awake); nighttime sleep disturbance/sleep fragmentation associated with narcolepsy or other conditions; and sleep paralysis associated with narcolepsy or other conditions: hypnagogic and hypnapompic hallucinations associated with narcolepsy or other conditions: and excessive daytime sleepiness associated with narcolepsy, sleep apnea or shift work disorder and other medical conditions such as cancer, chronic fatigue syndrome and fibromyalgia.

### Depression

Depression (or Major depressive disorder (MDD)), is a CNS disorder characterized by at least 2 weeks of low mood across most situations, often accompanied by low self-esteem, loss of interest in normally enjoyable activities, low energy, and pain without a clear cause. Depression may be unipolar or bipolar. For patients who have been diagnosed with bipolar disorder and have an episode of mania or markedly elevated mood, the depression episode is called bipolar depression. Depression without mania is sometimes referred to as unipolar because the mood remains at one emotional state. Symptoms of depression include anhedonia, depressed mood (sadness), poor concentration, hopelessness, poor self-esteem, insomnia, fatigue, appetite disturbances, generalized symptoms of pain, excessive guilt and thoughts of suicide.

Bipolar disorder is a severe, recurrent, lifelong psychiatric illness that affects a lot of adult Americans and imposes significant economic burden to patients, families, and society. Although the bipolar mania phase can be reasonably well controlled with existing medications (e.g. lithium), the treatment of bipolar depression (BPD) relies on repurposing older classes of antipsychotic and anticonvulsant drugs. These older drugs have limited efficacy in treating the symptoms of BPD and many are concomitant with adverse side effects and reduced tolerability. Consequently, nonadherence to medication is common and BPD is associated with high morbidity, substance abuse, and a high rate of patient suicide. Thus, there remains a significant medical need for the focused discovery and development of new, safe, and effective drugs for the treatment of BPD.

Medications that work well in the manic phase of bipolar disorder have been repurposed from other mental health disorders (e.g. schizophrenia, major depressive disorder), and frequently fail to treat the depressive phase of the illness. Although the broad consensus is that antispychotics (e.g. quetiapine, olanzapine, lurasidone) and/or antiepileptics (e.g. valproate, lamotrignine, carbamazapine) can help stabilize moods, many patients with bipolar depression do not respond adequately to these medications, despite adequate clinical trials. Older medications such as lithium similarly have variable and modest efficacy in the treatment of depression and relapse prevention.

Another limitation of existing mood stabilizing drugs is that they are associated with a considerable lag of onset. Only a fraction of patients meet response criteria by the end of the first week of treatment, and continued use is associated with many undesirable side effects. Slow therapeutic onset contributes to the life disruptions experienced by individuals, and the delay in treating suicidal behavior is an issue of particular concem for this already vulnerable population.

The present disclosure provides methods of treating depression (such as but not limited to bipolar depression, unipolar depression, major depressive disorder, or treatment-resistant depression) or maintenance therapy of depression (such as but not limited to bipolar depression, unipolar depression, major depressive disorder, or treatment-resistant depression), wherein the method comprises administering a therapeutically effective amount of the compounds of the present disclosure or pharmaceutically acceptable salts thereof

### Psychosis

Psychosis is a group of disorders including schizophrenia (paranoid, disorganized, catatonic or undifferentiated), schizophreniform disorder, schizoaffective disorder, delusional disorder, brief psychotic disorder, shared psychotic disorder, psychotic disorder due to a general medical condition and substance-induced or drug-induced (e.g., phencyclidine, ketamine and other dissociative anesthetics, amphetamine and other psychostimulants and cocaine) psychosis, psychotic disorders, psychosis associated with affective disorders, brief reactive psychosis, schizoaffective psychosis, “schizophrenia-spectrum” disorders such as schizoid or schizotypal personality disorders, or illness associated with psychosis (such as major depression, manic depressive (bipolar) disorder, Alzheimer's disease and post-traumatic stress syndrome), including both positive, negative, and cognitive symptoms of schizophrenia and other psychoses

Schizophrenia is a psychopathic disorder marked by characteristics such as psychotic symptoms, phasic progression and development, and/or deterioration in social behavior and professional capability. It usually appears for the first time in early adulthood. Characteristic psychotic symptoms are disorders of thought content (e.g., multiple, fragmentary, incoherent, implausible or simply delusional contents, or ideas of persecution) and of mentality (e.g., loss of association, flight of imagination, incoherence up to incomprehensibility), as well as disorders of perceptibility (e.g., hallucinations), emotions (e.g., superficial or inadequate emotions), self-perceptions, intentions, impulses, and/or inter-human relationships, and psychomotoric disorders (e.g., catatonia).

Schizophrenia is classified into subgroups: the paranoid type, the disorganized type, the catatonic type, and the undifferentiated type. The paranoid subgroup is characterized by delusions and hallucinations and absence of thought disorder, disorganized behavior, and affective flattening. Thought disorder and flat affect are present together in the disorganized type, also named “hebephrenic schizophrenia” Prominent psychomotor disturbances are evident in the catatonic type, wherein symptoms may include catatonic stupor and waxy flexibility. In the undifferentiated type, psychotic symptoms are present but the criteria for paranoid, disorganized, or catatonic types have not been met.

The symptoms of schizophrenia include three broad categories: positive, negative and cognitive symptoms. Positive symptoms are those which represent an “excess” of normal experiences, such as hallucinations and delusions. Negative symptoms are those where the patient suffers from a lack of normal experiences, such as anhedonia and lack of social interaction. The cognitive symptoms relate to cognitive impairment in schizophrenics, such as lack of sustained attention and deficits in decision making.

The present disclosure provides methods of treating psychosis (such as schizophrenia) or maintenance therapy of psychosis (such as schizophrenia), wherein the method comprises administering a therapeutically effective amount of the compounds of the present disclosure or pharmaceutically acceptable salts thereof.

### Cognitive Disorders & Cognitive Impairment

Cognitive disorders include dementia (semantic dementia, frontotemporal dementia, dementia with depressive features, persisting, subcortical dementia, dementia with Lewy Bodies, Parkinsonism-ALS Dementia Complex, and dementia associated with Alzheimer's disease, ischemia, multi-infarct dementia, trauma, vascular problems, stroke, HIV disease, Parkinson's disease, Huntington's disease, Down syndrome, Pick's disease, Creutzfeldt-Jacob disease, perinatal hypoxia, or substance abuse), delirium, amnestic disorders or age related cognitive decline.

Cognitive impairment includes a decline in cognitive functions or cognitive domains. e.g., working memory, attention and vigilance, verbal learning and memory, visual learning and memory, reasoning and problem solving (e.g., executive function, speed of processing and/or social cognition). In particular, cognitive impairment may indicate deficits in attention, disorganized thinking, slow thinking, difficulty in understanding, poor concentration, impairment of problem solving, poor memory, difficulties in expressing thoughts, and/or difficulties in integrating thoughts, feelings and behavior, or difficulties in extinction of irrelevant thoughts.

The present disclosure provides methods of treating cognitive disorders and/or cognitive impairment or maintenance therapy of cognitive disorders and/or cognitive impairment, wherein the method comprises administering a therapeutically effective amount of the compounds of the present disclosure or pharmaceutically acceptable salts thereof.

### Anxiety

Anxiety disorders are disorders characterized by fear, worry, and uneasiness, usually generalized and unfocused as an overreaction to a situation. Anxiety disorders differ in the situations or types of objects that induce fear, anxiety, or avoidance behavior, and the associated cognitive ideation. Anxiety differs from fear in that anxiety is an emotional response to a perceived future threat while fear is associated with a perceived or real immediate threat. They also differ in the content of the associated thoughts or beliefs.

Anxiety disorders including acute stress disorder, agoraphobia, generalized anxiety disorder, obsessive-compulsive disorder, panic attack, panic disorder, post-traumatic stress disorder, separation anxiety disorder, social phobia, specific phobia, substance-induced anxiety disorder and anxiety due to a general medical condition.

The present disclosure provides methods of treating anxiety or maintenance therapy of anxiety, wherein the method comprises administering a therapeutically effective amount of the compounds of the present disclosure or pharmaceutically acceptable salts thereof.

### Administration

The compounds of the present disclosure may be administered by any route which results in a therapeutically effective outcome. These include, but are not limited to enteral, gastroenteral, epidural, oral, transdermal, epidural (peridural), intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection, (into the base of the penis), intravaginal administration, intrauterine, extra-amniotic administration, transdermal (diffusion through the intact skin for systemic distribution), transmucosal (diffusion through a mucous membrane), insufflation (snorting), sublingual, sublabial, enema, eye drops (onto the conjunctiva), or in ear drops. In specific embodiments, compositions may be administered in a way which allows them to cross the blood-brain barrier, vascular barrier, or other epithelial barrier.

In some embodiments, the compounds are administered orally. The oral formulations contain an effective amount of compounds in a pharmaceutical carrier appropriate for administration to an individual in need thereof.

### Dosing

The present disclosure provides methods comprising administering compounds as described herein to a subject in need thereof. Compounds as described herein may be administered to a subject using any amount and any route of administration effective for preventing or treating or imaging a disease, disorder, and/or condition (e.g., a disease, disorder, and/or condition relating to working memory deficits). The exact amount required will vanv from subject to subject, depending on the species, age, and general condition of the subject, the severity of the disease, the particular composition, its mode of administration, its mode of activity, and the like.

Compositions in accordance with the disclosure are typically formulated in dosage unit form for ease of administration and uniformity of dosage. It will be understood, however, that the total daily usage of the compositions of the present disclosure may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, compositions in accordance with the present disclosure may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, from about 25 mg/kg to about 50 mg/kg, from about 50 mg/kg to about 100 mg/kg, from about 100 mg/kg to about 125 mg/kg, from about 125 mg/kg to about 150 mg/kg, from about 150 mg/to about 175 mg/kg, from about 175 mg/kg to about 200 mg/kg, from about 200 mg/kg to about 250 mg/kg of subject body weight per day, one or more times a day, to obtain the desired therapeutic, diagnostic, prophylactic, or imaging effect. The desired dosage may be delivered three times a day, two times a day, once a day, every other day, every third day, every week, every two weeks, every three weeks, or every four weeks. In some embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used.

As used herein, a “split dose” is the division of single unit dose or total daily dose into two or more doses. e.g. two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in 24 hr period. It may be administered as a single unit dose.

Dosage Forms

A pharmaceutical composition described herein can be formulated into a dosage form described herein, such as a topical, intranasal, intratracheal, or injectable (e.g., intravenous, intraocular, intravitreal, intramuscular, intracardiac, intraperitoneal, and subcutaneous).

### Coatings or Shells

Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

### Liquid Dosage Forms

Liquid dosage forms for parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art including, but not limited to, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. In certain embodiments for parenteral administration, compositions may be mixed with solubilizing agents such as CREMOPHOR®, alcohols, oils, modified oils, glycols, polysorbates, cyclodextrins, polymers, and/or combinations thereof.

### Injectable

Injectable preparations, for example, sterile injectable aqueous or oleaginous suspensions may be formulated according to the known art and may include suitable dispersing agents, wetting agents, and/or suspending agents. Sterile injectable preparations may be sterile injectable solutions, suspensions, and/or emulsions in nontoxic parenterally acceptable diluents and/or solvents, for example, a solution in 1,3-butanediol. Among the acceptable vehicles and solvents that may be employed include, but are not limited to, water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it may be desirable to slow the absorption of the active ingredient from subcutaneous or intramuscular injection. This may be accomplished by the use of a liquid suspension of crystalline or amorphous material with poor water solubility. The rate of absorption of the compounds then depends upon its rate of dissolution which, in turn, may depend upon crystal size and crystalline form. Alternatively, delayed absorption of a parenterally administered compound may be accomplished by dissolving or suspending the compound in an oil vehicle. Injectable depot forms are made by forming microencapsule matrices of the compounds in biodegradable polymers such as polylactide-polyglycolide. Depending upon the ratio of compounds to polymer and the nature of the particular polymer employed, the rate of compound release can be controlled. Examples of other biodegradable polymers include, but are not limited to, poly(orthoesters) and poly(anhydrides). Depot injectable formulations may be prepared by entrapping the compounds in liposomes or microemulsions which are compatible with body tissues.

### Pulmonary

Formulations described herein as being useful for pulmonary delivery may also be used for intranasal delivery of a pharmaceutical composition. Another formulation suitable for intranasal administration may be a coarse powder comprising the active ingredient and having an average particle from about 0.2 μm to 500 μm. Such a formulation may be administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, contain about 0.1% to 20% (w/w) active ingredient, where the balance may comprise an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

General considerations in the formulation and/or manufacture of pharmaceutical agents may be found, for example, in Remington: The Science and Practice of Pharmacy 21st ed., Lippincott Williams & Wilkins, 2005 (incorporated herein by reference in its entirety).

### Combo Therapies

In some embodiments, the present disclosure provides a method of treating a neurological and/or psychiatric disease or disorder described herein, comprising administering a compound of the present disclosure in combination with one or more additional active agents or therapies. Suitable pharmaceutical agents that may be used in combination with the compounds of the present disclosure include antidepressants, anti-psychotics, anti-Parkinson's drugs, anti-Alzheimer's drugs, anti-ischemics. CNS depressants, anti-cholinergics, nootropics, epilepsy medication, attention (e.g., ADD/ADHD) medications, sleep-promoting medications, wakefulness-promoting medications, pain medications, or anxiolytics.

The compounds of the present disclosure and the additional active agent(s) may be administered simultaneously, sequentially, or at any order. The compounds of the present disclosure and the additional active agent(s) may be administered at different dosages, with different dosing frequencies, or via different routes, whichever is suitable.

### IV. Kits and Devices

The disclosure provides a variety of kits and devices for conveniently and/or effectively carrying out methods of the present disclosure. Typically, kits will comprise sufficient amounts and/or numbers of components to allow a user to perform multiple treatments of a subject(s) and/or to perform multiple experiments.

In one embodiment, the present disclosure provides kits for treating CNS disorders, comprising a compound of the present disclosure or a combination of compounds of the present disclosure, optionally in combination with any other active agents.

The kit may further comprise packaging and instructions and/or a delivery agent to form a formulation composition. The delivery agent may comprise a saline, a buffered solution, or any delivery agent disclosed herein. The amount of each component may be varied to enable consistent, reproducible higher concentration saline or simple buffer formulations. The components may also be varied in order to increase the stability of the compound(s) in the buffer solution over a period of time and/or under a variety of conditions.

The present disclosure provides for devices which may incorporate compound(s) of the present disclosure. These devices contain in a stable formulation available to be immediately delivered to a subject in need thereof, such as a human patient. In some embodiments, the subject has BPD.

Non-limiting examples of the devices include a pump, a catheter, a needle, a transdermal patch, a pressurized olfactory delivery device, iontophoresis devices, multi-layered microfluidic devices. The devices may be employed to deliver compound(s) of the present disclosure according to single, multi- or split-dosing regiments. The devices may be employed to deliver compound(s) of the present disclosure across biological tissue, intradermal, subcutaneously, or intramuscularly.

### V. Definitions

The abbreviations used herein have their conventional meaning within the scientific arts. The chemical elements are identified in accordance with the Periodic Table of the Elements, CAS version, Handbook of Chemistry and Physics, 75th Ed. Additionally, general principles of organic chemistry are described in M. Loudon, Organic Chemistry, 5th Ed., Roberts and Company, Greenwood Village, Colo.: 2009; and M. B. Smith, March's Advanced Organic Chemistry: Reactions, Mechanisms and Structure, 7th Ed., John Wiley & Sons, Hoboken: 2013, the entire contents of which are hereby incorporated by reference.

The term “compound”, as used herein, is meant to include all stereoisomers, geometric isomers, tautomers, and isotopes of the structures depicted.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Compounds of the present disclosure also include tautomeric forms. Tautomeric forms result from the swapping of a single bond with an adjacent double bond and the concomitant migration of a proton. Tautomeric forms include prototropic tautomers which are isomeric protonation states having the same empirical formula and total charge. Examples prototropic tautomers include ketone—enol pairs, amide—imidic acid pairs, lactam—lactim pairs, amide-imidic acid pairs, enamine—imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

Where substituent groups are specified by their conventional chemical formulae, written from left to right, they equally encompass the chemically identical substituents which would result from writing the structure from right to left, e.g., —CH₂O— is intended to also recite —OCH₂—; —NHS(O)₂— is also intended to represent —S(O)₂HN—; etc.

The term “alkyl,” by itself or as part of another substituent, means, unless otherwise stated, a straight or branched chain, or cyclic hydrocarbon radical (also called cycloalkyl or cyclic alkyl group), or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups, which are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present disclosure. A “lower alkyl” or “lower alkylene” is a shorter chain alkyl or alkylene group, generally having eight or fewer carbon atoms.

The terms “alkoxy,” (or “alkoxyl”) “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N, S and Si may be placed at any interior position of the heteroalkyl group or at the position at which the alkyl group is attached to the remainder of the molecule. Examples include, but are not limited to, —CH₂—CH₂O—CH, —CH₂—CH₂—NH—CH₃, —CH₂—CH₂—N(CH₃)—CH₃, —CH₂—S—CH₂—CH₃, —CH₂—CH₂. —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

In general, an “acyl substituent” is also selected from the group set forth above. As used herein, the term “acyl substituent” refers to groups attached to, and fulfilling the valence of a carbonyl carbon that is either directly or indirectly attached to the polycyclic nucleus of the compounds of the present disclosure.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C1-C4)alkyl” is mean to include, but not be limited to, trifluoromethyl, 2,2,2-trifluoroethyl. 4-chlorobutyl, 3-bromopropyl, and the like.

The term “aryl” means, unless otherwise stated, a polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl. 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 34(1-naphthyloxy)propyl, and the like).

The terms “carbocycle” and “heterocycle” refers to non-aromatic (such as “cycloalkyl” and “heterocycloalkyl” as defined herein) or aromatic (such as “aryl” and “heteroaryl” as defined herein) rings. The “carbocycle” and “heterocycle” groups may be saturated or non-saturated.

Each of the above terms (e.g., “alkyl,” “heteroalkyl,” “aryl,” “heteroaryl,” “carbocycle,” and “heterocycle”) include both substituted and unsubstituted forms of the indicated radical. Preferred substituents for each type of radical are provided below.

Substituents for the alkyl, and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generally referred to as “alkyl substituents” and “heteroalkyl substituents,” respectively, and they can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″. —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the disclosure includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, the aryl substituents and heteroaryl substituents are generally referred to as “aryl substituents” and “heteroaryl substituents,” respectively and are varied and selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, (C₁-C₅)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the disclosure includes more than one R group, for example, each of the R groups is independently selected as are each R′, R″, R′″ and R″″ groups when more than one of these groups is present.

Two of the aryl substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

The term “alkyl amide” refers to carboxylic acid amides that are functionalized on the amide nitrogen by one or more alkyl groups as defined herein.

The term “alkyl amine” refers to amines in which the nitrogen atom is functionalized with one or more alkyl groups as defined herein.

As used herein, the term “heteroatom” includes oxygen (O), nitrogen (N), sulfur (S) and silicon (Si).

The symbol “R” is a general abbreviation that represents a substituent group that is selected from substituted or unsubstituted alkyl, substituted or unsubstituted heteroalkyl, substituted or unsubstituted aryl, substituted or unsubstituted heteroaryl, and substituted or unsubstituted heterocyclyl groups.

The term “pharmaceutically acceptable salts” includes salts of the active compounds which are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science. 1977, 66, 1-19). Certain specific compounds of the present disclosure contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

The terms “subject” or “patient”, as used herein, refer to any organism to which the particles may be administered, e.g., for experimental, therapeutic, diagnostic, and/or prophylactic purposes. Typical subjects include animals (e.g., mammals such as mice, rats, rabbits, guinea pigs, cattle, pigs, sheep, horses, dogs, cats, hamsters, lamas, non-human primates, and humans).

The terms “treating” or “preventing”, as used herein, can include preventing a disease, disorder or condition from occurring in an animal that may be predisposed to the disease, disorder and/or condition but has not yet been diagnosed as having the disease, disorder or condition: inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

The terms “managing” or “maintaining”, as used herein, can refer to reducing the symptom(s) of a disease, reducing the severity of symptom(s) of the disease, or preventing the symptom(s) of the disease from getting worse.

The term “therapeutic effect” is art-recognized and refers to a local or systemic effect in animals, particularly mammals, and more particularly humans caused by a pharmacologically active substance. The term thus means any substance intended for use in the diagnosis, cure, mitigation, treatment or prevention of disease, disorder or condition in the enhancement of desirable physical or mental development and conditions in an animal, e.g., a human.

The term “modulation” is art-recognized and refers to up regulation (i.e., activation or stimulation), down regulation (i.e., inhibition or suppression) of a response, or the two in combination or apart. The modulation is generally compared to a baseline or reference that can be internal or external to the treated entity.

“Parenteral administration”, as used herein, means administration by any method other than through the digestive tract (enteral) or non-invasive topical routes. For example, parenteral administration may include administration to a patient intravenously, intradermally, intraperitoneally, intrapleurally, intratracheally, intraossiously, intracerebrally, intrathecally, intramuscularly, subcutaneously, subjunctivally, by injection, and by infusion.

“Topical administration”, as used herein, means the non-invasive administration to the skin, orifices, or mucosa. Topical administration can be delivered locally, i.e., the therapeutic can provide a local effect in the region of delivery without systemic exposure or with minimal systemic exposure. Some topical formulations can provide a systemic effect, e.g., via adsorption into the blood stream of the individual. Topical administration can include, but is not limited to, cutaneous and transdermal administration, buccal administration, intranasal administration, intravaginal administration, intravesical administration, ophthalmic administration, and rectal administration.

“Enteral administration”, as used herein, means administration via absorption through the gastrointestinal tract. Enteral administration can include oral and sublingual administration, gastric administration, or rectal administration.

“Pulmonary administration”, as used herein, means administration into the lungs by inhalation or endotracheal administration. As used herein, the term “inhalation” refers to intake of air to the alveoli. The intake of air can occur through the mouth or nose.

The terms “sufficient” and “effective”, as used interchangeably herein, refer to an amount (e.g., mass, volume, dosage, concentration, and/or time period) needed to achieve one or more desired result(s). A “therapeutically effective amount” is at least the minimum concentration required to affect a measurable improvement or prevention of at least one symptom or a particular condition or disorder, to affect a measurable enhancement of life expectancy, or to generally improve patient quality of life. The therapeutically effective amount is thus dependent upon the specific biologically active molecule and the specific condition or disorder to be treated. Therapeutically effective amounts of many active agents, such as antibodies, are known in the art. The therapeutically effective amounts of compounds and compositions described herein, e.g., for treating specific disorders may be determined by techniques that are well within the craft of a skilled artisan, such as a physician.

The terms “bioactive agent” and “active agent”, as used interchangeably herein, include, without limitation, physiologically or pharmacologically active substances that act locally or systemically in the body. A bioactive agent is a substance used for the treatment (e.g., therapeutic agent), prevention (e.g., prophylactic agent), diagnosis (e.g., diagnostic agent), cure or mitigation of disease or illness, a substance which affects the structure or function of the body, or pro-drugs, which become biologically active or more active after they have been placed in a predetermined physiological environment.

The term “pharmaceutically acceptable”, as used herein, refers to compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the U.S. Food and Drug Administration. A “pharmaceutically acceptable carrier”, as used herein, refers to all components of a pharmaceutical formulation that facilitate the delivery of the composition in vivo. Pharmaceutically acceptable carriers include, but are not limited to, diluents, preservatives, binders, lubricants, disintegrators, swelling agents, fillers, stabilizers, and combinations thereof.

The term “pharmaceutically acceptable salt(s)” refers to salts of acidic or basic groups that may be present in compounds used in the present compositions. Compounds included in the present compositions that are basic in nature are capable of forming a variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfate, citrate, malate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions, that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

If the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

A pharmaceutically acceptable salt can be derived from an acid selected from 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid, aspartic acid, benzenesulfonic acid, benzoic acid, camphoric acid, camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid, gluconic acid, glucuronic acid, glutamic acid, glutaric acid, glycerophosphoric acid, glycolic acid, hippuric acid, hydrobromic acid, hydrochloric acid, isethionic, isobutyric acid, lactic acid, lactobionic acid, lauric acid, maleic acid, malic acid, malonic acid, mandelic acid, methanesulfonic acid, mucic, naphthalene-1,5-disulfonic acid, naphthalene-2-sulfonic acid, nicotinic acid, nitric acid, oleic acid, oxalic acid, palmitic acid, pamoic acid, pantothenic, phosphoric acid, proprionic acid, pyroglutamic acid, salicylic acid, sebacic acid, stearic acid, succinic acid, sulfuric acid, tartaric acid, thiocyanic acid, toluenesulfonic acid, trifluoroacetic, and undecylenic acid.

The term “protective group”, as used herein, refers to a functional group that can be added to and/or substituted for another desired functional group to protect the desired functional group from certain reaction conditions and selectively removed and/or replaced to deprotect or expose the desired functional group. Protective groups are known to the skilled artisan. Suitable protective groups may include those described in Greene and Wuts, Protective Groups in Organic Synthesis, (1991). Acid sensitive protective groups include dimethoxytrityl (DMT), tert-butylcarbamate (tBoc) and trifluoroacetyl (tFA). Base sensitive protective groups include 9-fluorenylmethoxycarbonyl (Fmoc), isobutyrl (iBu), benzoyl (Bz) and phenoxyacetyl (pac). Other protective groups include acetamidomethyl, acetyl, tert-amyloxycarbonyl, benzyl, benzyloxycarbonyl, 2-(4-biphenylyl)-2-propy!oxycarbonyl, 2-bromobenzyloxycarbonyl, tert-butyl7 tert-butyloxycarbonyl, 1-carbobenzoxamido-2,2,2-trifluoroethyl, 2,6-dichlorobenzyl, 2-(3,5-dimethoxyphenyl)-2-propyloxycarbonyl, 2,4-dinitrophenyl, dithiasuccinyl, formyl, 4-methoxybenzenesulfonyl, 4-methoxybenzyl, 4-methylbenzyl, o-nitrophenylsulfenyl, 2-phenyl-2-propyloxycarbonyl, α-2,4,5-tetramethylbenzyloxycarbonyl, p-toluenesulfonyl, xanthenyl, benzyl ester, N-hydroxysuccinimide ester, p-nitrobenzyl ester, p-nitrophenyl ester, phenyl ester, p-nitrocarbonate, p-nitrobenzylcarbonate, trimethylsilyl and pentachlorophenyl ester.

The term “bioavailable” is art-recognized and refers to a form of the subject disclosure that allows for it, or a portion of the amount administered, to be absorbed by, incorporated to, or otherwise physiologically available to a subject or patient to whom it is administered.

As used herein, the behavioral effects identified will have the following definitions: AD: antidepressant means a behavior or behavioral pattern manifesting as or presenting evidence of or supportive of a conclusion of the reduction, alleviation or prevention of depression; AX: anxiolytic means a behavior or behavioral pattern manifesting as or presenting evidence of or supportive of a conclusion of the reduction, alleviation or prevention of anxiety; SD: sedative hypnotic means a behavior or behavioral pattern manifesting as or presenting evidence of or supportive of a conclusion of the promotion of calm or induction of sleep; AP: antipsychotic means a behavior or behavioral pattern manifesting as or presenting evidence of or supportive of a conclusion of the reduction, alleviation or prevention of one or more psychoses: MS: mood stabilizer means a behavior or behavioral pattern manifesting as or presenting evidence of or supportive of a conclusion of the stabilization of mood or establishment of a sense of well-being; CE: cognitive enhancer means a behavior or behavioral pattern manifesting as or presenting evidence of or supportive of a conclusion of an increase in the mental action or process of acquiring knowledge and understanding through experience (including the senses); AG: analgesic means a behavior or behavioral pattern manifesting as or presenting evidence of or supportive of a conclusion of the relief of pain: XG: anxiogenic (or panicogenic) means a behavior or behavioral pattern manifesting as or presenting evidence of or supportive of a conclusion of the increase, elevation or creation of anxiety; HA: hallucinogenic means a behavior or behavioral pattern manifesting as or presenting evidence of or supportive of a conclusion of the increase, elevation or creation of hallucinations (i.e., an experience involving the apparent perception of something not present); SE: side effect means a behavior or behavioral pattern manifesting as or presenting evidence of or supportive of a conclusion of the increase, elevation or creation of a secondary, typically undesirable effect of a drug or medical treatment; UN: uncharacterized CNS activity means a behavior or behavioral pattern manifesting as or presenting insufficient evidence to support a conclusion or correlation of any CNS effect of a drug or treatment.

The present disclosure is further illustrated by the following non-limiting examples.

### EXAMPLES

### Example 1. Synthesis of the Compounds

The compounds of the disclosure may be prepared using any convenient methodology known to a person of the art. Non-limiting synthetic methods for the compounds of the present disclosure are provided below.

Compounds 1 and 2

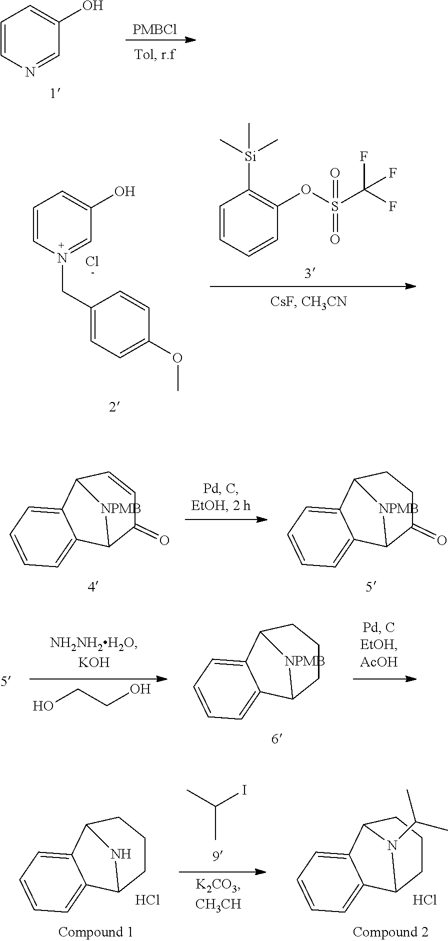

### Step 1

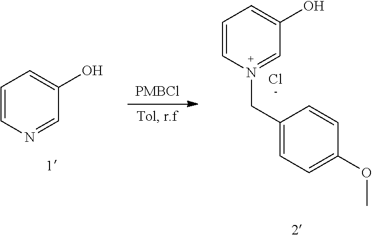

### 3-Hydroxy-1-[(4-methoxyphenyl)methyl]pyridin-1-ium chloride (2′)

Pyridin-3-ol (50 g, 0.52 mol) was dissolved in toluene (1 L). 1-(Chloromethyl)-4-methoxybenzene (82.3 g, 0.52 mol) was added, and the reaction mixture was stirred at 120° C. overnight. The solvent was evaporated, and the residue was washed with PE and filtered. The filter was dried in vacuo to give 2′ (130 g, 98%) as a white solid. LCMS. BONB-00122-082(ESI) m/z=215.9 (M+H)⁺, t=2.095 min (215 nm).

### Step 2

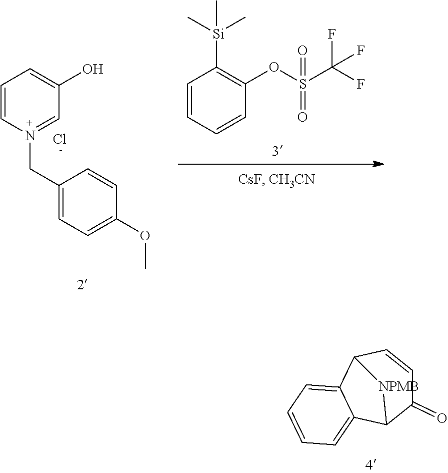

### 12-[(4-Methoxyphenyl)methyl]-12-azatricyclo[6.3.1.0^{2,7}]dodeca-2,4,6,10-tetraen-9-one (4′)

3-Hydroxy-1-(4-methoxyphenyl)methylpyridin-1-ium chloride (50 g, 198 mmol) was dissolved in acetonitrile (300 mL). 2-(trimethylsilyl)phenyltrifluoromethanesulfonate (88.6 g, 297 mmol) was added followed by cesium fluoride (179 g, 1.18 mol). The mixture was stirred at 25° C. for 18 h. The mixture was filtered and the filtrate was concentrated under reduced pressure. The residue was purified by column chromatography (petroleum ether/EtOAc=20/1) to afford 4′ (38.7 g, 132 mmol, 66%) as a brown oil. LCMS: BONB-00122-089-1(ESI) m/z=291.9 (M+H)⁺, t=3.723 min (215 nm).

### Step 3

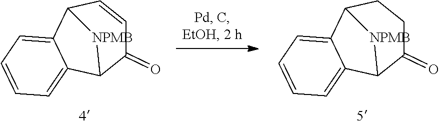

### 12-[(4-Methoxyphenyl)methyl]-12-azatricyclo[6.3.1.0^{2,7}]dodeca-2,4,6-trien-9-one (5′)

12-[(4-Methoxyphenyl)methyl]-12-azatricyclo[6.3.1.0^{2,7}]dodeca-2,4,6,10-tetraen-9-one (23 g, 10.6 mmol) was dissolved in ethyl alcohol (300 mL). Pd/C (5%, 3.5 g) was added, and the reaction mixture was stirred at room temperature under H2 atmosphere for 2 h. LC-MS show that intermediate 4′ was consumed. The mixture was filtered, and the filtrate was concentrated to give the crude product. The crude product was purified by chromatography on silica gel (petroleum ether/EtOAc=10/1) to afford 5′ (21 g, 91%) as an oil. LCMS: BONB-00122-097-1(ESI) m/z=293.9 (M+H)⁺, t=3.172 min (215 nm).

### Step 4

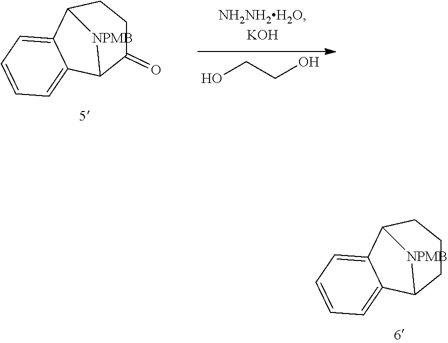

### 12-[(4-Methoxyphenyl)methyl]-12-azatricyclo[6.3.1.0^{2,7}]dodeca-2,4,6-triene (6′)

12-[(4-Methoxyphenyl)methyl]-12-azatricyclo[6.3.1.0^{2,7}]dodeca-2,4,6-trien-9-one (21 g, 40.9 mmol) was dissolved in ethylene glycol (200 mL). Potassium hydroxide (42 g, 408 mmol) and hydrazine hydrate (15 g, 204 mmol) were added, and the reaction mixture was stirred at 190° C. for 4 hour. The mixture was cooled and diluted with water (500 mL). The mixture was extracted with DCM (200 mL×3). The combined organic layers were dried over by Na₂SO₄, filtered and concentrated. The residue was purified by column chromatography on silica gel, eluting with (petroleum ether/EtOAc=20/1) to afford 6′ (8 g, 40%) as oil. LCMS: BONB-00122-099-1(ESI) m/z=279.9 (M+H)⁺, t=3.388 min (215 nm).

### Step 5

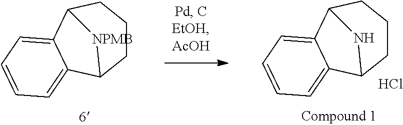

### 12-Azatricyclo[6.3.1.0^{2,7}]dodeca-2,4,6-triene (Compound 1)

12-[(4-Methoxyphenyl)methyl]-12-azatricyclo[6.3.1.0^{2,7}]dodeca-2,4,6-triene (8 g, 17.8 mmol) was dissolved in ethyl alcohol (100 mL). Acetic acid (36% 4 mL) and Pd/C (8 g) were added, and the reaction mixture was stirred overnight at 40° C. under H2 atmosphere. LC-MS show that compound 6 was consumed. The mixture was filtered and the filtrate was concentrated to give the crude product. The crude product was acidified with HCl/MeOH and washed with Et₂O (20 mL), the residue was dried in vacuo to give Compound 1 (4 g, 71%) as a white solid. ¹H NMR (400 MHz, D₂O): δ 7.32-7.17 (m, 4H), 4.81 (d, J=5.1 Hz, 1H), 4.37 (m, 1H), 3.41 (dd, J=17.9.4.8 Hz, 1H), 2.93 (d, J=17.9 Hz, 1H), 2.38-2.23 (m, 2H), 2.15-2.10 (m, 1H), 1.88-1.86 (m, 1H). LCMS: BONB-00112-091(ESI) m/z=160.0 (M+H)⁺, t=1.818 min (214 nm).

### Step 6

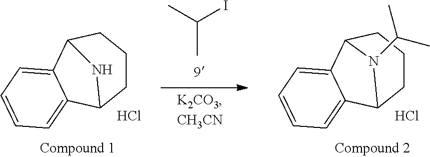

### 12-(Propan-2-yl)-12-azatricyclo[6.3.1.0^{2,7}]dodeca-2,4,6-triene (Compound 2)

12-Azatricyclo[6.3.1.0^{2,7}]dodeca-2,4,6-triene (2.5 g, 15.7 mmol) was dissolved in acetonitrile (100 mL). Potassium carbonate (10.8 g, 78.4 mmol) and 2-iodopropane (7.98 g, 47.0 mmol) were added, and the reaction mixture was stirred at 50° C. overnight. LC-MS show that the reactant was consumed. The mixture was filtered and the filtrate was concentrated. The residue was purified by chromatography on silica gel, eluting with (DCM/MeOH=20/1) to give Compound 2 (2.00 g, 66%) as a white solid. ¹H NMR (400 MHz, D₂O): δ 7.47-7.41 (m, 2H), 7.38 (dd, J=5.5, 3.1 Hz, 2H), 5.13 (s, 2H), 4.93 (s, 1H), 3.14-3.04 (m, 1H), 2.18-2.03 (m, 2H), 1.89 (d, J=14.4 Hz, 2H), 1.65 (dd, J=15.1, 5.8 Hz, 1H), 1.53-1.43 (m, 1H), 1.38 (d, J=6.5 Hz, 1H), 1.25 (d, J=6.6 Hz, 5H). LCMS: BONB-00122-063(ESI) m/z=202.1 (M+H)⁺, t=2.466 min (215 nm).

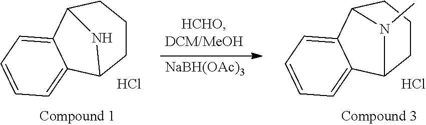

Compound 3

### 12-Methyl-12-azatricyclo[6.3.1.0^{2,7}]dodeca-2,4,6-triene hydrochloride (Compound 3)

12-Azatricyclo[6.3.1.0^{2,7}]dodeca-2,4,6-triene hydrochloride (60 mg, 306 μmol) and 37% HCHO/H₂O (124 μL, 45.9 mg, 1.53 mmol) was dissolved in dichloromethane (6 mL) and methanol (3 mL). The reaction mixture was stirred at 0° C. for 0.5 hour. Sodium triacetoxyborohydride (387 mg, 1.83 mmol) was added, the reaction mixture continued stirring at 25° C. for 1 hours. The mixture was quenched with water (0.2 mL), concentrated and purified by Prep-HPLC, the eluent was acidified with HCl-dioxane (4M, 0.1 mL) and lyophilized overnight to give Compound 3 as a white solid (50 mg, Y: 78%). 1H NMR (400 MHz, D₂O): δ 7.64-7.25 (m, 4H), 4.80 (m, 2H), 2.69 (s, 3H), 2.27-2.01 (m, 2H), 1.88-1.86 (m, 2H), 1.48-1.44 (m, 1H), 0.91-0.86 (m, 1H). LCMS: BONB-00112-119 (ESI) m/z=174.0 (M+H)⁺, t=1.804 min (215 nm).

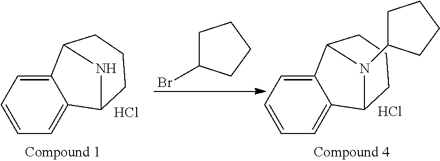

Compound 4

### 12-Cyclopentyl-12-azatricyclo[6.3.1.0^{2,7}]dodeca-2,4,6-triene hydrochloride (Compound 4)

12-Azatricyclo[6.3.1.0^{2,7}]dodeca-2,4,6-triene hydrochloride (60 mg, 306 μmol) and K₂CO₃ (211 mg, 1.53 mmol) were dissolved in ACN (5 mL). Bromocyclopentane (228 mg, 1.53 mmol) was added to the reaction mixture and stirred at 70° C. for 16 hours. The mixture was filtered, concentrated and purified by Prep-HPLC, the eluent was acidified with HCl-dioxane (4M, 0.1 mL) and lyophilized overnight to give Compound 4 as a white solid (40 mg, Y: 49%). ¹H NMR (400 MHz, D₂O): δ 7.37-7.30 (m, 4H), 4.90 (m, 2H), 3.21-3.18 (m, 1H), 2.14-1.95 (m, 4H), 1.80-1.78 (m. 2H), 1.63-1.53 (m, 4H), 1.40-1.38 (m. 3H), 0.90-0.80 (m, 1H). LCMS: BONB-00112-139A1 (ESI) m/z=227.9 (M+H)⁺, t=3.021 min (215 nm).

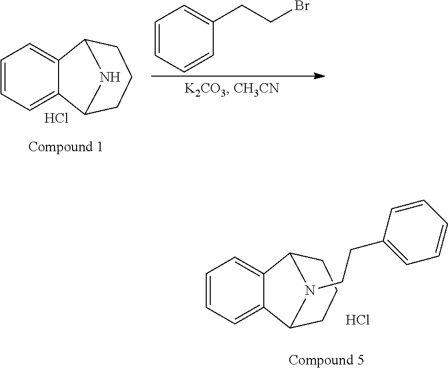

Compound 5

### 12-(2-Phenylethyl)-12-azatricyclo[6.3.1.0^{2,7}]dodeca-2,4,6-triene (Compound 5)

12-Azatricyclo[6.3.1.0^{2,7}]dodeca-2,4,6-triene hydrochloride (200 mg, 1.25 mmol) was dissolved in CH₃CN (6 mL). Potassium carbonate (1.75 g, 12.5 mmol) and (2-bromoethyl)benzene (1.15 g, 6.25 mmol) were added, and the mixture was stirred at 50° C. overnight. The mixture was filtered and the filtrate was concentrated. The residue was purified by HPLC to give Compound 5 (80 mg, 30%) as a white solid. ¹H NMR (400 MHz, D₂O) δ 7.59-7.15 (m, 9H), 5.01 (s, 2H), 3.17 (dd, J=51.6, 8.9 Hz, 4H), 2.20 (t, J=9.7 Hz, 2H), 1.93 (d, J=9.9 Hz, 2H), 1.62-1.46 (m, 1H), 0.97 (dt, J=19.7, 9.5 Hz, 1H). LCMS: OAK-0011410-LCMS(ESI) m/z=264.1 (M+H)⁺, t=3.478 min (215 nm).

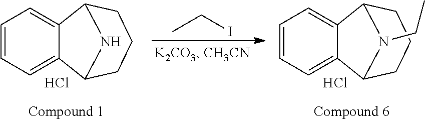

Compound 6

### 12-Ethyl-12-azatricyclo[6.3.1.0,]dodeca-2,4,6-triene hydrochloride (Compound 6)

12-Azatricyclo[6.3.1.0^{2,7}]dodeca-2,4,6-triene hydrochloride (150 mg, 766 μmol) was dissolved in acetonitrile (20 mL). Potassium carbonate (316 mg, 2.29 mmol) and ethyl iodide (143 mg, 919 μmol) were added and the reaction mixture was stirred at 40° C. for 18 h. LCMS show that Compound 1 was consumed. The reaction mixture was filtered and the filtrate was concentrated. The residue was purified by HPLC to give Compound 6 (83.0 mg 40%) as a white solid. ¹H NMR (400 MHz, D₂O) δ 7.43 (ddd, J=21.7, 5.5, 3.2 Hz, 4H), 4.94 (t, J=2.7 Hz, 2H), 2.97 (q, J=7.3 Hz, 2H), 2.19-2.06 (m. 2H), 1.93-1.83 (m, 2H), 1.49 (dt, J=14.4, 5.5 Hz, 1H), 1.24 (t, 1=7.3 Hz, 3H), 0.99-0.83 (m, 1H). LCMS: BONB-00113-141(ESI) m/z=188.1 (M+H)⁺, t=2.416 min (215 nm).

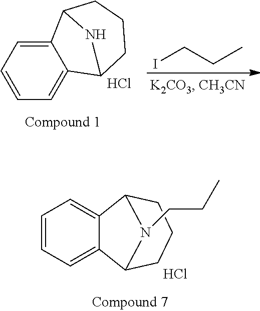

Compound 7

### 12-Propyl-12-azatricyclo[6.3.1.0^{2,7}]dodeca-2,4,6-triene hydrochloride (Compound 7)

12-Azatricyclo[6.3.1.0^{2,7}]dodeca-2,4,6-triene hydrochloride (150 mg, 0.77 mmol) was dissolved in acetonitrile (10 mL). 1-Iodopropane (392 mg, 2.30 mmol) and potassium carbonate (318 mg, 2.30 mmol) were added and the reaction mixture was stirred at 50° C. for 18 h. The reaction solution was filtered and concentrated. The residue was purified by prep-HPLC to afford Compound 7 (52 mg, 0.22 mmol, 29%) as a white solid. ¹H NMR (400 MHz, D₂O) δ 7.47-7.38 (m, 4H), 2.86 (dd, J=9.8, 6.8 Hz, 2H), 2.13 (ddd, J=12.5, 7.7, 2.8 Hz, 2H), 1.86 (d, J=13.6 Hz, 2H), 1.71-1.61 (m, 2H), 1.47 (dd, J=12.8, 6.9 Hz, 2H), 1.02-0.84 (m, 2H), 0.81 (t, J=7.4 Hz, 3H). LCMS: BONB-00125-014 (ESI) m/z=202.1 (M+H)⁺, t=2.749 min (215 nm).

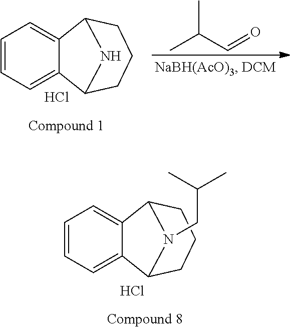

Compound 8

### 12-(2-Methylpropyl)-12-azatricyclo[6.3.1.0^{2,7}]dodeca-2,4,6-triene (Compound 8)

12-Azatricyclo[6.3.1.0^{2,7}]dodeca-2,4,6-triene (150 mg, 942 μmol) was dissolved in DCM (10 mL). 2-Methylpropanal (339 mg, 4.71 mmol) was added, and the reaction mixture was stirred at room temperature for 3 h. Sodium triacetoxyborohydride (998 mg, 4.71 mmol) was added to the mixture and stirred at room temperature overnight. LC-MS show that the reactant was consumed. The mixture was quenched by saturated sodium bicarbonate solution (20 mL) and extracted with DCM (20 mL×3). The combined organic layers were dried by Na₂SO₄, filtered and concentrated. The residue was purified by prep-HPLC to afford Compound 8 (40.0 mg, 185 μmol, 19%) as a white solid. ¹H NMR (400 MHz, D₂O) δ 7.56-7.26 (m, 4H), 4.93 (s, 2H), 2.78 (d, J=7.6 Hz, 2H), 2.24-2.12 (m, 2H), 2.07-1.97 (m, 1H), 1.87 (d, J=11.3 Hz, 2H), 1.48 (d, J=14.5 Hz, 1H), 1.02 (d, J=6.7 Hz, 1H), 0.92 (dd, J=15.9, 6.6 Hz, 6H). LCMS: BONB-00122-054 (ESI) m/z=216.1 (M+H)⁺, t=1.950 min (215 nm).

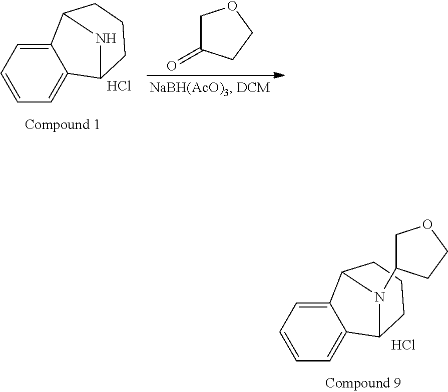 [SMILES: c1ccc2c(c1)C1CCCC2N1 ; Cl ; O=C1CCOC1 ; c1ccc2c(c1)C1CCCC2N1C1CCOC1 ; Cl]

Compound 9

### 12-(Oxolan-3-yl)-12-azatricyclo[6.3.1.0^{2,7}]dodeca-2,4,6-triene hydrochloride (Compound 9)

12-Azatricyclo[6.3.1.0^{2,7}]dodeca-2,4,6-triene hydrochloride (150 mg, 766 μmol), oxolan-3-one (329 mg, 3.83 mmol) and acetic acid (45.9 mg, 766 μmol) were dissolved in DCM (5 mL). The reaction mixture was stirred at 25° C. for 0.5 hour. Sodium triacetoxyborohydride (811 mg, 3.83 mmol) was added to the reaction mixture and stirred at 25° C. for 16 hours. The mixture was quenched with water (0.2 mL), concentrated and purified by Prep-HPLC. The eluent was acidified with HCl-dioxane (4M, 0.1 mL) and lyophilized overnight to give Compound 9 as a white solid (120 mg, Y: 59%). ¹H NMR (400 MHz, D₂O): δ 7.48-7.38 (m, 4H), 5.04-5.03 (m, 1H), 4.91-4.89 (m, 1H), 4.06-3.99 (m, 2H), 3.86-3.83 (m, 1H), 3.74-3.60 (m, 2H), 2.37-2.29 (m, 1H), 2.16-2.06 (m, 3H), 1.91-1.88 (m, 2H), 1.52-1.46 (m, 1H), 0.98-0.89 (m, 1H). LCMS: BONB-00112-145 (ESI) m/z=230.1 (M+H)⁺, t=2.767 min (215 nm).

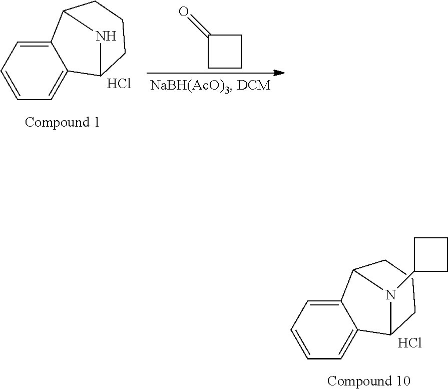 [SMILES: c1ccc2c(c1)C1CCCC2N1 ; Cl ; O=C1CCC1 ; c1ccc2c(c1)C1CCCC2N1C1CCC1 ; Cl]

Compound 10

### 12-Cyclobutyl-12-azatricyclo[6.3.1.0^{2,7}]dodeca-2,4,6-triene hydrochloride (Compound 10)

12-Azatricyclo[6.3.1.0^{2,7}]dodeca-2,4,6-triene hydrochloride (200 mg, 1.02 mmol) was dissolved in methylene chloride (50 mL) and AcOH (0.5 ml). Cyclobutanone (142 mg, 2.04 mmol) was added, and the reaction mixture was stirred at 25° C. for 3 h. Sodium triacetoxyborohydride (648 mg, 3.06 mmol) was added, and the mixture continued stirring at 25° C. for 17 h. The mixture was quenched by saturated sodium bicarbonate solution (20 mL) and extracted with DCM (20 mL×3). The combined organic layers were dried over by Na₂SO₄, filtered and concentrated. The residue was purified by prep-HPLC to afford Compound 10 (130 mg, 60%) as a colorless solid. ¹H NMR (400 MHz, D₂O) δ 7.43 (dt, J=7.5, 3.8 Hz, 2H), 7.39-7.32 (m, 2H), 4.80 (t, J=2.6 Hz, 2H), 3.44 (p, J=8.4 Hz, 1H), 2.23-2.01 (m, 6H), 1.91-1.73 (m, 3H), 1.69-1.55 (m, 1H), 1.46 (dt, J=14.3, 5.4 Hz, 1H), 0.88 (dtt, J=18.9, 12.6, 6.1 Hz, 1H). LCMS: BONB-00113-148(ESI) m/z=214.1 (M+H)⁺, t=2.990 min (215 nm).

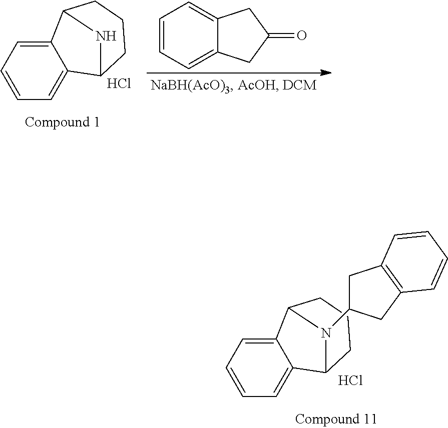

Compound 11

### 12-(2,3-Dihydro-1H-inden-2-yl)-12-azatricyclo[6.3.1.0^{2,7}]dodeca-2,4,6-triene hydrochloride (Compound 11)

12-Azatricyclo[6.3.1.0^{2,7}]dodeca-2,4,6-triene hydrochloride (200 mg, 1.02 mmol) was dissolved in DCM (15 mL) and AcOH (1 mL). 2,3-dihydro-1H-inden-2-one (674 mg, 5.10 mmol) was added and the reaction was stirred at room temperature for 2 h. Sodium triacetoxyborohydride (648 mg, 3.06 mmol) was added, and the mixture continued stirring overnight at room temperature. The mixture was quenched by saturated NaHCO₃ solution (20 mL) and extracted with DCM (20 mL×3). The organic solvent was dried over anhydrous Na₂SO₄, filtered and concentrated. The residue was purified by prep-HPLC to afford Compound 11 (100 mg, 32%) as a white solid. ¹H NMR (400 MHz, D₂O) δ 7.52-7.39 (m, 4H), 7.32-7.13 (m, 4H), 5.03 (d, J=42.9 Hz, 2H), 3.76 (t, J=8.5 Hz, 1H), 3.27 (dd, J=15.8, 8.3 Hz, 2H), 3.14 (dd, J=15.8, 8.6 Hz, 2H), 2.15 (tdd, J=8.2, 5.4, 2.7 Hz, 2H), 1.92 (dd, J=13.6, 4.6 Hz, 2H), 1.56-1.45 (m, 11H), 0.96 (dd, J=13.5, 7.0 Hz, 1H). LCMS: BONB-00123-25 (ESI) m/z=276.1 (M+H)⁺, t=3.683 min (215 nm).

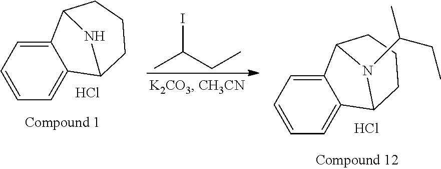

Compound 12

### 12-(Butan-2-yl)-12-azatricyclo[6.3.1.0^{2,7}]dodeca-2,4,6-triene (Compound 12)

12-Azatricyclo[6.3.1.0^{2,7}]dodeca-2,4,6-triene (200 mg, 1.25 mmol) was dissolved in acetonitrile (20 mL). Potassium carbonate (794 mg, 5.74 mmol) and 2-iodobutane (598 mg, 3.24 mmol) were added, and the reaction mixture was stirred at 50° C. overnight. LC-MS show that the reactant was consumed. The mixture was filtered and the filtrate was concentrated. The residue was purified by prep-HPLC to give Compound 12 (70 mg, 22%) as a white solid. ¹H NMR (400 MHz, D₂O) δ 7.44 (dt, J=7.7, 3.9 Hz, 2H), 7.38 (d, J=2.0 Hz, 2H), 5.19-4.92 (m, 2H), 2.94-2.84 (m, 1H), 2.22-2.06 (m, 2H), 1.89 (d, J=11.6 Hz, 1H), 1.76 (dd, J=6.7, 3.8 Hz, 1H), 1.64 (d, J=15.5 Hz, 1H), 1.59-1.43 (m, 2H), 1.29 (dd, J=52.8, 6.5 Hz, 3H), 0.99 (t, J=7.5 Hz, 1H), 0.95-0.79 (m, 3H). LCMS: BONB-00122-058(ESI) m/z=216.2 (M+H)⁺, t=2.954 min (215 nm).

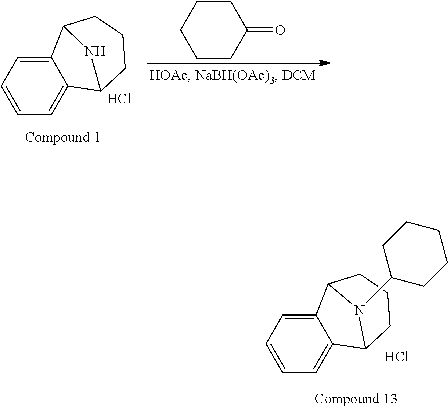 [SMILES: c1ccc2c(c1)C1CCCC2N1 ; Cl ; O=C1CCCCC1 ; c1ccc2c(c1)C1CCCC2N1C1CCCCC1 ; Cl]

Compound 13

### 12-Cyclohexyl-12-azatricyclo[6.3.1.0^{2,7}]dodeca-2,4,6-triene hydrochloride (Compound 13)

To a stirred solution of 12-azatricyclo[6.3.1.0^{2,7}]dodeca-2,4,6-triene hydrochloride (0.15 g, 766 μmol) in methylene chloride (20 mL), was added acetic acid (1.05 g, 17.4 mmol) at RT. After the solution became clear, cyclohexanone (375 mg, 3.83 mmol) was added to the reaction. The mixture was stirred at RT for 5 h. Sodium triacetoxyborohydride (811 mg, 3.83 mmol) was added to the mixture, and the reaction was stirred at RT for 16 h. LCMS showed the reaction was completed. The reaction was quenched by saturated NaHCO₃ and extracted with DCM (15 mL×2). The combined organic layers were dried over anhydrous Na₂SO₄, filtered and concentrated. The residue was purified by prep-HPLC (base) and acidified by 1 drop of 4N HCl/MeOH to give 12-cyclohexyl-12-azatricyclo[6.3.1.0^{2,7}]dodeca-2,4,6-triene hydrochloride (80.0 mg, 37%) as a light yellow solid. ¹H NMR (400 MHz, D₂O) δ 7.40 (d, J=23.9 Hz, 4H), 5.07 (d, J=86.4 Hz, 2H), 3.73-2.78 (m, 1H), 2.32-1.95 (m, 4H), 1.95-1.58 (m, 4H), 1.41 (d, J=84.3 Hz, 4H), 0.98 (d, J=48.8 Hz, 4H). LCMS: BONB-00111-129-P (ESI) m/z=242.1 (M+H)⁺, t=1.534 min (215 nm).

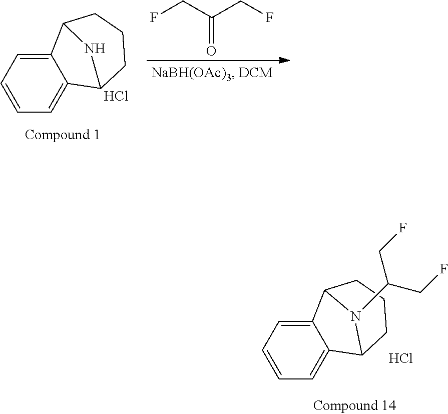

Compound 14

### 12-(1,3-Difluoropropan-2-yl)-12-azatricyclo[6.3.1.0²0.7]dodeca-2,4,6-triene hydrochloride (Compound 14)

12-Azatricyclo[6.3.1.0^{2,7}]dodeca-2,4,6-triene hydrochloride (180 mg, 919 μmol), 1,3-difluoropropan-2-one (431 mg, 4.59 mmol) were dissolved in DCM (8 mL). The reaction mixture was stirred at 25° C. for 0.5 hour. Sodium triacetoxyborohydride (972 mg, 4.59 mmol) was added to the reaction mixture and stirred at 25° C. for 48 hours. The mixture was quenched with water (0.3 mL), concentrated and purified by Pre-HPLC. The eluent was acidified with HCl-dioxane (4M, 0.1 mL) and lyophilized overnight to give Compound 14 as a white solid (90 mg, Y: 36%). ¹H NMR (400 MHz, D₂O): δ 7.49-7.41 (m, 4H), 5.25 (m, 2H), 4.95-4.79 (m, 4H), 3.60 (m, 1H), 2.28-2.21 (m, 2H), 1.92 (m, 2H), 1.54-1.49 (m, 1H), 0.99-0.93 (m, 1H). LCMS: BONB-00112-152 (ESI) m/z=238.1 (M+H)⁺, t=3.178 min (215 nm).

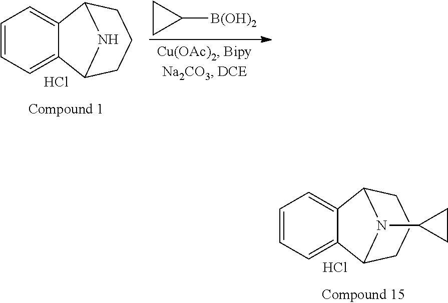

Compound 15

### 12-Cyclopropyl-12-azatricyclo[6.3.1.0²0.7]dodeca-2,4,6-triene hydrochloride (Compound 15)

12-Azatricyclo[6.3.1.0^{2,7}]dodeca-2,4,6-triene hydrochloride (500 mg, 3.14 mmol) was dissolved in ethylene dichloride (15 mL). Cyclopropylboronic acid (539 mg, 6.28 mmol), 2,2′-bipyridine (490 mg, 3.14 mmol) sodium carbonate (678 mg, 6.28 mmol) and (acetyloxy)cuprio acetate (570 mg, 3.14 mmol) were added, and the mixture was heated to reflux overnight. The mixture was filtered and the filtrate was concentrated under reduced pressure. The residue was purified by column chromatography on silica gel, eluting with (PE:EA=4:1) to give Compound 15 (200 mg, 39%) as a white solid. ¹H NMR (400 MHz, D₂O) δ 7.59-7.39 (m, 4H), 5.11 (s, 2H), 2.66 (s, 1H), 2.21-1.84 (m, 4H), 1.51 (dt, J=11.2, 8.3 Hz, 1H). 1.15-0.76 (m, 5H). LCMS: OAK-0011410-LCMS(ESI) m/z=199.9 (M+H)⁺, t=3.360 min (215 nm).

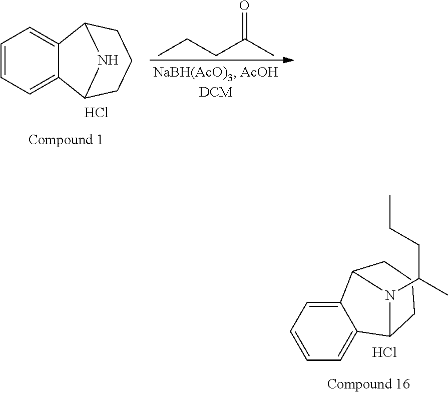

Compound 16

### 12-(Pentan-2-yl)-12-azatricyclo[6.3.1.0²0.7]dodeca-2,4,6-triene hydrochloride (Compound 16)

12-Azatricyclo[6.3.1.0^{2,7}]dodeca-2,4,6-triene hydrochloride was dissolved in DCM (20 mL). Pentan-2-one (538 mg, 6.25 mmol) was added, and the reaction mixture was stirred at RT for 3 h. Sodium triacetoxyborohydride (794 mg, 3.75 mmol) was added to the mixture and stirred at RT overnight. LC-MS show that the reactant was consumed. The mixture was quenched by saturated NaHCO₃ solution (20 mL) and extracted with DCM (20 mL×3). The combined organic layers were dried by Na₂SO₄, filtered and concentrated. The residue was purified by prep-HPLC to afford Compound 16 (70.0 mg, 24%) as a white solid. ¹H NMR (400 MHz, D₂O) δ 7.44 (dd, J=5.4, 3.1 Hz, 2H), 7.38 (d, J=3.4 Hz, 2H), 5.14 (s, 2H), 2.93 (s, 1H), 2.11 (s, 2H), 1.89 (d, J=14.1 Hz, 2H), 1.66 (s, 2H), 1.48 (d, J=14.3 Hz, 3H), 1.35 (d, J=6.6 Hz, 11H), 1.21 (t, J=9.0 Hz, 3H), 0.93 (dd, J=15.1, 7.9 Hz, 2H), 0.80 (t, J=7.3 Hz, 2H). LCMS: BONB-00122-084(ESI) m/z=230.0 (M+H)⁺, t=3.315 min (215 nm).

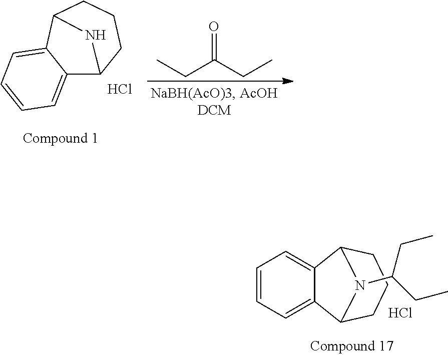

Compound 17

### 12-(Pentan-3-yl)-12-azatricyclo[6.3.1.0^{2,7}]dodeca-2,4,6-trienehydrochloride (Compound 17)

12-Azatricyclo[6.3.1.0^{2,7}]dodeca-2,4,6-triene hydrochloride (150 mg, 0.77 mmol) was dissolved in DCM (10 mL). Pentan-3-one (331 mg, 3.85 mmol) and AcOH (0.2 mL) were added, and the mixture stirred at 25° C. for 2 h. Sodium triacetoxy borohydride (812 mg, 3.85 mmol) was added, and the reaction mixture was stirred at 25° C. for 16 h. The reaction solution was filtered and concentrated, and the residue was purified by prep-HPLC to afford Compound 17 (45 mg, 22%) as a white solid. ¹H NMR (400 MHz, D₂O) δ 7.42 (d, J=15.7 Hz, 4H), 5.12 (s, 2H), 2.72 (s, 1H), 2.25-1.42 (m, 10H), 0.87 (dd, J=25.5, 18.0 Hz, 6H). LCMS: BONB-00125-035(ESI) m/z=230.0 (M+H)⁺, t=3.555 min (215 nm).

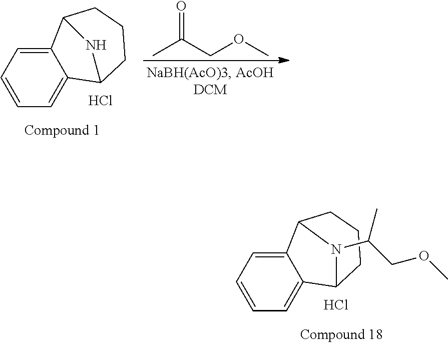

Compound 18

### 12-(1-Methoxypropan-2-yl)-12-azatricyclo[6.3.1.0^{2,7}]dodeca-2,4,6-triene (Compound 18)

12-Azatricyclo[6.3.1.0^{2,7}]dodeca-2,4,6-triene hydrochloride was dissolved in DCM (50 mL). 1-Methoxy-2-propanone (1.10 g, 12.5 mmol) was added, and the reaction mixture was stirred at RT for 3 h. Sodium triacetoxyborohydride (1.32 g, 6.25 mmol) was added to the mixture and the reaction was stirred at RT overnight. LC-MS show that the reactant was consumed. The mixture was quenched by saturated NaHCO₃ solution (50 mL) and extracted with DCM (50 mL×₃). The combined organic layers were dried over by Na₂SO₄, filtered and concentrated. The residue was purified by prep-HPLC to afford Compound 18 (76.0 mg, 26%) as a white solid. ¹H NMR (400 MHz, D₂O) δ 7.48-7.42 (m, 2H), 7.41-7.34 (m, 2H), 5.22-4.87 (m, 3H), 3.84 (dd, J=11.4, 3.1 Hz, 1H), 3.66 (dd, J=11.3, 2.9 Hz, 1H), 3.55 (ddd, J=16.7, 11.8, 4.0 Hz, 2H), 3.38 (s, 1H), 3.26 (s, 2H), 3.20 (dd, J=14.3, 7.1 Hz, 1H), 2.24-2.09 (m, 2H), 1.90 (dd. J=17.6, 11.4 Hz, 2H), 1.74-1.61 (m, 1H), 1.48 (dd, J=13.8, 5.8 Hz, 1H), 1.35 (dd, J=54.4, 6.8 Hz, 3H), 1.01-0.84 (m, 1H). LCMS: BONB-00122-093(ESI) m/z=231.9 (M+H)⁺, t=2.818 min (215 nm).

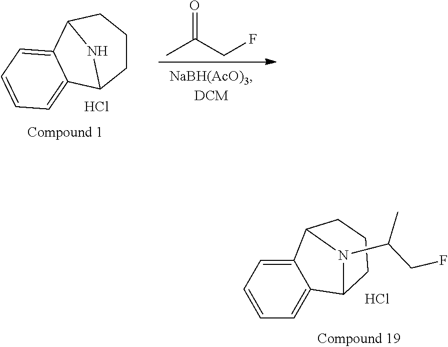

Compound 19

### 12-(1-Fluoropropan-2-yl)-12-azatricyclo[6.3.1.0^{2,7}]dodeca-2,4,6-triene hydrochloride (Compound 19)

12-Azatricyclo[6.3.1.0^{2,7}]dodeca-2,4,6-triene hydrochloride was dissolved in DCM (15 mL). 1-Fluoropropan-2-one (387 mg, 5.10 mmol) was added, and the mixture was stirred for 2 h at room temperature. Sodium triacetoxyborohydride (1.18 g, 5.61 mmol) was added, and the resulting mixture continued stirring at room temperature overnight. The mixture was quenched by saturated NaHCO₃ solution (50 mL) and extracted with DCM (50 mL×3). The combined organic layers were dried over by Na₂SO₄, filtered and concentrated. The residue was purified by prep-HPLC to afford Compound 19 (70 mg, 27%) as a white solid. ¹H NMR (400 MHz, D₂O) δ 7.45 (dd, J=8.1, 5.0 Hz, 2H), 7.41 (s, 2H), 5.27-4.76 (m. 3H), 4.65-4.41 (m, 1H), 3.49-3.11 (m, 1H), 2.19 (dd, J=12.3, 9.9 Hz, 2H), 1.81 (dd, J=82.9, 10.0 Hz, 2H), 1.56 (t, J=35.9 Hz, 1H), 1.48-1.25 (m, 3H), 1.02-0.84 (m, 1H). LCMS. BONB-00123-75 (ESI) m/z=219.9 (M+H)⁺, t=3.088 min (215 nm).

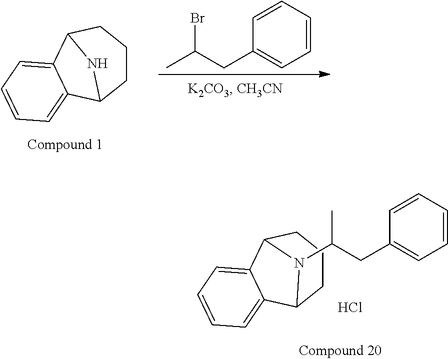

Compound 20

### 10-(1-Phenylpropan-2-yl)-10-azatricyclo[6.3.1.0^{2,7}]dodeca-2,4,6-triene hydrochloride (Compound 20)

10-Azatricyclo[6.3.1.0^{2,7}]dodeca-2,4,6-trienehydrochloride (150 mg, 766 μmol) was dissolved in acetonitrile (30 mL). Potassium carbonate (321 mg, 2.29 mmol) and (2-bromopropyl)benzene (455 mg, 2.29 mmol) were added, and the mixture was stirred at 100° C. for 19 h. The reaction mixture was filtered and the filtrate was concentrated. The residue was purified by prep-HPLC to afford Compound 20 as a colorless solid (150 mg, 21%). ¹H NMR (400 MHz, D₂O) δ 7.50-7.06 (m, 9H), 5.15 (dd, J=68.6, 41.2 Hz, 2H), 3.47-3.16 (m, 2H), 2.78-2.63 (m, 1H), 2.38-2.08 (m, 2H), 1.98-1.62 (m, 2H), 1.60-1.44 (m, 1H), 1.21 (dd, J=50.3, 6.6 Hz, 3H), 0.92 (ddd, J=26.9, 12.7, 6.3 Hz, 1H). LCMS: BONB-00113-180 (EST) m/z=277.9 (M+H)⁺, t=3.608 min (215 nm).

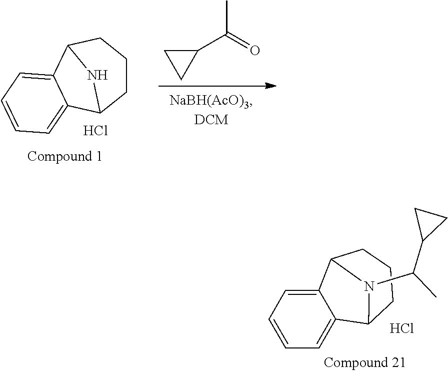

Compound 21

### 12-(1-Cyclopropylethyl)-12-azatricyclo[6.3.1.0]dodeca-2,4,6-triene hydrochloride (Compound 21)

To a solution of 12-azatricyclo[6.3.1.0^{2,7}]dodeca-2,4,6-triene hydrochloride (400 mg, 2.04 mmol) in methylene chloride (15 mL) was added 1-cyclopropylethan-1-one (856 mg, 10.2 mmol). The mixture was stirred for 2 h at room temperature. Sodium triacetoxyborohydride (2.37 g, 11.22 mmol) was added and the resulting mixture was stirred overnight. The mixture was quenched by saturated NaHCO₃ solution (50 mL) and extracted with DCM (50 mL×3). The solvent was dried over anhydrous Na₂SO₄, filtered, and concentrated. The residue was purified by prep-HPLC to afford Compound 21 (45 mg, 8%) as a white solid. ¹H NMR (400 MHz, D₂O) δ 7.26 (d, J=22.0 Hz, 4H), 5.48 (s, 1H), 4.93 (s, 1H), 2.09 (s, 1H), 2.00 (s, 2H), 1.78 (s, 2H), 1.42 (d, J=65.5 Hz, 1H), 1.21 (d, J=47.8 Hz, 3H), 0.74 (s, 2H), 0.50 (d, J=49.7 Hz, 2H), −0.07 (d, J=56.9 Hz, 2H). LCMS: BONB-00123-72 (ESI) m/z=228.0 (M+H)⁺, t=2.848 min (215 nm).

Compound 22

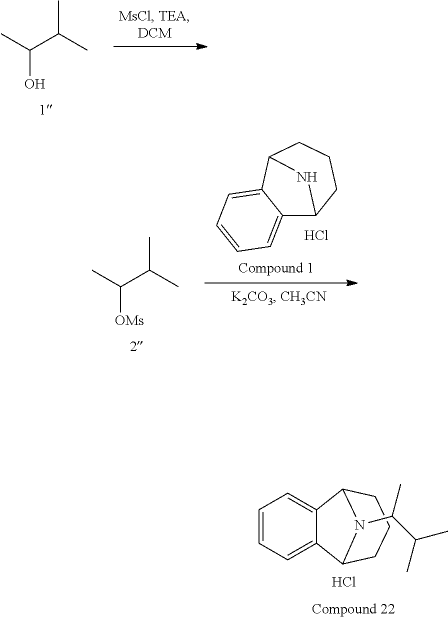

Step 1

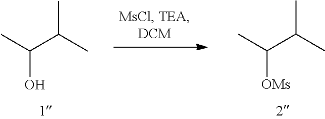

### 3-Methylbutan-2-yl methanesulfonate (2″)

3-Methylbutan-2-ol (5 g, 56.7 mmol) and triethylamine (11.4 g, 113 mmol) were dissolved in DCM (20 mL). Methanesulfonyl chloride (9.73 g, 85.0 mmol) was added dropwise at 0° C. under nitrogen atmosphere. The reaction mixture was stirred at room temperature overnight. The mixture was poured into water (20 mL), and extracted with DCM (20 mL×3). The combined organic layers were dried over sodium sulfate, filtered and concentrated. The residue was purified by column chromatography to afford 2″ (6 g, 64%) as a colorless oil. ¹H NMR (400 MHz, CDCl₃) δ 4.62 (dd, J=6.3, 5.5 Hz, 1H), 2.99 (s, 3H), 1.90 (tt, J=12.2, 3.4 Hz. 1H), 1.37 (d, J=6.4 Hz, 3H), 0.96 (dd, J=6.8, 2.5 Hz, 6H).

Step 2

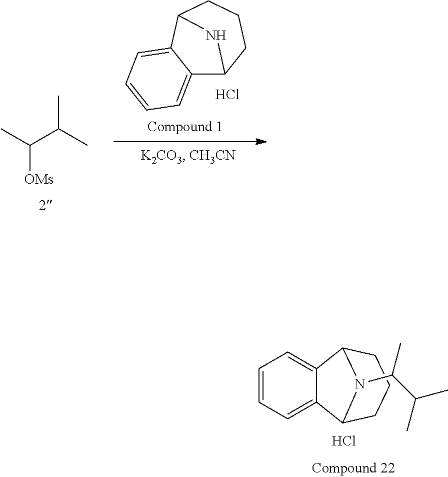

### 12-(3-Methylbutan-2-yl)-12-azatricyclo[6.3.1.0^{2,7}]dodeca-2,4,6-triene hydrochloride (Compound 22)

12-Azatricyclo[6.3.1.0^{2,7}]dodeca-2,4,6-triene hydrochloride (200 mg, 1.02 mmol) was dissolved in acetonitrile (10 mL). Potassium carbonate (281.9 mg, 2.04 mmol) and 3-methylbutan-2-yl methanesulfonate (1 g, 6.02 mmol) were added, and the reaction mixture was stirred at 80° C. overnight. The reaction solution was filtered and the filtrate was concentrated. The residue was purified by prep-HPLC to afford Compound 22 (53 mg, 20%) as a white solid. ¹H NMR (400 MHz, D₂O) δ 7.51-7.33 (m, 4H), 5.06 (dd, J=83.4, 24.4 Hz, 2H). 3.33-2.67 (m, 1H), 2.42-2.08 (m, 3H), 1.99-1.57 (m, 2H), 1.54-1.20 (m, 3H), 1.21-0.95 (m, 2H), 0.91 (t, J=6.1 Hz, 3H), 0.80 (t, J=25.7 Hz, 3H). LCMS: BONB-00123-126 (ESI) m/z=230.2 (M+H)⁺, t=3.019 min (215 nm).

Compound 23 and Compound 24

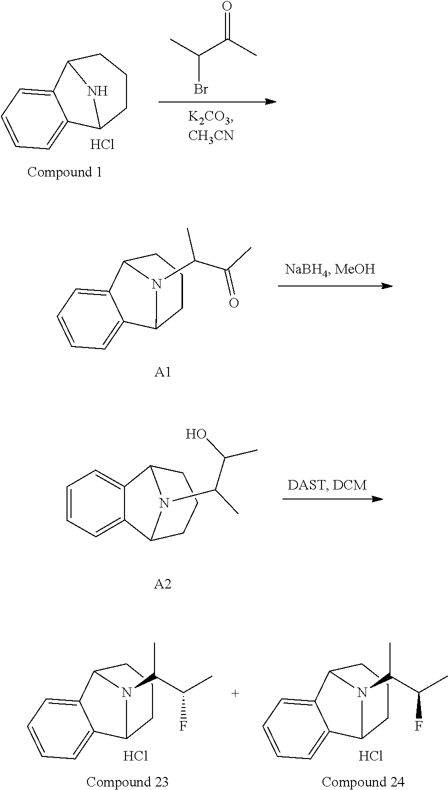

Step 1

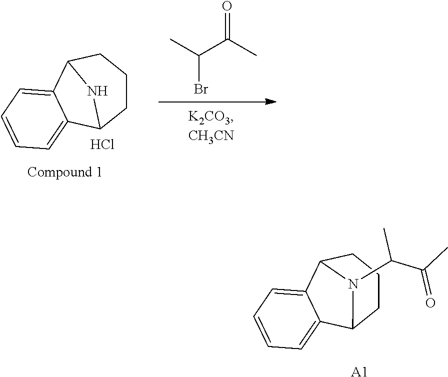

### 3-{12-Azatricyclo[6.3.1.0^{2,7}]dodeca-2,4,6-trien-12-yl}butan-2-one (A1)

12-Azatricyclo[6.3.1.0^{2,7}]dodeca-2,4,6-triene hydrochloride (500 mg, 2.55 mmol) was dissolved in acetonitrile (50 mL). Potassium carbonate (704 mg. 5.10 mmol) and 3-bromobutan-2-one (576 mg, 3.82 mmol) were added, and the reaction mixture was stirred at 60° C. for 18 h. The reaction mixture was filtered and concentrated to afford A1 as an oil (570 mg, 97.3%). LCMS: BONB-00113-197(ESI) m/z=229.9 (M+H)⁺, t=3.149 min (215 nm).

Step 2

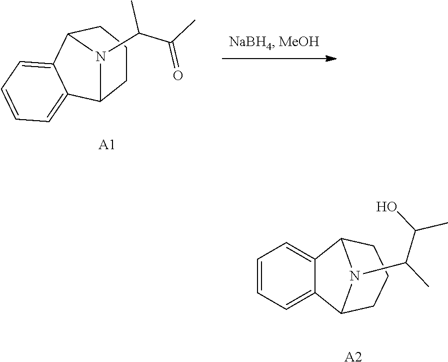

### 3-{12-Azatricyclo[6.3.1.0?⁷]dodeca-2,4,6-trien-12-yl}butan-2-ol (A2)

3-{12-Azatricyclo[6.3.1.0^{2,7}]dodeca-2,4,6-trien-12-yl}butan-2-one (570 mg, 2.48 mmol) was dissolved in methanol (30 mL). Sodium borohydride (281 mg, 7.44 mmol) was added, and the reaction mixture was stirred at room temperature for 17 h. The reaction mixture was concentrated. The residue was dissolved in DCM (20 mL), washed with brine, dried over Na₂SO₄, filtered and concentrated to afford A2 as an oil (560 mg, 97.6%). LCMS: BONB-00113-198(ESI) m/z=231.9 (M+H)⁺, t=1.856&2.229 min (215 nm).

Step 3

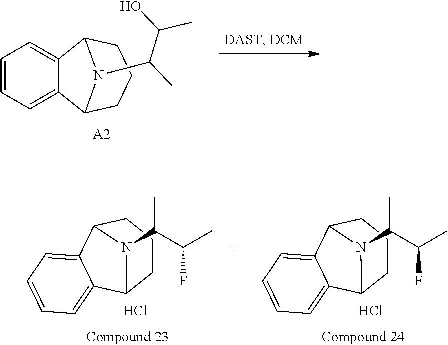

### 12-(3-Fluorobutan-2-yl)-12-azatricyclo[6.3.1.0^{2,7}]dodeca-2,4,6-triene hydrochloride (Compound 23 and Compound 24)

3-{12-Azatricyclo[6.3.1.0^{2,7}]dodeca-2,4,6-trien-12-yl}butan-2-ol (500 mg, 2.16 mmol) was dissolved in methylene chloride (100 mL). Diethyl(trifluoro-lambda4-sulfanyl)amine (3.48 g, 21.6 mmol) was added at −70° C. The reaction mixture was stirred at −70° C. for 3 h and at rt for 2d under N₂ atmosphere. The mixture was quenched by saturated NaHCO₃ solution and extracted with DCM (30 mL×3). The combined solutions were washed with brine, dried over Na₂SO₄, filtered and concentrated. The residue was purified by prep-HPLC to afford Compound 23 (105 mg, 18%) as a white solid. ¹H NMR (400 MHz, D₂O) δ 7.54-7.32 (m, 4H), 5.27 (s, 1H), 5.15 (s, 2H), 3.48-2.92 (m, 1H), 2.17 (dd, J=12.4, 10.1 Hz, 2H), 2.00-1.64 (m, 2H), 1.62-0.77 (m, 8H). ¹⁹F NMR (376 MHz, D₂O) δ −191.82 (s). LCMS: BONB-00113-201-1 (EST) m/z=233.9 (M+H)⁺, t=2.723 min (215 nm).

Compound 24 (110 mg, 19%) was afforded as a white solid. ¹H NMR (400 MHz, D₂O) δ 7.43 (d, J=23.2 Hz, 4H), 5.17 (s, 2H), 4.95 (d, J=50.3 Hz, 2H), 3.33 (s, 1H), 2.21 (d, J=11.4 Hz, 2H), 1.90 (s, 2H), 1.19 (ddd, J=69.7, 33.8, 10.1 Hz, 8H). ¹⁹F NMR (376 MHz, D₂O) δ −173.56 (s). LCMS: BONB-00113-201-2(ESI) m/z=233.9 (M+H)⁺, t=2.851 min (215 nm).

Compound 25

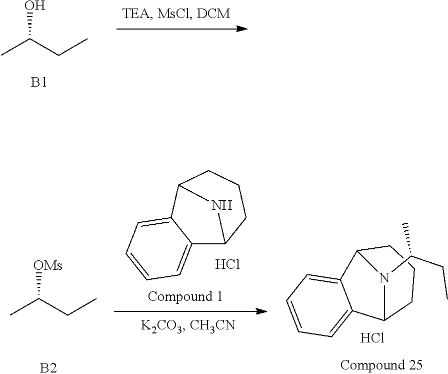

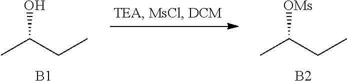

### (2S)-Butan-2-yl methanesulfonate (B2)

(2S)-Butan-2-ol (500 mg, 6.74 mmol) was dissolved in methylene chloride (7 mL). Methanesulfonyl chloride (1.53 g, 13.4 mmol) and triethylamine (3 mL, 20.1 μmol) were added, and the reaction mixture was stirred at room temperature for 7 h. The reaction mixture was diluted with water, extracted with DCM (15 mL×3). The combined organic layers were dried with Na₂SO₄, filtered and concentrated to give B2 (1 g, 97%) as a colorless oil, and it was used to next step without further purification. ¹H NMR (400 MHz, CDCl3) δ 4.75 (dd, J=12.4, 6.2 Hz, 1H), 2.98 (d, J=18.8 Hz, 3H), 1.79-1.62 (m, 2H), 1.44-1.36 (m, 3H), 1.03-0.91 (m, 3H).

Step 2

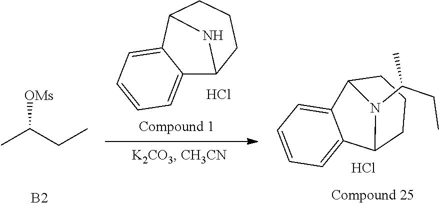

### 12-[(2R)-Butan-2-yl]-12-azatricyclo[6.3.1.0^{2,7}]dodeca-2,4,6-triene hydrochloride (Compound 25)

12-Azatricyclo[6.3.1.0^{2,7}]dodeca-2,4,6-triene hydrochloride (208 mg, 1.31 mmol) was dissolved in acetonitrile (20 mL). Potassium carbonate (4.53 g, 32.8 mmol) and (2S)-butan-2-yl methanesulfonate (1 g, 6.56 mmol) were added, and the reaction mixture was stirred at 50° C. for 2 d. The reaction was filtered and concentrated. The residue was purified by Prep-HPLC to give Compound 25 ((150 mg, 9%) as a colorless solid. ¹H NMR (400 MHz, D₂O) δ 7.57-7.30 (m, 4H), 5.21-4.92 (m, 2H), 2.88 (dd, J=7.9, 5.0 Hz, 1H), 2.25-2.03 (m, 2H), 1.89 (d, J=12.1 Hz, 2H), 1.84-1.70 (m, 1H), 1.69-1.57 (m, 1H), 1.57-1.40 (m, 2H), 1.29 (dd, J=53.7, 6.5 Hz, 3H), 1.03-0.75 (m, 4H). LCMS: BONB-00121-111(ESI) m/z=216.2 (M+H)⁺, t=2.344 min (215 nm).

Compound 26

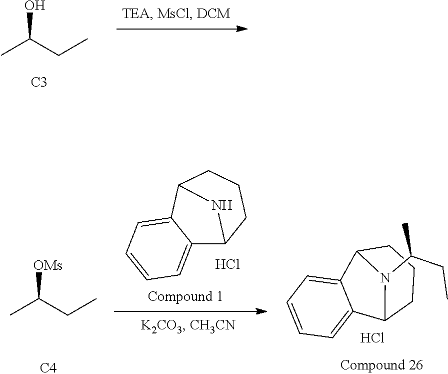

Step 1

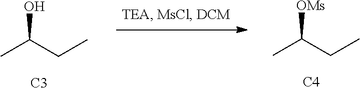

### (2R)-sec-butyl methanesulfonate (C4)

(2R)-Butan-2-ol (1 g, 13.5 mmol) was dissolved in methylene chloride (20 mL). Methanesulfonyl chloride (1.85 g, 16.2 mmol) and triethylamine (2.73 g, 27 mmol) were added, and the reaction mixture was stirred at room temperature for 7 h. The reaction mixture was diluted with water, and extracted with DCM (15 mL×3). The combined organic layers were dried with Na₂SO₄, filtered and concentrated to give C4 (1.1 g, 54%) as a colorless oil, and it was used to next step without further purification. ¹H NMR (400 MHz, CDCl3) δ 4.74 (d, J=6.1 Hz, 1H), 3.00 (s, 3H), 1.89-1.63 (m, 2H), 1.41 (d, J=6.3 Hz, 3H), 0.99 (t, J=7.4 Hz, 3H).

Step 2

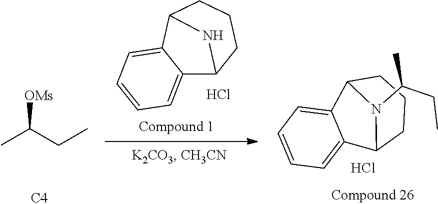

### 12-[(2S)-Butan-2-yl]-12-azatricyclo[6.3.1.0^{2,7}]dodeca-2,4,6-triene hydrochloride (Compound 26)

12-Azatricyclo[6.3.1.0^{2,7}]dodeca-2,4,6-triene hydrochloride (200 mg, 1.02 mmol) was dissolved in acetonitrile (10 mL). Potassium carbonate (281.9 mg, 2.04 mmol) and 3-methylbutan-2-yl methanesulfonate (1 g, 6.02 mmol) were added, and the reaction mixture was stirred at 80° C. overnight. The reaction solution was filtered and concentrated. The residue was purified by prep-HPLC to afford Compound 26 (60 mg, 23%) as a white solid. ¹H NMR (400 MHz, D₂O) δ 7.57-7.28 (m, 4H), 5.32-4.89 (m, 2H), 2.90 (dd, J=19.8, 13.3 Hz, 1H), 2.22-2.05 (m, 2H), 1.95-1.77 (m, 2H), 1.77-1.55 (m. 1H), 1.50 (td, J=14.6, 7.1 Hz, 2H), 1.28 (dd, J=53.2, 6.5 Hz, 3H), 1.16-0.92 (m, 1H), 0.91-0.77 (m, 3H). LCMS: BONB-00123-121 (ESI) m/z=216.2 (M+H)⁺, t=2.422 min (215 nm).

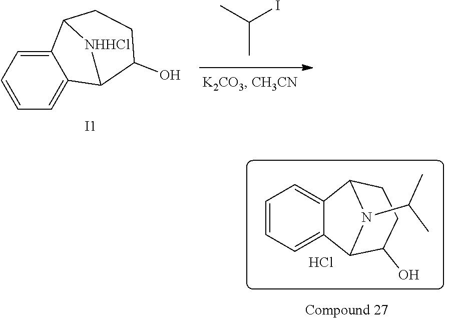

### 12-(propan-2-yl)-12-azatricyclo[6.3.1.0^{2,7}]dodeca-2,4,6-trien-9-ol hydrochloride (Compound 27)

To a stirred solution of compound 11 (200 mg, 0.94 mmol) and K2C03 (391 mg, 1.89 mmol) in ACN (15 mL) was added 2-iodopropane (0.2 mL, 2.83 mmol) at RT. The mixture was stirred at 50° C. for 8 h in a sealed tube. LCMS showed the reaction was completed. The reaction was concentrated and extracted with EtOAc (50 mL*2). The combined organic layers were dried over anhydrous Na₂SO₄, filtered and concentrated. The residue obtained was purified by prep-HPLC (base) and acidified by 1 drop of 4N HCl/MeOH to give Compound 27 (120 mg, 50%) as a light yellow solid. ¹H NMR (400 MHz, D₂O) δ 7.53-7.36 (m, 4H), 5.08 (d, J=66.2 Hz, 1H), 4.86 (dd. J=50.1, 3.3 Hz, 1H), 4.41-4.19 (m, 1H), 3.48 (ddd, J=26.3, 12.9, 6.5 Hz, 1H), 2.08 (dddd, J=27.8, 12.7, 5.4, 2.9 Hz, 1H), 1.96-1.72 (m, 2H), 1.33 (ddd, J=17.2, 8.3, 4.1 Hz, 6H), 0.68 (ddd, J=24.0, 12.9, 6.6 Hz, 1H). LCMS: CP-0023226-014-P (ESI) m/z=218.1 (M+H)⁺, t=2.434 min (215 nm).

Compound 28

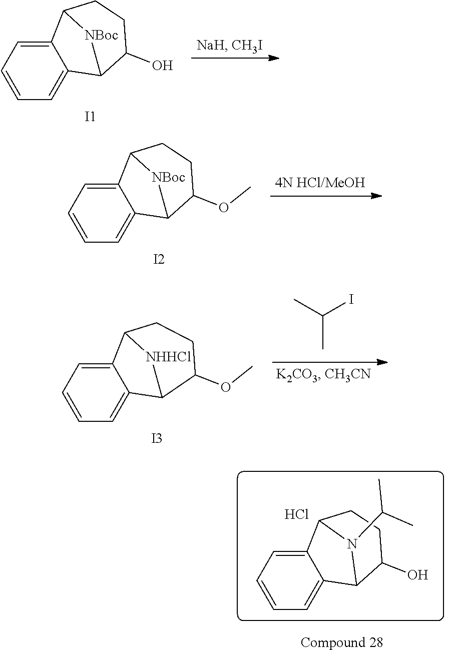

Step 1

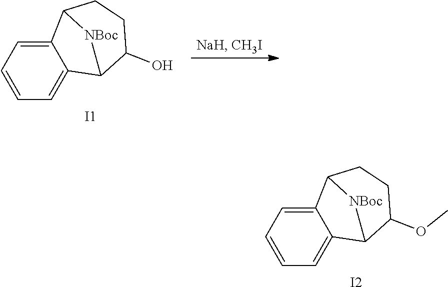

### Tert-butyl 9-methoxy-12-azatricyclo[6.3.1.0^{2,7}]dodeca-2,4,6-triene-12-carboxylate (12)

To a stirred solution of sodium hydride (723 mg, 18.1 mmol) in THF (10 mL) was added tert-butyl 9-hydroxy-12-azatricyclo[6.3.1.0^{2,7}]dodeca-2,4,6-triene-12-carboxylate (0.5 g, 1.81 mmol) in a THF (5 mL) at 0° C. under N₂. The mixture was stirred at RT for 15 min. Iodomethane (2.56 g, 18.1 mmol) was added and the mixture was stirred at RT for 2 h. LCMS showed the reaction was completed. The mixture was poured to aq. NH₄Cl (20 mL). The mixture was extracted with EA (2×40 mL). The combined organic layers were dried (MgSO₄) and concentrated in vacuo. The residue was chromatographed (PE/EA=8:1) to give 12 (500 mg, 95%) as a white solid. LCMS: BONB-00111-141-1 (EST) m/z=234.1 (M-Tert-butyl)⁺, t=3.337 min (215 nm).

Step 2

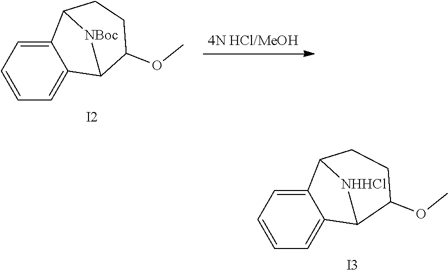

### 9-Methoxy-12-azatricyclo[6.3.1.0^{2,7}]dodeca-2,4,6-triene hydrochloride (13)

To a stirred solution of compound 12 (0.54 g, 1.87 mmol) in MeOH (5 mL) was added 4N HCl/MeOH (5 mL) at RT. The mixture was stirred at RT for 2 h. TLC showed compound 12 was consumed. Then the reaction was concentrated to give crude of 9-methoxy-12-azatricyclo[6.3.1.0^{2,7}]dodeca-2,4,6-triene hydrochloride (0.35 g). It was used in next step directly without further purification.

Step 3

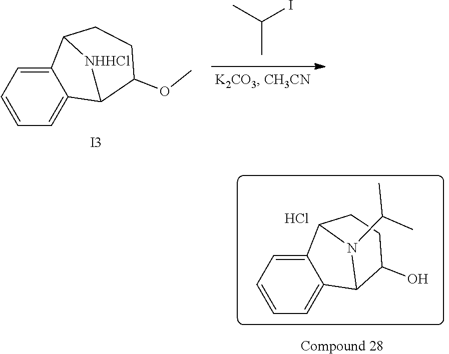

### 9-methoxy-12-(propan-2-yl)-12-azatricyclo[6.3.1.0^{2,7}]dodeca-2,4,6-triene hydrochloride (Compound 28)

To a stirred solution of compound 13 (200 mg, 0.87 mmol) and potassium carbonate (367 mg, 2.66 mmol) in ACN (15 mL) was added 2-iodopropane (0.2 ML, 2.66 mmol) at RT. The mixture was stirred at 50° C. for 8 h in a sealed tube. LCMS showed the reaction was completed. The reaction was concentrated and extracted with EtOAc (50 mL*2). The combined organic layers were dried over anhydrous Na₂SO₄, filtered and concentrated. The residue obtained was purified by prep-HPLC (base) and acidified by 2 drops of 4N HCl/MeOH to give Compound 28 (120 mg, 51%) as a light yellow solid. ¹H NMR (400 MHz, D₂O) δ 7.51-7.34 (m, 4H), 5.21-4.99 (m, 2H), 4.08-3.86 (m, 1H), 3.47 (d, J=9.6 Hz, 3H), 3.24 (dt, J=13.1, 6.5 Hz, 1H), 2.19-2.00 (m, 1H), 1.98-1.75 (m, 2H), 1.50-1.20 (m, 6H), 0.62 (ddd, J=24.2, 13.0, 6.8 Hz, 1H). LCMS: CP-0023226-022-P (ESI) m/z=232.1 (M+H)⁺, t=2.985 min (215 nm).

Compound 30 and Compound 35

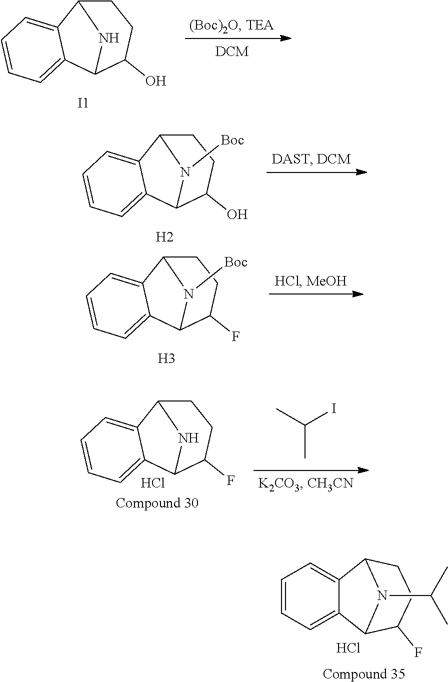

Step 1

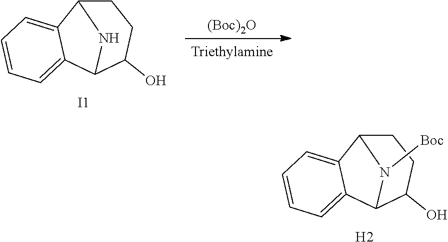

### tert-Butyl 9-hydroxy-12-azatricyclo[6.3.1.0^{2,7}]dodeca-2,4,6-triene-12-carboxylate (H2)

12-Azatricyclo[6.3.1.0^{2,7}]dodeca-2,4,6-trien-9-ol (1 g, 5.70 mmol) was dissolved in methylene chloride (100 mL). Di-tert-butyl dicarbonate (1.86 g, 8.55 mmol) and triethylamine (1.73 g, 17.1 mmol) were added and the mixture was stirred at room temperature overnight, LC-MS show that the reactant was consumed. The mixture was diluted by water (100 mL) and extracted with DCM (100 mL×3). The combined organic layers were dried over Na₂SO₄, filtered and concentrated. The crude product was chromatographed on silica gel to afford H2 (1.10 g, 70%) as a colorless solid. LCMS: BONB-00122-072-12671-6120-02 (ESI) m/z=220, 1 (M-O(CCH₃)₃)⁺, t=2.901 min (215 nm)

Step 2

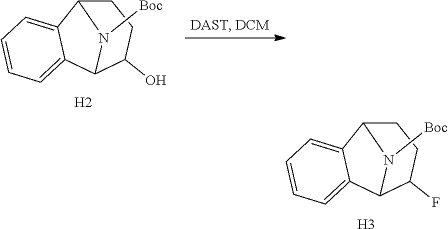

### tert-Butyl 9-fluoro-12-azatricyclo[6.3.1.0^{2,7}]dodeca-2,4,6-triene-12-carboxylate (H3)

tert-Butyl 9-hydroxy-12-azatricyclo[6.3.1.0^{2,7}]dodeca-2,4,6-triene-12-carboxylate (500 mg, 1.81 mmol) was dissolved in methylene chloride (50 mL). Diethyl(trifluoro-lambda4-sulfanyl)amine (2.91 g, 18.1 mmol) was added at −65° C. under N₂. The mixture was allowed to warm to room temperature for 5 h. The reaction mixture was quenched by the addition of saturated aqueous NH4C1 and extracted with DCM (30 mL×3). The organic layer was dried over MgSO₄, filtered and concentrated. The crude product was chromatographed on silica gel to afford 3 (300 mg, 59%) as a colorless solid. LCMS. BONB-00122-073-12861-6120-02(ESI) m/z=222.1 (M-O(CCH₃)₃)⁺, t=3.402 min (215 nm).

Step 3

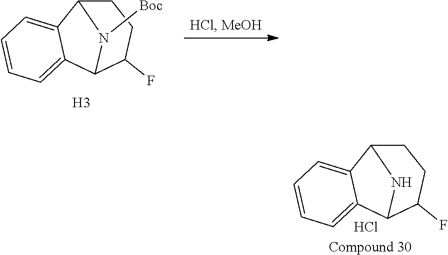

### 9-Fluoro-12-azatricyclo[6.3.1.0^{2,7}]dodeca-2,4,6-triene hydrochloride (Compound 30)

tert-Butyl 9-fluoro-12-azatricyclo[6.3.1.0^{2,7}]dodeca-2,4,6-triene-12-carboxylate (400 mg, 1.44 mmol) was dissolved in a solution of 1M HCl in methanol (50 mL). The mixture was stirred at room temperature for 18 h. The reaction solution was concentrated and the residue was washed with Et₂O (20 mL) to afford Compound 30 (100 mg, 39%) as a white solid. ¹H NMR (400 MHz, D₂O) δ 7.57 (t, J=6.2 Hz, 1H), 7.52-7.37 (m, 2H), 7.26 (d, J=7.4 Hz, 1H), 6.25-5.95 (m, 1H), 5.09-4.99 (m, 1H), 4.91 (d, J=5.7 Hz, 1H), 4.55 (t, J=6.5 Hz, 1H), 2.41-2.25 (m, 2H), 2.24-2.11 (m, 1H), 2.10-1.84 (m, 2H). LCMS: BONB-00122-078(ESI) m/z=178.1 (M+H)⁺, t=2.592 min (215 nm).

Step 4

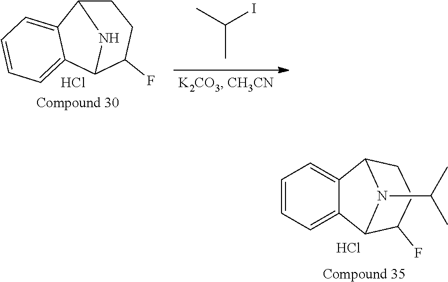

### 9-Fluoro-12-(propan-2-yl)-12-azatricyclo[6.3.1.0^{2,7}]dodeca-2,4,6-triene hydrochloride (Compound 35)

9-Fluoro-12-azatricyclo[6.3.1.0^{2,7}]dodeca-2,4,6-triene hydrochloride (300 mg, 1.41 mmol) was dissolved in acetonitrile (10 mL). Potassium carbonate (971 mg, 7.04 mmol) and 2-iodopropane (1.2 g, 7.04 mmol) were added, and the reaction mixture was stirred at 50° C. overnight. LC-MS show that the reactant was consumed. The mixture was filtered and the filtrate was concentrated. The residue was purified by prep-HPLC to give Compound 35 (38 mg, 11%) as a white solid. ¹H NMR (400 MHz, DMSO) δ 7.49 (s, 4H), 6.37-6.05 (m, 1H), 5.13 (d, J=31.7 Hz, 1H), 4.67 (d, J=52.4 Hz, 1H), 3.38 (d, J=7.0 Hz, 1H), 2.24 (dt, J=58.9, 15.8 Hz, 2H), 1.89 (d, J=17.2 Hz, 1H), 1.42 (dd, J=22.4, 6.2 Hz, 4H), 1.27 (d, J=5.5 Hz, 2H), 1.09 (t, J=7.0 Hz, 1H). LCMS: BONB-00125-054-P (ESI) m/z=220.1 (M+H)⁺, t=1.419 min (214 nm).

Compound 32 and Compound 33

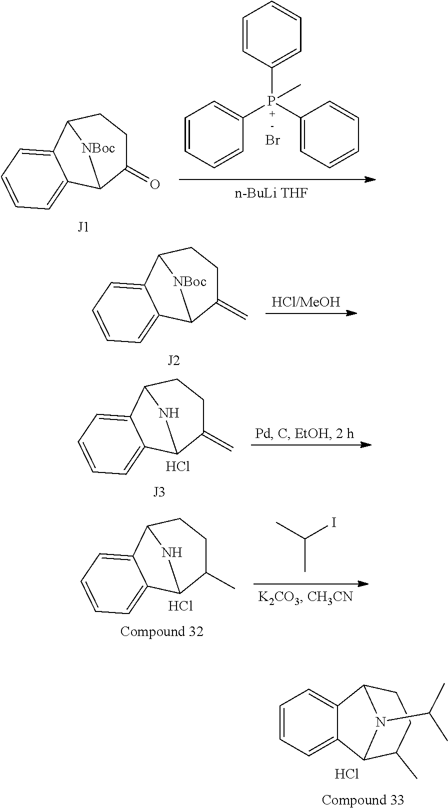

Step 1

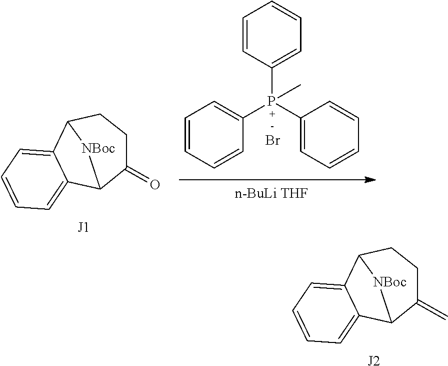

### tert-Butyl 9-methylidene-12-azatricyclo[6.3.1.0^{2,7}]dodeca-2,4,6-triene-12-carboxylate (J2)

To a stirred solution of methyltriphenylphosphonium bromide (3.9 g, 10.9 mmol) in THF (40 mL) was added at 0° C. butyllithium (678 mg, 10.9 mmol) in hexane. After 25 min, a solution of tert-butyl 9-oxo-12-azatricyclo[6.3.1.0^{2,7}]dodeca-2,4,6-triene-12-carboxylate (2 g, 7.1 mmol) in THF (6 mL) was added. The reaction mixture was heated to reflux for 18 h. The mixture was poured into saturated aqueous NH4C1 (40 mL). The aqueous layer was extracted with DCM (3×40 mL). The combined organic layers were dried over Na₂SO₄, filtered and concentrated in vacuo. The residue was purified by column chromatography (PE/EA=25/1) to give J2 (1.7 g, 88%) as a yellow oil. LCMS: BONB-00125-105-P (ESI) m/z=271.9 (M+H)⁺, t=2.887 min (215 nm).

Step 2

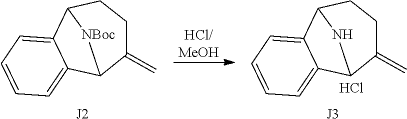

### 9-Methylidene-12-azatricyclo[6.3.1.0^{2,7}]dodeca-2,4,6-triene hydrochloride (J3)

tert-Butyl-9-methylidene-12-azatricyclo[6.3.1.0^{2,7}]dodeca-2,4,6-triene-12-carboxylate (1.7 g, 6.27 mmol) was dissolved in MeOH (5 mL), HCl/MeOH (5 mL, 4 M) was added and the mixture was stirred at 25° C. for 16 h. The mixture was concentrated in vacuo and washed by Et₂O (20 mL). The residue was dried in vacuo to give J3 (1.25 g, 96%) as a white solid. LCMS: BONB-00125-108-2(ESI) m/z=172.0 (M+H)⁺, t=1.977 min (215 nm).

Step 3

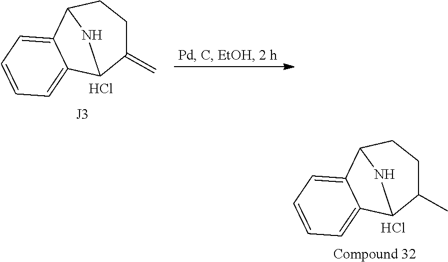

### 9-Methyl-12-azatricyclo[6.3.1.0^{2,7}]dodeca-2,4,6-triene hydrochloride (Compound 32)

9-Methylidene-12-azatricyclo[6.3.1.0^{2,7}]dodeca-2,4,6-triene hydrochloride (1.25 g, 6.04 mmol) was dissolved in ethyl alcohol (50 mL). Pd/C (5%, 0.25 g) was added, and the reaction mixture was stirred at room temperature for 2 hour under H2 atmosphere. LC-MS show that 3 was consumed. The mixture was filtered and the filtrate was concentrated to give the crude product. The crude product was acidified with HCl/MeOH and washed by Et₂O (20 mL). The residue was dried in vacuo to give Compound 32 (1.2 g, 95%) as a white solid. ¹H NMR (400 MHz, D₂O) δ 7.52-7.23 (m, 4H), 4.93 (t, J=2.7 Hz, 1H), 4.66 (d, J=2.6 Hz. 1H), 2.22 (dddd, J=14.6, 12.0, 7.4, 4.9 Hz, 1H), 2.05 (dddd, J=15.2, 12.5, 5.4, 2.9 Hz, 1H), 1.78 (dd, J=14.9, 5.9 Hz. 1H), 1.52 (dt, J=14.1, 5.1 Hz, 1H), 0.84 (d, J=6.8 Hz, 3H), 0.46 (ddd, J=26.5, 12.3, 6.0 Hz, 1H). LCMS: BONB-00122-097-1(ESI) m/z=293.9 (M+H)⁺, t=2.418 min (215 nm).

Step 4

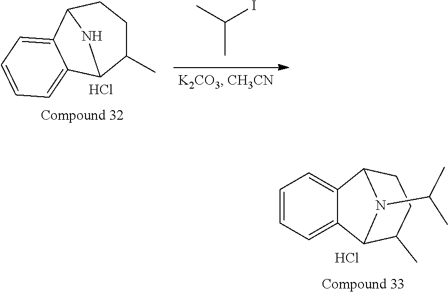

### 9-Methyl-12-(propan-2-yl)-12-azatricyclo[6.3.1.0^{2,7}]dodeca-2,4,6-triene hydrochloride (Compound 33)

9-Methylidene-12-azatricyclo[6.3.1.0^{2,7}]dodeca-2,4,6-triene hydrochloride (200 mg, 0.95 mmol) was dissolved in acetonitrile (20 mL). Potassium carbonate (657 mg, 4.78 mmol) and 2-iodopropane (0.8 g, 4.78 mmol) were added, and the reaction mixture was stirred at 50° C. overnight. LC-MS show that the reactant was consumed. The mixture was filtered and the filtrate was concentrated. The residue was purified by HPLC to give Compound 33 (66 mg, 27%) as a white solid. ¹H NMR (400 MHz, D₂O) δ 7.49-7.34 (m, 4H), 5.12 (s, 1H), 4.86 (d, J=2.8 Hz, 1H), 3.11 (s, 1H), 2.36-2.18 (m, 1H), 2.15-2.01 (m, 1H), 1.96-1.81 (m, 1H), 1.58-1.45 (m, 1H), 1.38-1.18 (m, 6H), 0.85 (dd, J=12.9, 6.7 Hz, 3H), 0.45 (tt, J=23.2, 8.5 Hz, 1H), LCMS: BONB-00125-037(ESI) m/z=216.0 (M+H)⁺, t=2.958 min (215 nm).

Compound 34

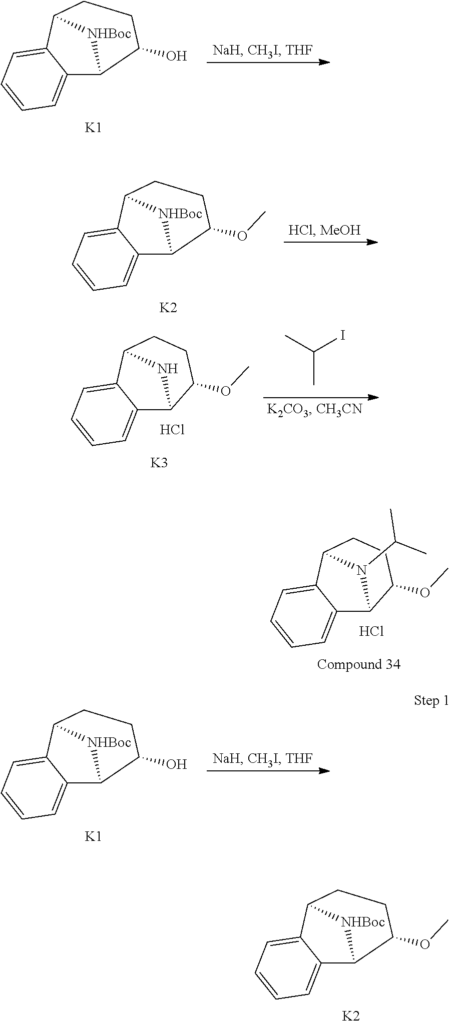

### tert-Butyl 9-methoxy-12-azatricyclo[6.3.1.0^{2,7}]dodeca-2,4,6-triene-12-carboxylate (K2)

tert-Butyl-9-hydroxy-12-azatricyclo[6.3.1.0^{2,7}]dodeca-2,4,6-triene-12-carboxylate (300 mg, 1.08 mmol) was dissolved in tetrahydrofuran (5 mL). Sodium hydride (300 mg, 12.5 mmol) was added at 0° C., and the reaction mixture was stirred at room temperature for 1.5 h. Iodomethane (167 mg, 1.18 mmol) was added, and the mixture was stirred at room temperature for 2 h. The reaction was poured into ice-water, extracted with EA (10 mL×3), dried over Na₂SO₄, filtered and concentrated to give K2 (210 mg, 67%) as yellow oil. LCMS: BONB-00121-059S2 (ESI) m/z=189.9 (M+H)⁺, t=2.334 min (215 nm).

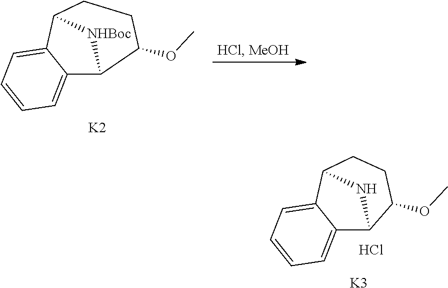

### 9-Methoxy-12-azatricyclo[6.3.1.0^{2,7}]dodeca-2,4,6-triene hydrochloride (K3)

tert-Butyl 9-methoxy-12-azatricyclo [6.3.1.0^{2,7}]dodeca-2,4,6-triene-12-carboxylate (420 mg, 1.45 mmol) was dissolved in 4N HCl in MeOH (5 mL). The reaction mixture was stirred at room temperature overnight. The mixture was concentrated in vacuo and washed with Et₂O (20 mL). The organic layers were concentrated to dryness and the residue was dried in vacuo to give K3 (110 mg, 40%) as a white solid. ¹H NMR (400 MHz, CDCl₃) δ 7.26-7.14 (m, 4H), 4.34 (d, J=10.2 Hz, 2H), 3.44 (d, J=3.9 Hz, 3H), 2.08-1.99 (m, 1H), 1.58 (dd, J=14.9, 5.6 Hz, 1H), 1.41 (dd, J=13.3, 6.4 Hz, 1H), 1.27 (d, J=11.1 Hz, 1H), 1.17-1.07 (m, 1H).

Step 3

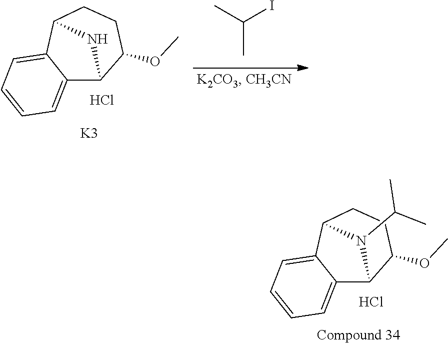

### 9-Methoxy-12-(propan-2-yl)-12-azatricyclo[6.3.1.0^{2,7}]dodeca-2,4,6-triene hydrochloride (Compound 34)

9-Methoxy-12-azatricyclo[6.3.1.0^{2,7}]dodeca-2,4,6-triene (90 mg, 475 μmol) was dissolved in acetonitrile (3 mL). 2-Iodopropane (88.7 mg, 522 μmol) and potassium carbonate (656 mg, 4.75 mmol) were added, and the reaction was stirred at RT for 48 h. LC/MS showed a very small amount of start material. The mixture was diluted with water, and extracted with DCM (10 mL×3), the organic layer was dried over Na₂SO₄, filtered and concentrated. The residue was purified by prep-HPLC to give Compound 34 (80.0 mg, 73%) as a white solid. ¹H NMR (400 MHz, D₂O) δ 7.49-7.27 (m, 4H), 5.18 (d, J=2.8 Hz, 1H), 5.08 (s, 1H), 3.78 (s, 1H), 3.37 (s, 3H), 3.22-3.08 (m, 1H), 2.22 (s, 1H), 1.74 (dd, J=15.1, 4.3 Hz, 1H), 1.58 (dd, J=15.4, 4.8 Hz, 1H), 1.17 (dd, J=33.2, 6.5 Hz, 6H), 1.00 (s, 1H). LCMS: BONB-00121-061 (ESI) m/z=231.9 (M+H)⁺, t=2.571 min (215 nm).

Compound 29

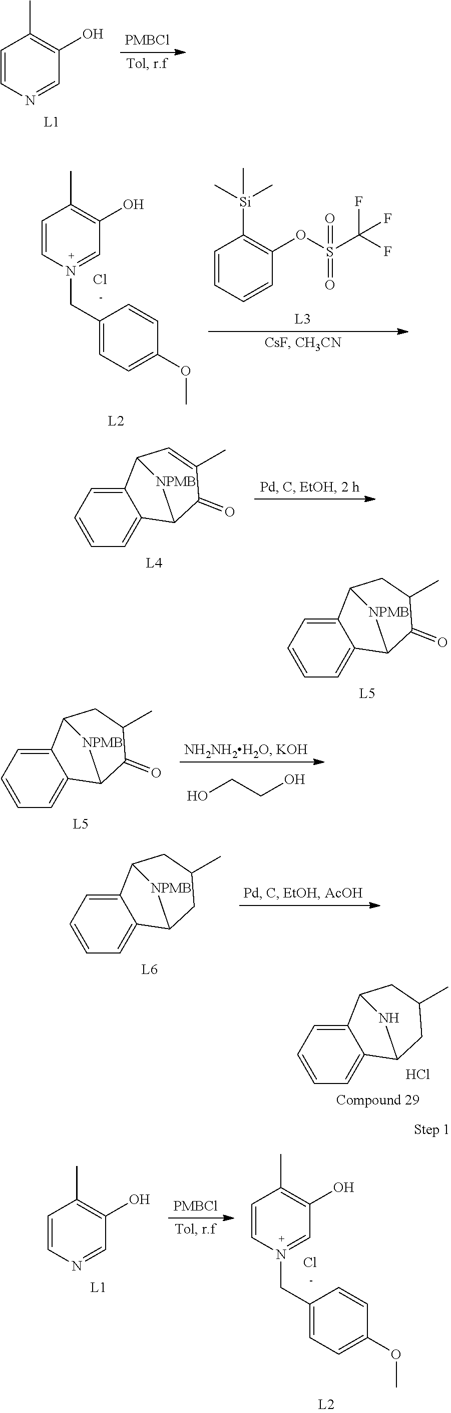

### 3-Hydroxy-1-(4-methoxybenzyl)-4-methylpyridin-1-ium chloride (L2)

4-Methylpyridin-3-ol (5 g, 45 mmol) was dissolved in toluene (100 mL). 1-(Chloromethyl)-4-methoxybenzene (7.2 g, 45 mmol) was added and the reaction mixture was stirred at 120° C. for overnight. The solvent was evaporated and the residue was washed with PE and filtered. The filter was dried over in vacuo to give 3-hydroxy-1-(4-methoxybenzyl)-4-methylpyridin-1-ium chloride (9 g, 76%) as a brown solid. LCMS: BONB-00125-019(ESI) m/z=229.9 (M+H)⁺, t=2.094 min (215 nm).

Step 2

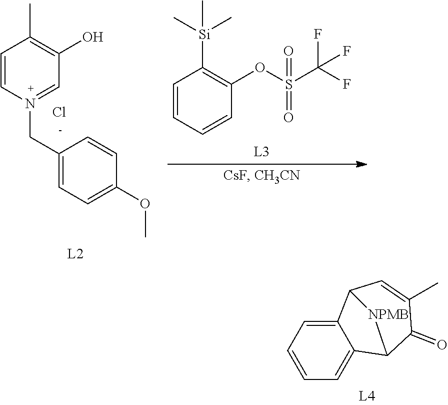

### 12-[(4-methoxyphenyl)methyl]-10-methyl-12-azatricyclo[6.3.1.0^{2,7}]dodeca-2,4,6,10-tetraen-9-one (L4)

3-hydroxy-1-(4-methoxybenzyl)-4-methylpyridin-1-ium chloride (9 g, 34 mmol) was dissolved in acetonitrile (300 mL), 2-(trimethylsilyl)phenyltrifluoromethanesulfonate (17.4 g, 58.4 mmol) and cesium fluoride (35.3 g, 233 mmol) were added and the mixture was stirred at 25° C. for 18 h. The mixture was filtered and the filtrate was concentrated under reduced pressure. The residue was purified by column chromatography (petroleum ether-EtOAc=20/l) to afford L4 (5.8 g, 56%) as a brown oil. LCMS: BONB-00125-021-1(ESI) m/z=306.1 (M+H)⁺, t=3.208 min (215 nm).

Step 3

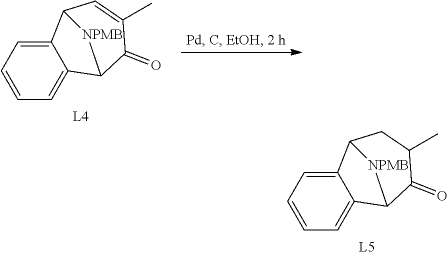

### 12-[(4-Methoxyphenyl)methyl]-10-methyl-12-azatricyclo[6.3.1.0^{2,7}]dodeca-2,4,6-trien-9-one (L5)

12-[(4-Methoxyphenyl)methyl]-10-methyl-12-azatricyclo[6.3.1.0^{2,7}]dodeca-2,4,6,10-tetraen-9-one (5.8 g, 18.9 mmol) was dissolved in ethyl alcohol (50 mL). Pd/C (5%, 0.87 g) was added, and the reaction mixture was stirred at RT under H2 atmosphere for 2 hours. LC-MS show that 4 was consumed. The mixture was filtered and the filtrate was concentrated to give the crude product. The crude product was purified by chromatography on silica gel (PE:EA=10:1) to give 5 (5.2 g, 90%) as an oil. LCMS. BONB-00125-025-1(ESI) m/z=308.1 (M+H)⁺, t=3.355 min (215 nm).

Step 4

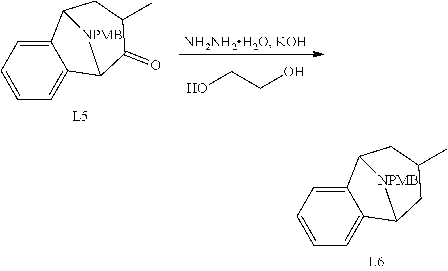

### 12-[(4-Methoxyphenyl)methyl]-10-methyl-12-azatricyclo[6.3.1.0^{2,7}]dodeca-2,4,6-triene (L6)

12-[(4-Methoxyphenyl)methyl]-10-methyl-12-azatricyclo[6.3.1.0²′7]dodeca-2,4,6-trien-9-one (5.2 g, 16.9 mmol) was dissolved in ethylene glycol (50 mL). Potassium hydroxide (9.42 g, 169 mmol) and hydrazine hydrate (3.35 g, 84.5 mmol) were added, and the reaction mixture was stirred at 190° C. for 4 hour. The mixture was cooled and diluted by water (150 mL). The mixture was extracted with DCM (100 mL×3). The combined organic layers were dried over Na₂SO₄, filtered and concentrated. The residue was purified by column chromatography on silica gel, eluting with (PE:EtOAc=20:1) to give 6 (3.5 g, 71%) as an oil. LCMS: BONB-00125-026 (ESI) m/z=294.1 (M+H)⁺, t=3.707 min and 3.707 min (215 nm), m/z=294.1 (M+H)⁺, t=3.850 min (215 nm).

Step 5

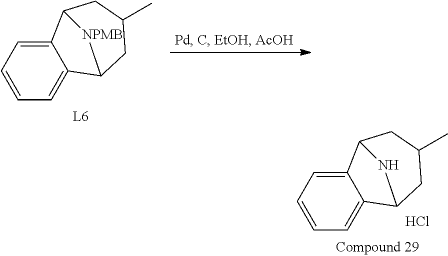

### 10-Methyl-12-azatricyclo[6.3.1.0^{2,7}]dodeca-2,4,6-triene hydrochloride (Compound 29)

12-[(4-Methoxyphenyl)methyl]-10-methyl-12-azatricyclo[6.3.1.0^{2,7}]dodeca-2,4,6-triene (3.5 g, 11.9 mmol) was dissolved in ethyl alcohol (40 mL). Acetic acid (36%, 2 mL) and Pd/C (5%, 3.5 g) were added, and the reaction mixture was stirred overnight at 40° C. under H₂ atmosphere. LC-MS show that L6 was consumed. The mixture was filtered and the filtrate was concentrated to give the crude product. The crude product was acidified with HCl/MeOH and washed by Et₂O (20 mL). The residue was dried in vacuo to give Compound 29 (1.7 g, 68%) as a white solid. LCMS: BONB-00125-030(ESI) m/z=174.1 (M+H)⁺, t=2.331 min (215 nm). ¹H NMR (400 MHz, D₂O) δ 7.30 (d, J=6.2 Hz, 4H), 4.86 (s, 2H), 1.84 (ddd, J=14.8, 5.1, 2.8 Hz, 2H), 1.69-1.48 (m, 2H), 1.06 (d, J=6.0 Hz. 1H), 0.73 (d, J=6.7 Hz, 3H).

Compound 31

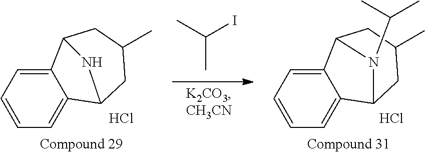

### 10-Methyl-12-(propan-2-yl)-12-azatricyclo[6.3.1.0^{2,7}]dodeca-2,4,6-triene hydrochloride (Compound 31)

10-Methyl-12-azatricyclo[6.3.1.0^{2,7}]dodeca-2,4,6-triene hydrochloride (350 mg, 1.67 mmol) was dissolved in acetonitrile (20 mL). Potassium carbonate (1.15 g, 8.37 mmol) and 2-iodopropane (1.4 g, 8.37 mmol) were added, and the reaction mixture was stirred at 50° C. overnight. LC-MS show that the reactant was consumed. The mixture was filtered and the filtrate was concentrated. The residue was purified by prep-HPLC to give Compound 31 (83 mg, 20%) as a white solid. ¹H NMR (400 MHz, D₂O) δ 7.47-7.31 (m, 4H), 5.12 (s, 2H), 3.09 (s, 1H), 2.17-1.61 (m, 5H), 1.30 (dd, J=47.5, 6.5 Hz, 6H), 0.81 (d, J=6.6 Hz, 3H). LCMS: BONB-00125-032(ESI) m/z=216.1 (M+H)⁺, t=2.579 min (215 nm).

Compound 36

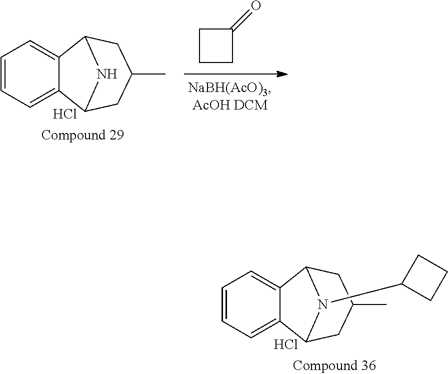

### 12-Cyclobutyl-10-methyl-12-azatricyclo[6.3.1.0^{2,7}]dodeca-2,4,6-triene hydrochloride (Compound 36)

10-Methyl-12-azatricyclo[6.3.1.0^{2,7}]dodeca-2,4,6-triene hydrochloride (300 mg, 1.43 mmol) was dissolved in DCM (10 mL). Cyclobutanone (501 mg, 7.15 mmol) and acetic acid (4.29 mg, 71.5 μmol) were added and the mixture was stirred at room temperature for 4 h. Sodium triacetoxyborohydride (1.66 g. 7.86 mmol) was added and the mixture continued stirring at rt overnight under N₂ atmosphere. The mixture was cooled and diluted by water (15 mL). The mixture was extracted with DCM (10 mL×3). The combined organic layers were dried over by Na₂SO₄, filtered and concentrated. The residue was purified by HPLC to give Compound 36 (120 mg, 37%). ¹H NMR (400 MHz, D₂O) δ 7.45 (ddd, J=26.2, 5.5, 3.2 Hz, 4H), 4.87 (s, 2H), 3.52 (d, J=8.4 Hz, 1H), 2.29-2.16 (m, 4H), 2.10-2.00 (m, 2H), 1.79 (s, 5H), 1.20 (ddt, J=17.7, 11.6, 5.9 Hz, 1H), 0.87 (d, J=6.7 Hz, 3H). LCMS: OAK-0011410-LCMS(ESI) m/z=228.0 (M+H)⁺, t=2.571 min (215 nm).

Compound 37

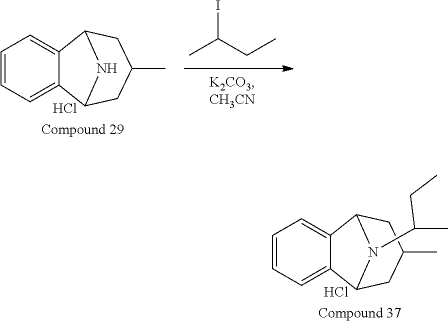

### 12-(Butan-2-yl)-10-methyl-12-azatricyclo[6.3.1.0^{2,7}]dodeca-2,4,6-triene hydrochloride (Compound 37)

10-Methyl-12-azatricyclo[6.3.1.0^{2,7}]dodeca-2,4,6-triene hydrochloride (220 mg, 1.04 mmol) was dissolved in CH₃CN (8 mL). 2-Iodobutane (1.14 g, 6.24 mmol) and K2CO₃ (898 mg, 8.32 mmol) were added and the mixture was stirred at 50° C. overnight. The mixture was filtered and the filtrate was concentrated under reduced pressure. The residue was purified by HPLC to give Compound 37 (150 mg, 62.34%). ¹H NMR (400 MHz, D₂O) δ 7.45 (d, J=17.0 Hz, 4H), 5.20 (s, 2H), 2.94 (s, 1H), 1.85 (s, 5H), 1.28 (d, J=6.5 Hz, 5H), 0.88 (dd, J=11.7, 6.7 Hz, 5H). LCMS: OAK-0011410-LCMS(ESI) m/z=230.1 (M+H)⁺, t=3.277 min (215 nm).

Compound 38

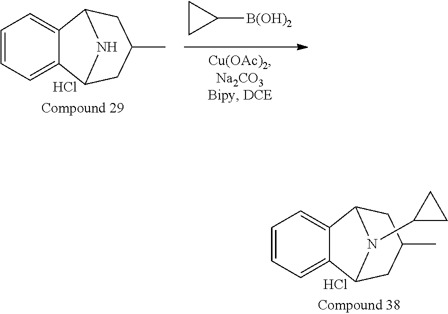

### 12-azatricyclo[6.3.1.0^{2,7}]dodeca-2,4,6-trien-10-yl}methylium hydrochloride (Compound 38)

12-cyclopropyl-12-azatricyclo[6.3.1.0^{2,7}]dodeca-2,4,6-triene hydrochloride (300 mg, 1.42 mmol) was dissolved in ethylene dichloride (15 mL). Cyclopropylboronic acid (241 mg, 2.84 mmol), 2,2′-bipyridine (210 mg, 1.42 mmol), Na₂CO₃ (300 mg, 3.30 mmol) and (acetyloxy)cuprio acetate (255 mg, 1.42 mmol) were added and the mixture was stirred at 70° C. overnight with reflux. The reaction solution was filtered and concentrated, the residue was purified by prep-HPLC to afford Compound 38 (32 mg, 9%) as a white solid. ¹H NMR (400 MHz, D₂O) δ 7.44 (d, J=2.4 Hz, 4H), 5.05 (s, 2H), 2.60 (s, 1H), 1.99 (d, J=14.0 Hz, 2H), 1.72 (t, J=12.4 Hz, 2H), 1.15 (dd, J=12.1, 6.0 Hz, 1H), 0.99 (s, 2H), 0.82 (t, J=16.1 Hz, 5H). LCMS: BONB-00125-079-LCMS(ESI) m/z=214.3 (M+H)⁺, t=2.873 min (215 nm).

Compound 39

Step 1

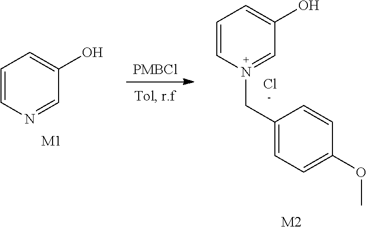

### 3-Hydroxy-1-[(4-methoxyphenyl)methyl]pyridin-1-ium chloride (M2)

Pyridin-3-ol (50 g. 0.52 mol) was dissolved in toluene (1 L), was added 1-(chloromethyl)-4-methoxybenzene (82.3 g, 0.52 mol) was added, and the reaction mixture was stirred at 120° C. overnight. The solvent was evaporated and the residue was washed with PE and filtered. The filter was dried in vacuo to give M2 (130 g, 98%) as a white solid. LCMS: BONB-00122-082 (EST) m/z=215.9 (M+H)⁺, t=2.095 min (215 nm).

Step 2

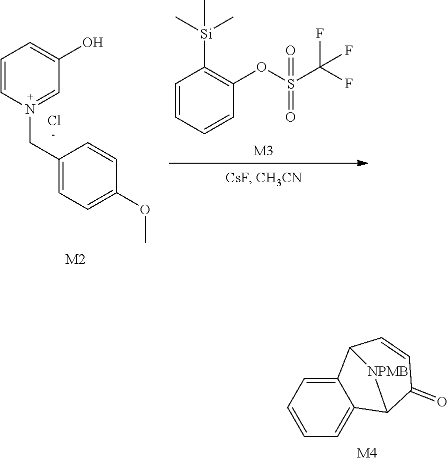

### 12-[(4-Methoxyphenyl)methyl]-12-azatricyclo[6.3.1.0^{2,7}]dodeca-2,4,6,10-tetraen-9-one (M4)

3-Hydroxy-1-[(4-methoxyphenyl)methyl]pyridin-1-ium chloride (50 g, 198 mmol) was dissolved in acetonitrile (300 mL). 2-(Trimethylsilyl)phenyltrifluoromethanesulfonate (88.6 g, 297 mmol) and cesium fluoride (179 g, 1.18 mol) were added, and the mixture was stirred at 25° C. for 18 h. The mixture was filtered and the filtrate was concentrated under reduced pressure. The residue was purified by column chromatography (petroleum ether-EtOAc) to afford 4 (38.7 g, 132 mmol, 66%) as a brown oil. LCMS: BONB-00122-089-1(ESI) m/z=291.9 (M+H)⁺, t=3.723 min (215 nm).

Step 3

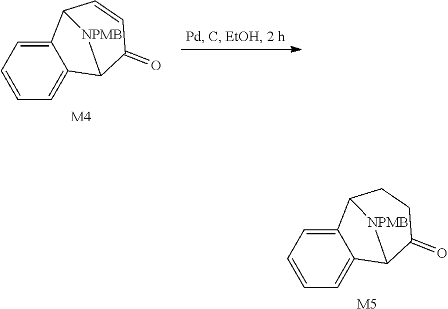

### 12-[(4-Methoxyphenyl)methyl]-12-azatricyclo[6.3.1.0^{2,7}]dodeca-2,4,6-trien-9-one (M5)

12-[(4-Methoxyphenyl)methyl]-12-azatricyclo[6.3.1.0^{2,7}]dodeca-2,4,6,10-tetraen-9-one (23 g, 10.6 mmol) was dissolved in ethyl alcohol (300 mL). Pd/C (5%, 3.5 g) was added, and the reaction mixture was stirred at RT under H2 atmosphere for 2 hours. LC-MS show that M4 was consumed. The mixture was filtered and the filtrate was concentrated to give the crude product. The crude product was purified by chromatography on silica gel (PE:EA=10:1) to give MS (21 g, 91%) as an oil. LCMS: BONB-00122-097-1(ESI) m/z=293.9 (M+H)⁺, t=3.172 min (215 nm).

Step 4

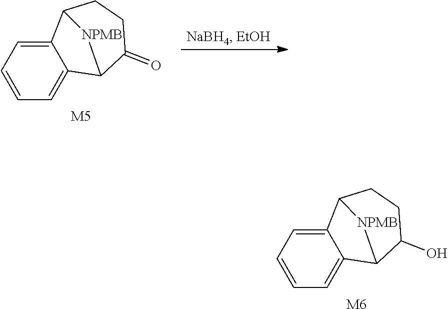

### 12-[(4-Methoxyphenyl)methyl]-12-azatricyclo[6.3.1.0^{2,7}]dodeca-2,4,6-trien-9-ol (M6)

To a stirred solution of compound MS (9.6 g, 32.73 mmol) in EtOH (100 mL) was slowly added sodium borohydride (1.85 g, 45.99 mmol) at 0° C. The reaction was stirred at 0° C. for 10 min, and warmed to RT. The mixture was stirred at RT for 1 h. TLC showed the compound 5 was consumed. The reaction was quenched with H₂O (20 mL). The mixture was concentrated and extracted with EtOAc (150 mL*2). The combined organic layers were dried over anhydrous Na₂SO₄, filtered and concentrated. The residue obtained was purified by column chromatography of silica gel eluting with petroleum ether/EtOAc from 100/1 to 5/1 to give M6 (6.6 g, 68%) as a light yellow solid.

Step 5

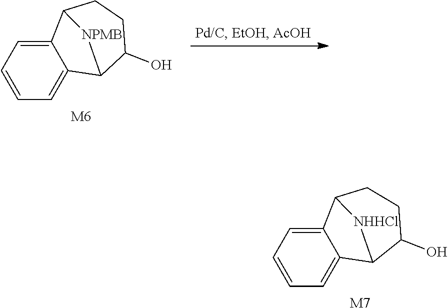

### 12-Azatricyclo[6.3.1.0^{2,7}]dodeca-2,4,6-trien-9-ol hydrochloride (M7)

To a stirred solution of 12-[(4-methoxyphenyl)methyl]-12-azatricyclo[6.3.1.0^{2,7}]dodeca-2,4,6-trien-9-ol (430 mg, 1.45 mmol) in ethyl alcohol (15 mL) was added acetic acid (0.4 mL) and 10% Pd/C (0.5 g) at RT. Then the mixture was hydrogenated at atmospheric pressure at 40° C. LCMS showed the compound M6 was consumed. The catalyst was filtered, rinsed with ethyl alcohol and vacuum evaporated. The residue was eluted with EtOAc (30 mL) and 4N HCl/EtOAc (5 mL). The mixture was stirred at RT for 10 min. The solid was filtered to give M7 (250 mg, 81%) as a white solid. ¹H NMR (400 MHz, D₂O) δ 7.42 (t, J=4.8 Hz, 4H), 4.98 (s, 1H), 4.80 (d, J=2.4 Hz, 1H), 4.15 (s, 1H), 2.39-2.23 (m, 11H), 1.71 (dd, J=14.0.4.0 Hz, 1H), 1.51 (dd, J=15.1, 4.0 Hz, 1H), 124-1.07 (m, 1H).

Step 6

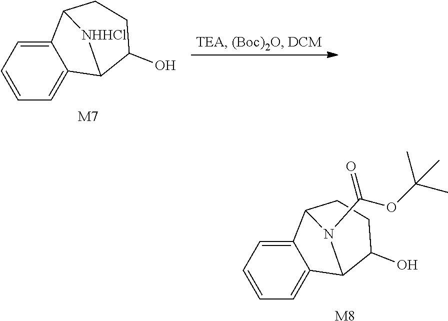

### Tert-butyl 9-hydroxy-12-azatricyclo[6.3.1.0^{2,7}]dodeca-2,4,6-triene-12-carboxylate (M8)

To a stirred solution of 12-azatricyclo[6.3.1.0^{2,7}]dodeca-2,4,6-trien-9-ol hydrochloride (0.2 g, 944 μmol) in methylene chloride (10 mL) was added triethylamine (286 mg, 2.83 mmol). The mixture was stirred at RT for 15 min. Then di-tert-butyl dicarbonate (307 mg, 1.41 mmol) was added to the mixture. The reaction was stirred at RT for 2 h. LCMS showed the reaction was completed. The reaction was extracted with DCM (50 mL) and water (50 mL). The organic layer was dried over anhydrous Na₂SO₄, filtered and concentrated. The residue obtained was purified by column chromatography of silica gel eluting with petroleum ether/EtOAc from 100/1 to 10/1 to give M8 (170 mg, 65%) as a white solid. ¹H NMR (400 MHz, CDCl₃) δ 7.29 (dd, J=8.3, 4.2 Hz, 2H), 7.25-7.15 (m, 1H), 5.19-4.83 (m, 2H), 3.99 (d, J=37.2 Hz, 1H), 2.08-1.71 (m, 3H), 1.57-1.43 (m, 9H), 1.35 (d, J=15.2 Hz, 1H), 0.64 (s, 1H).

Step 7

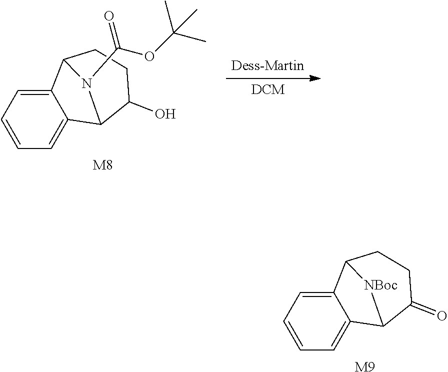

### Tert-butyl 9-oxo-12-azatricyclo[6.3.1.0^{2,7}]dodeca-2,4,6-triene-12-carboxylate (M9)

To a stirred solution of tert-butyl 9-hydroxy-12-azatricyclo[6.3.1.0^{2,7}]dodeca-2,4,6-triene-12-carboxylate (1.4 g, 5.08 mmol) in methylene chloride (30 mL) was added 1,1-bis(acetyloxy)-3-oxo-3H-1λ⁵,2-benziodaoxol-1-yl acetate (4.28 g, 10.1 mmol) at RT. The mixture was stirred at RT for 1.5 h. LCMS showed the reaction was complete. The reaction was quenched with aq. Na₂SO₃. The aqueous layer was further extracted with DCM (20 mL). The combined organic layers were dried over anhydrous Na₂SO₄, filtered and concentrated to give crude tert-butyl 9-oxo-12-azatricyclo[6.3.1.0^{2,7}]dodeca-2,4,6-triene-12-carboxylate (1.20 g, 86%) as a yellow oil. LCMS: BONB-00111-142-2-R (ESI) m/z=218.0 (M-Tert-butyl)⁺, t=3.166 min (215 nm).

Step 8

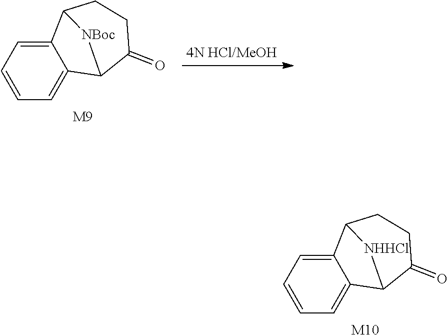

### 12-Azatricyclo[6.3.1.0^{2,7}]dodeca-2,4,6-trien-9-one hydrochloride (M10)

To a stirred solution of compound 9 (0.4 g) in MeOH (5 mL) was added 4N HCl/MeOH (5 mL) at RT. The mixture was stirred at RT for 2 h. TLC showed compound M9 was consumed. The reaction was concentrated to give crude 12-azatricyclo[6.3.1.0^{2,7}]dodeca-2,4,6-trien-9-one hydrochloride (0.26 g). It was used in next step directly.

Step 9

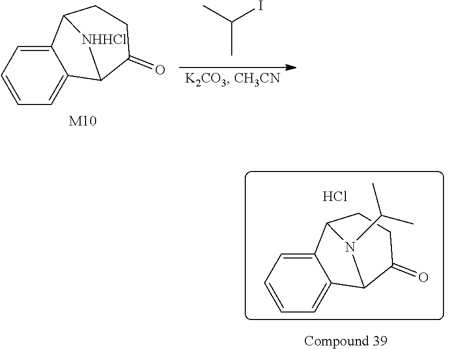

### 2-(propan-2-yl)-12-azatricyclo[6.3.1.0^{2,7}]dodeca-2,4,6-trien-9-one hydrochloride (Compound 39)

To a stirred solution of compound M10 (300 mg, 1.73 mmol) and potassium carbonate (727 mg, 5.19 mmol) in CH₃CN (15 mL) was added 2-iodopropane (0.4 mL, 5.19 mmol) at RT. The mixture was stirred at 50° C. for 8 h in a sealed tube. LCMS showed the reaction was completed. The reaction was concentrated and extracted with EtOAc (50 mL*2). The combined organic layers were dried over anhydrous Na₂SO₄, filtered and concentrated. The residue obtained was purified by prep-HPLC (base) and acidified by 2 drops of 4N HCl/MeOH to Compound 39 (90 mg, 24%) as a light yellow solid. ¹H NMR (400 MHz, D₂O) δ 7.43 (s, 4H), 5.18 (s, 1H), 3.23 (s, 1H), 2.25 (s, 1H), 1.90 (s, 1H), 1.72 (d, J=11.0 Hz, 1H), 1.24 (d, J=32.1 Hz, 6H), 1.04 (s, 1H). LCMS: BONB-00111-147-P2 (ESI) m/z=216.1 (M+H)⁺, t=1.534 min (215 nm).

Compound 40

Step 1

### (2-Bromo-4,5-difluorophenyl)trimethylsilane (N2)

To a solution of 2-bromo-4,5-difluorophenol (10 g, 47.8 mmol) in toluene (60 mL) was added hexamethyldisilizane (15.4 g, 95.6 mmol) at room temperature. The resulting mixture was refluxed for 3 h. After cooling to room temperature, the solvent and HMDS were removed under reduced pressure. The residue was used to the next step without further purification. ¹H NMR (400 MHz, CDCl₃): δ 7.84-7.80 (m, 1H), 7.16-7.15 (m, 1H), 0.30 (s, 9H).

Step 2

### 4,5-Difluoro-2-(trimethylsilyl)phenyl trifluoromethanesulfonate (N3)

To a stirred solution of (2-bromo-4,5-difluorophenyl)trimethylsilane (12.6 g, 47.5 mmol) in THF (30 mL) was added dropwise n-BuLi (19 mL, 2.5 M in hexanes) at −78° C. The reaction mixture was stirred for 20 min at −78° C. Tf₂O (19.9 g, 95.0 mmol) was added dropwise to the reaction mixture, and kept stirring for 20 min under −78° C. The reaction was quenched with cold saturated aqueous NaHCO₃ and the resulting mixture was extracted with EtOAc (100 mL*3). The combined organic extracts were dried over anhydrous Na₂SO₄, filtered and concentrated under reduced pressure. The residue was purified by silica gel (PE:EA=100:1) to afford N3 (8 g, Y: 50.6%) as colorless oil. ¹H NMR (400 MHz, CDCl₃): δ 7.32-7.21 (m, 2H), 0.36 (s, 9H). ¹H NMR: BONB-00112-159-1

Step 3

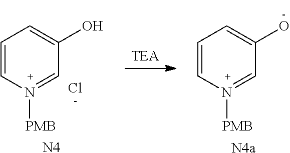

### 1-[(4-Methoxyphenyl)methyl]pyridin-1-ium-3-olate (N4a)

To a suspension of 3-hydroxy-1-[(4-methoxyphenyl)methyl]pyridin-1-ium chloride (6 g, 23.8 mmol) in CH₃CN (30 mL, dry) was added triethylamine (2.88 g, 28.5 mmol). The mixture was stirred at room temperature for 2 hours. The mixture was concentrated and dissolved in DCM and filtered. The filtrate was concentrated to give N4a (3.0 g, Y: 58.5%) without further purification.

LCMS: BONB-00112-164-01(ESI) m/z=216.2 (M+H)⁺, t=1.314 min (215 nm).

Step 4

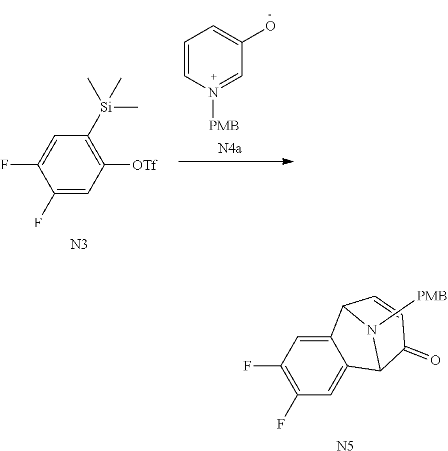

### 4,5-Difluoro-12-[(4-methoxyphenyl)methyl]-12-azatricyclo[6.3.1.0^{2,7}]dodeca-2,4,6,10-tetraen-9-one (N5)

3-Hydroxy-1-(4-methoxyphenyl)methylpyridin-1-ium chloride (1.4 g, 6.50 mmol) was dissolved in acetonitrile (50 mL). 4,5-Difluoro-2-(trimethylsilyl)phenyl trifluoromethanesulfonate (3.14 g, 9.42 mmol) and cesium fluoride (2.85 g, 18.8 mmol) were added, and the mixture was stirred at 25° C. for 3 h. The mixture was dissolved into EtOAc (100 mL), washed with water and brine. The organic layer was concentrated and purified by Prep-HPLC to give N5 (400 mg, 19.5%). LCMS: BONB-00112-168(ESI) m/z=328.0 (M+H)⁺, t=2.675 min (215 nm).

Step 5

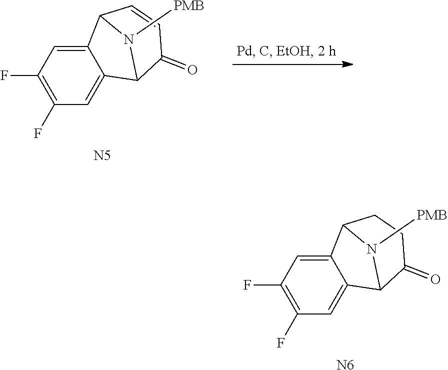

### 4,5-Difluoro-12-(4-methoxyphenyl)methyl-12-azatricyclo[6.3.1.0^{2,7}]dodeca-2,4,6-trien-9-one (N6)

4,5-Difluoro-12-[(4-methoxyphenyl)methyl]-12-azatricyclo[6.3.1.0^{2,7}]dodeca-2,4,6,10-tetraen-9-one (400 mg, 1.22 mmol) and palladium on activated carbon (5% Pd) (100 mg) were suspended in ethyl alcohol (30 mL) and the mixture was stirred at 25° C. for 2 hours under H2. LC-MS show that N5 was consumed. The reaction mixture was filtered through a celite cake and concentrated to give N6 as yellow oil (400 mg, Y: 99.7%). LCMS: BONB-00112-169(ESI) m/z=330.1 (M+H)⁺, t=1.540 min (215 nm).

Step 6

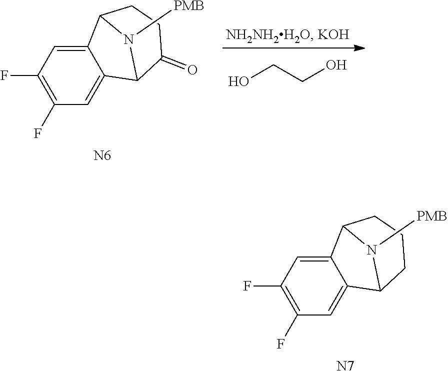

### 4,5-Difluoro-12-[(4-methoxyphenyl)methyl]-12-azatricyclo[6.3.1.0^{2,7}]dodeca-2,4,6-triene (N7)

4,5-Difluoro-12-[(4-methoxyphenyl)methyl]-12-azatricyclo[6.3.1.0^{2,7}]dodeca-2,4,6-trien-9-one (400 mg, 1.21 mmol) was dissolved in ethylene glycol (40 mL). Potassium hydroxide (339 mg, 6.05 mmol) and hydrazine hydrate (387 mg, 12.1 mmol) were added, and the reaction mixture was stirred at 190° C. for 4 hours. The mixture was cooled and diluted by water (30 mL). The mixture was extracted with DCM (50 mL×3). The combined organic layers were dried over Na₂SO₄, filtered and concentrated. The residue was purified by column chromatography on silica gel, eluting with (PE: EtOAc=20:1) to give 7 (250 mg, Y: 65.6%) as an oil. LCMS: BONB-00112-171(ESI) m/z=316.1 (M+H)⁺, t=1.631 min (215 nm).

Step 7

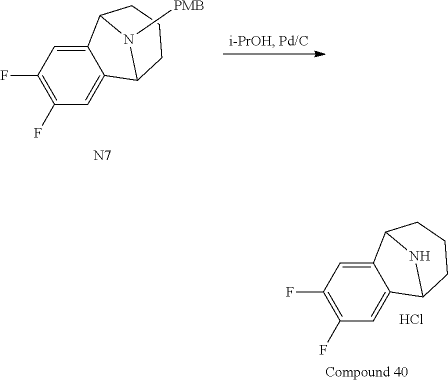

### 4,5-Difluoro-12-azatricyclo[6.3.1.0^{2,7}]dodeca-2,4,6-triene hydrochloride (Compound 40)

4,5-Difluoro-12-[(4-methoxyphenyl)methyl]-12-azatricyclo[6.3.1.0^{2,7}]dodeca-2,4,6-triene (200 mg, 634 μmol) and palladium on activated carbon (5% Pd) (220 mg) was dissolved in i-PrOH (15 mL) and the mixture was stirred at 40° C. for 2 hours under H2 atmosphere. LC-MS show that 7 was consumed. The reaction was filtered through a celite cake and concentrated to dryness. The crude material was purified by prep-HPLC. The eluent was acidified with HCl-dioxane (4M, 0.1 mL) and lyophilized overnight to give Compound 40 as a white solid (90 mg, 61.6%). ¹H NMR (400 MHz, D₂O): δ 7.34-7.09 (m, 2H), 4.94 (m, 0.78H), 4.80-4.79 (m, 0.73H), 4.38-4.37 (m, 0.61H), 4.39-4.34 (m, 0.66H), 2.93-2.89 (m, 0.65H), 2.33-2.27 (m, 1.35H), 2.17-2.07 (m, 1.48H), 1.87-1.80 (m, 1.50H), 1.50-0.96 (m, 1H). LCMS: BONB-00112-0178 (ESI) m/z=195.9 (M+H)⁺, t=2.578 min (215 nm).

### Compound 41 (4,5-Difluoro-12-(propan-2-yl)-12-azatricyclo[6.3.1.0^{2,7}]dodeca-2,4,6-triene)

4,5-Difluoro-12-azatricyclo[6.3.1.0^{2,7}]dodeca-2,4,6-triene

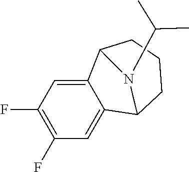 [SMILES: CC(C)N1C2CCCC1c1cc(F)c(F)cc12]

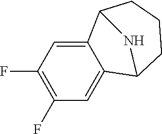 [SMILES: Fc1cc2c(cc1F)C1CCCC2N1]

(200 mg, 1.02 mmol) (Compound 40) was dissolved in acetonitrile (10 mL). 2-Iodopropane (520 mg, 3.06 mmol) and potassium carbonate (704 mg, 5.10 mmol) were added. The reaction mixture was stirred at 45° C. for 48 h, and the mixture was filtered and concentrated. The residue was purified by prep-HPLC, the eluent was acidified with HCl-dioxane (4M, 0.1 mL) and lyophilized overnight to give Compound 41 (80.0 mg, Y:29%) as a white solid.

¹H NMR (400 MHz, D2O): δ 7.62-6.82 (m, 2H), 5.49-4.75 (m, 2H), 4.14-2.78 (m, 2H), 2.51-0.66 (m, 11H).

LCMS: (ESI) m/z=237.9 (M+H)⁺, t=2.286 min (215 nm).

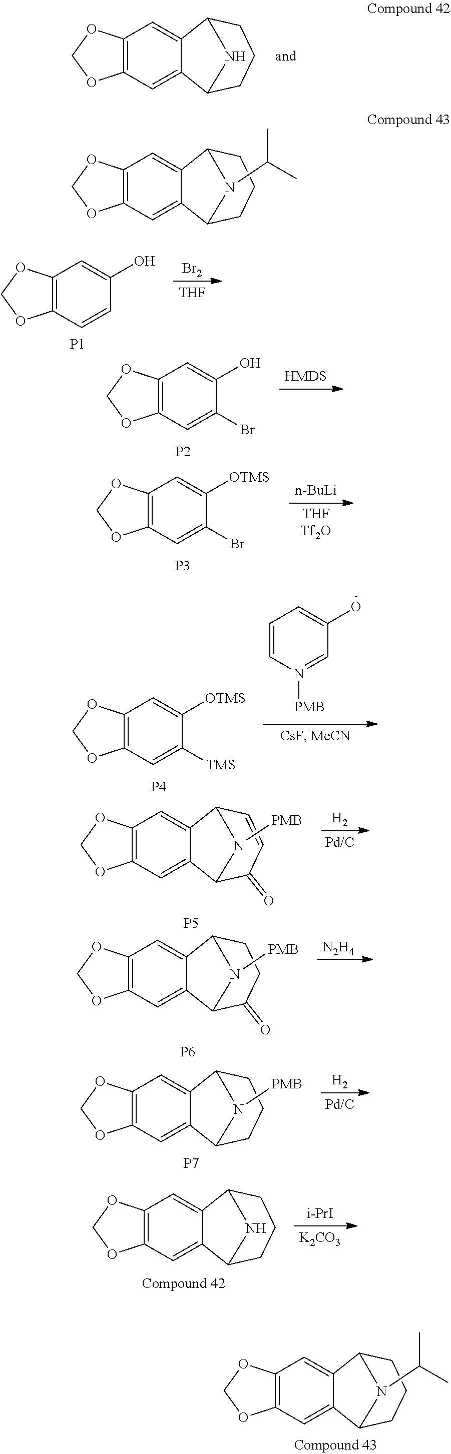

### 3-methoxy-11-azatricyclo[6.2.1.0^{2,7}]undeca-2,4,6,9-tetraene hydrochloride

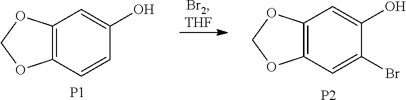

### 6-Bromo-2H-1,3-benzodioxol-5-ol (P2)

To a solution of benzo[d][1,3]dioxol-5-ol (100 g, 723 mmol) in THF (1 L) at 0° C., was added bromine (36.9 ml, 100 mmol). The reaction mixture was stirred at 0° C. for 25 min. The mixture was quenched by saturated Na₂S₂O₃-solution, and extracted with t-butyl methyl ether (300 mL×3). The combined organic layers were washed with brine, dried over anhydrous Na₂SO₄, filtered and concentrated to get crude product, which was purified by column chromatography (PE/EA=50:1) to get 6-bromo-2H-1,3-benzodioxol-5-ol (100 g, 64.1%) as colorless oil. ¹H NMR (400 MHz, DMSO) δ 9.82 (s, 1H), 7.03 (s, 1H), 6.59 (s, 1H), 5.95 (s, 2H).

### [(6-Bromo-2H-1,3-benzodioxol-5-yl)oxy]trimethylsilane (P3)

To a solution of 6-bromo-2H-1,3-benzodioxol-5-ol (100 g, 460 mmol) in THF (1.2 L) was added hexamethyldisilazane (144 mL, 690 mmol), and the mixture was refluxed with stirring for 3 h. The mixture was concentrated to get crude product [(6-bromo-2H-1,3-benzodioxol-5-yl)oxy]trimethylsilane (85 g, 63.9%) that was suitable for use without further purification. 1H NMR (400 MHz, CDCl₃) δ 6.83 (d, J=3.9 Hz, 1H), 6.33 (d, J=3.6 Hz, 1H), 5.81 (d, J=1.8 Hz, 2H), 0.17 (d, J=3.1 Hz. 9H).

### 6-(Trimethylsilyl)-2H-1,3-benzodioxol-5-yl trifluoromethanesulfonate (P4)

(6-Bromobenzo[d][1,3]dioxol-5-yloxy)trimethylsilane (80 g, 279 mmol) was dissolved in THF (800 mL), n-BuLi (2.5 M hexane solution, 121.2 mL, 303 mmol) was added at −78° C. and the mixture was stirred at −78° C. for 40 min. Trifluoromethanesulfonic anhydride (56 mL, 5.2 mmol) was added, and the mixture was stirred at −78° C. for 4 h. The reaction mixture was quenched with saturated NaHCO₃aq., and the mixture was slowly warmed to room temperature. The solution was extracted with Et₂O (200 mL×3), and the organic layer was washed with brine, dried over Na₂SO₄. After removal of the solvent, the residue was purified by column chromatography (PE/EA=20:1) to get 6-(trimethylsilyl)-2H-1,3-benzodioxol-5-yl trifluoromethanesulfonate (30 g, 31.7%). ¹H NMR (400 MHz, CDCl3) δ 6.87 (s, 1H), 6.84 (s, 1H), 6.03 (s, 2H), 0.33 (s, 9H).

### 15-[(4-Methoxyphenyl)methyl]-5,7-dioxa-15-azatetracyclo[9.3.1.0^{2,10}.0^{4,8}]pentadeca-2(10),3,8,13-tetraen-12-one (P5)

6-(Trimethylsilyl)-2H-1,3-benzodioxol-5-yl trifluoromethanesulfonate (30 g, 87.6 mmol) was dissolved in acetonitrile (400 mL). 1-[(4-Methoxyphenyl)methyl]pyridin-1-ium-3-olate (9.42 g, 43.8 mmol) and cesium fluoride (26.6 175.2 mmol) were added, and the mixture was stirred at 45° C. for 18 h. The mixture was filtered and the filtrate was concentrated under reduced pressure. The residue was purified by column chromatography (petroleum ether/EtOAc=5/1) to afford P5 (13 g, 44.3%) as a brown oil. LCMS: (ESI) m/z=335.9 (M+H)⁺, t=2.516 min (215 nm).

### 15-[(4-Methoxyphenyl)methyl]-5,7-dioxa-15-azatetracyclo[9.3.1.0^{2,10}.0^{4,8}]pentadeca-2,4(8),9-trien-12-one (P6)

15-[(4-Methoxyphenyl)methyl]-5,7-dioxa-15-azatetracyclo[9.3.1.0^{2,10}.0^{4,8}]pentadeca 2,4(8),9,13-tetraen-12-one (12 g, 35.7 mmol) was dissolved in ethyl alcohol (120 mL). Pd/C (2.4 g) was added, and the reaction mixture was stirred at room temperature under H2 atmosphere for 2 h. LC-MS show that compound P5 was consumed. The mixture was filtered and the filtrate was concentrated to give the crude product. The crude product was purified by CC (petroleum ether/EtOAc=10/1) to afford P6 (11 g, 91.6%) as yellow oil. LCMS: (ESI) m/z=337.9 (M+H)⁺, t=3.124 min (215 nm).

### 15-[(4-Methoxyphenyl)methyl]-5,7-dioxa-5-azatetracyclo[9.3.1.0^{2,10}.0^{4,8}]pentadeca-2,4(8),9-triene (P7)

15-[(4-Methoxyphenyl)methyl]-5,7-dioxa-15-azatetracyclo[9.3.1.0^{2,10}.0^{4,8}]pentadeca-2,4(8),9-trien-12-one (11 g, 32.6 mmol) was dissolved in ethylene glycol (150 mL). Potassium hydroxide (22.8 g, 163 mmol) and hydrazine hydrate (163 g, 326 mmol) were added, and the reaction mixture was stirred at 190° C. for 4 hour. The mixture was cooled and diluted with water (150 mL). The mixture was extracted with DCM (150 mL×3). The combined organic layers were dried over by Na₂SO₄, filtered and concentrated. The residue was purified by column chromatography on silica gel, eluting with (petroleum ether/EtOAc=4/1) to afford P7 (8 g, 40%) as an oil. LCMS: (EST) m/z=323.9 (M+H)⁺, t=3.181 min and 3.487 min (215 nm).

### 5,7-Dioxa-15-azatetracyclo[9.3.1.0^{2,10}0^{4,8}]pentadeca-2,4(8),9-triene (Compound 42)

15-[(4-Methoxyphenyl)methyl]-5,7-dioxa-15-azatetracyclo[9.3.1.0^{2,10}.0^{4,8}]pentadeca-2,4(8),9-triene (6 g, 18.5 mmol) was dissolved in ethyl alcohol (50 mL). Acetic acid (36% 1.5 mL) and Pd/C (6 g) were added, and the reaction mixture was stirred overnight at 40° C. under H2 atmosphere. LC-MS show that compound P7 was consumed. The mixture was filtered and the filtrate was concentrated to give the crude product. The crude product was purified by prep-HPLC to afford the title compound (2 g, 53.3%) as a colorless solid. LCMS: (ESI) m/z=202.0 (M+H)⁺, t=2.114 min (215 nm). 1H NMR (400 MHz, D₂O) δ 6.87 (s, 2H), 5.98 (d, J=14.9 Hz, 2H), 4.81 (s, 2H), 2.04-1.93 (m, 2H), 1.72 (d, J=12.4 Hz, 2H), 1.49 (d, J=13.9 Hz, 2H).

### 15-(Propan-2-yl)-5,7-dioxa-15-azatetracyclo[9.3.1.0^{2,10}.0^{4,8}]pentadeca-2,4(8),9-triene (Compound 43)

5,7-Dioxa-15-azatetracyclo[9.3.1.0^{2,10}.0^{4,8}]pentadeca-2,4(8),9-triene (500 mg, 2.46 mmol) was dissolved in acetonitrile (25 mL). Potassium carbonate (1.72 g, 12.3 mmol) and 2-iodopropane (4.18 g, 24.6 mmol) were added, and the reaction mixture was stirred at 50° C. overnight. LC-MS show that the reactant was consumed. The mixture was filtered and the filtrate was concentrated. The residue was purified by HPLC to afford the title compound (200 mg, 33.1%) as a colorless solid. LCMS: (ESI) m/z=245.9 (M+H)⁺, t=2.428 min (215 nm). 1H NMR (400 MHz, D₂O) δ 6.88 (s, 2H), 5.98 (d, J=11.0 Hz, 2H), 4.99 (s, 2H), 2.02 (d, J=12.9 Hz, 3H), 1.83 (d, J=13.1 Hz, 2H), 1.46 (s, 2H), 1.35 (d, J=6.4 Hz, 1H), 1.24 (d, J=6.5 Hz, 6H).

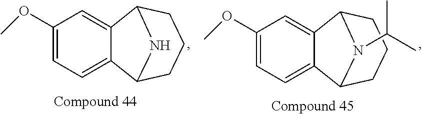

### Compound 48 and Compound 49

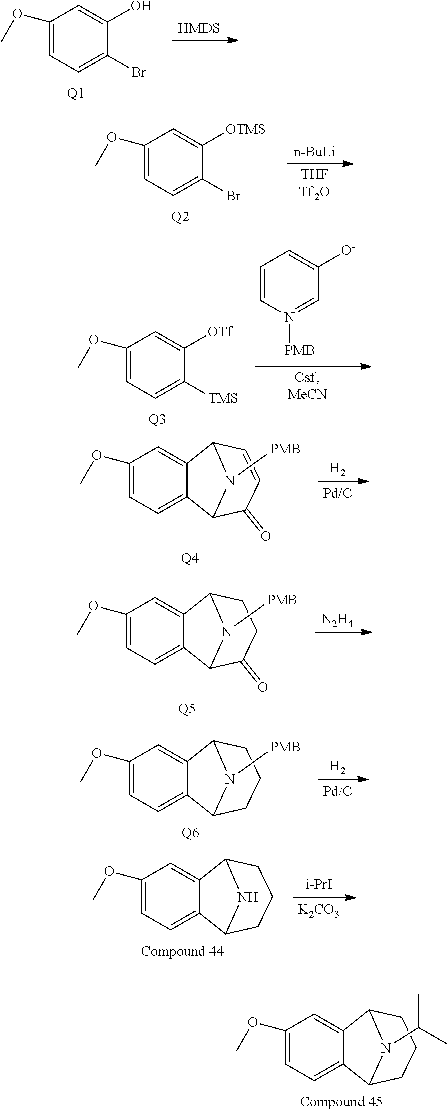

### (2-Bromo-5-methoxyphenoxy)trimethylsilane (Q2)

To a solution of 2-bromo-5-methoxyphenol (15 g, 73.88 mmol) in anhydrous THF (200 mL) was added bis(trimethylsilyl)amine (29.78 g, 184.5 mmol) under N₂ atmosphere. The reaction mixture was refluxed for 2 h. After cooling to room temperature, the crude product (2-bromo-5-methoxyphenoxy)trimethylsilane (20 g, 72.99 mmol, 98.8%) was attained by removing the solvent in vacuo and used for the next step without isolation. HNMR: 1H NMR (400 MHz, DMSO) δ 6.90 (d, J=2.9 Hz, 1H), 6.63 (dt, J=8.9, 5.9 Hz, 2H), 3.47 (s, 3H), 0.05-−0.06 (m, 9H).

### 5-Methoxy-2-(trimethylsilyl)phenyl trifluoromethanesulfonate (Q3)

The crude product 2-bromo-5-methoxyphenoxy)trimethylsilane (20 g. 72.99 mmol) was dissolved in anhydrous THF (300 mL) under N₂ atmosphere and cooled to −78° C. n-BuLi (32 mL, 1.1 equiv, 2.5 M in hexane) was added dropwise to the mixture and the reaction was stirred for 40 min at −78° C. Triflic anhydride (24.7 g, 1.2 equiv) was added to the mixture dropwise at −78° C. and the reaction was stirred for another 2 h. The reaction mixture was quenched with cold sat. NaHCO₃ aq at −78° C. and warmed to room temperature. The aqueous layer was extracted with Et₂O. The combined organic layers were dried over MgSO₄ and concentrated under reduced pressure. Finally, the residue was purified by silica gel column chromatography to afford 5-methoxy-2-(trimethylsilyl)phenyl trifluoromethanesulfonate (14 g, 42.7 mmol, 58.5%). ¹H NMR (400 MHz, CDCl3) δ 7.25 (d, J=9.7 Hz, 1H), 7.01 (d, J=3.2 Hz, 1H), 6.90 (dd, J=9.0, 3.2 Hz, 1H), 3.82 (s, 3H), 0.39-0.31 (m, 9H).

### 4-Methoxy-12-[(4-methoxyphenyl)methyl]-12-azatricyclo[6.3.1.0^{2,7}]dodeca-2,4,6,10-tetraen-9-one (Q4)

3-Hydroxy-1-[(4-methoxyphenyl)methyl]pyridin-1-ium chloride (6.15 g, 24.5 mmol) was dissolved in acetonitrile (100 mL). 5-Methoxy-2-(trimethylsilyl)phenyl trifluoromethanesulfonate (14 g, 42.7 mmol) and cesium fluoride (25.9 g, 170 mmol) were added, and the mixture was stirred at 25° C. bath for 18 h. The mixture was filtered and the filtrate was concentrated under reduced pressure. The residue was purified by column chromatography to afford 4-methoxy-12-[(4-methoxyphenyl)methyl]-12-azatricyclo[6.3.1.0²0.7]dodeca-2,4,6,10-tetraen-9-one (3.6 g, 11.2 mmol, 45.7%) as a brown oil. ¹H NMR (400 MHz, CDCl3) δ 7.27 (s, 1H), 7.21 (dd, J=11.4, 4.9 Hz, 3H), 7.03 (d, J=2.3 Hz, 1H), 6.90-6.84 (m, 3H), 6.69 (dd, J=8.1, 2.3 Hz, 1H), 5.53 (dd, J=9.8, 1.3 Hz, 1H), 4.34 (d, J=5.6 Hz, 2H), 3.80 (d, J=6.5 Hz, 6H), 3.66 (s, 2H).

### 4-Methoxy-12-[(4-methoxyphenyl)methyl]-12-azatricyclo[6.3.1.0^{2,7}]dodeca-2,4,6-trien-9-one (Q5)

4-Methoxy-12-[(4-methoxyphenyl)methyl]-12-azatricyclo[6.3.1.0^{2,7}]dodeca-2,4,6,10-tetraen-9-one (3.6 g, 11.2 mmol) was dissolved in methyl alcohol (30 mL). Pd/C (720 mg) was added, and the reaction mixture was stirred at RT under H2 atmosphere for 2 hours. LC-MS show that Q4 was consumed. The mixture was filtered and the filtrate was concentrated to give the crude product 4-methoxy-12-[(4-methoxyphenyl)methyl]-2-azatricyclo[6.3.1.0^{2,7}]dodeca-2,4,6-trien-9-one (3 g, 9.27 mmol, 83%) as oil. LCMS: (ESI) m/z=324.1 (M+H)⁺, t=2.430 min (220 nm).

### 4-Methoxy-12-[(4-methoxyphenyl)methyl]-12-azatricyclo[6.3.1.0^{2,7}]dodeca-2,4,6-triene (Q6)

4-Methoxy-12-[(4-methoxyphenyl)methyl]-12-azatricyclo[6.3.1.0^{2,7}]dodeca-2,4,6-trien-9-one (3 g, 9.27 mmol) was dissolved in ethylene glycol (50 mL). Potassium hydroxide (5.20 g, 92.7 mmol) and hydrazine hydrate (2.31 g, 46.3 mmol) were added, and the reaction mixture was stirred at 190° C. for 4 hour. The mixture was cooled and diluted with water (100 mL). The mixture was extracted with DCM (50 mL×3). The combined organic layers were dried over Na₂SO₄, filtered and concentrated. The residue was purified by column chromatography to give 4-methoxy-12-[(4-methoxyphenyl)methyl]-12-azatricyclo[6.3.1.0^{2,7}]dodeca-2,4,6-triene (1.3 g, 4.20 mmol, 45.4%) as an oil. LCMS: (EST) m/z=309.9 (M+H)⁺, t=3.434 min (215 nm).

### 4-Methoxy-12-azatricyclo[6.3.1.0^{2,7}]dodeca-2,4,6-triene hydrochloride (Compound 44)

4-Methoxy-12-[(4-methoxyphenyl)methyl]-12-azatricyclo[6.3.1.0^{2,7}]dodeca-2,4,6-triene (1.3 g, 4.20 mmol) was dissolved in methyl alcohol (20 mL). Acetic acid (36% 0.5 mL) and Pd/C (1.3 g) were added, and the reaction mixture was stirred overnight at 40° C. under H2 atmosphere. LC-MS show that Q6 was consumed. The mixture was filtered and the filtrate was concentrated to give the crude product. The crude product was acidified with HCL-MeOH and washed with Et₂O (20 mL). The residue was dried in vacuo to give the title compound (700 mg, 3.10 mmol, 73.8%) as a white solid. ¹H NMR (400 MHz, D₂O) δ 7.29 (d, J=8.1 Hz, 1H), 7.03-6.93 (m, 2H), 4.88 (d, J=2.5 Hz, 2H), 3.80 (s, 3H), 2.12-1.96 (m, 2H), 1.76 (s, 2H), 1.56-1.45 (m, 1H), 0.94 (dd, J=13.4, 7.1 Hz, 1H). LCMS: (ESI) m/z=190.0 (M+H)⁺, t=2.119 min (215 nm).

### 12-(Propan-2-yl)-12-azatricyclo[6.3.1.0^{2,7}]dodeca-2,4,6-triene (Compound 45)

4-Methoxy-12-azatricyclo[6.3.1.0^{2,7}]dodeca-2,4,6-triene hydrochloride (200 mg, 886 μmol) was dissolved in acetonitrile (10 mL). Potassium carbonate (612 mg, 4.43 mmol) and 2-iodopropane (753 mg, 4.43 mmol) were added, and the reaction mixture was stirred at 50° C. overnight. LC-MS show that the reactant was consumed, the reaction solution was filtered and concentrated, the residue was purified by prep-HPLC to afford the title compound (70 mg, 261 μmol, 29.5%) as a white solid. ¹H NMR (400 MHz, CDCl3) δ 7.27 (s, 1H), 7.21 (dd. J=11.4, 4.9 Hz, 3H), 7.03 (d, J=2.3 Hz, 1H), 6.90-6.84 (m, 3H), 6.69 (dd. J=8.1, 2.3 Hz, 1H), 5.53 (dd, J=9.8, 1.3 Hz, 1H), 4.34 (d, J=5.6 Hz, 2H), 3.80 (d, J=6.5 Hz, 6H), 3.66 (s, 2H). LCMS: (ESI) m/z=232.0 (M+H)⁺, t=2.046 min (215 nm).

Compound 45 is separated into Compound 48 and Compound 49 via any chiral separation method, such as chiral HPLC.

Compound 46 and Compound 47

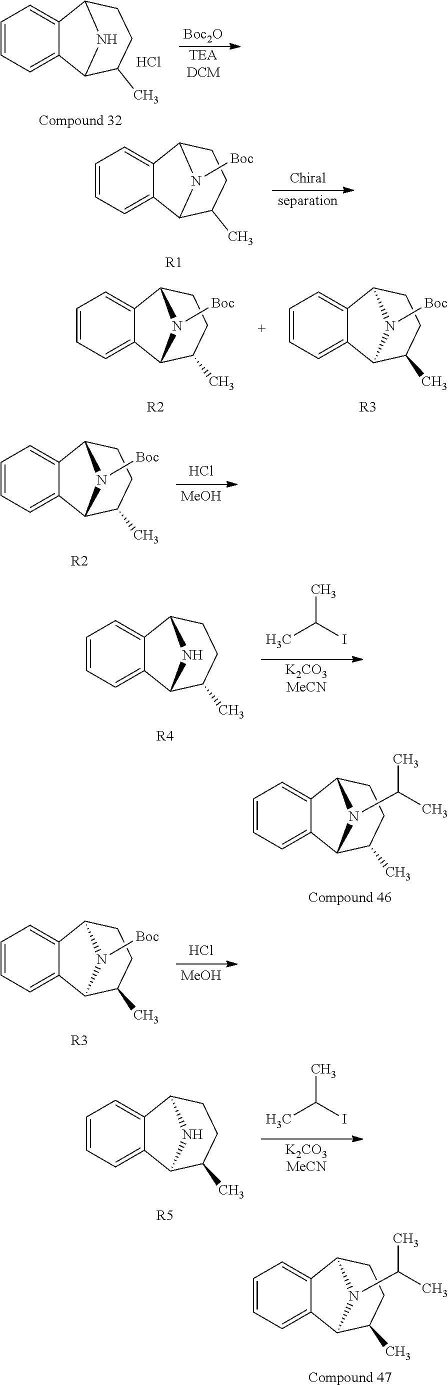

### tert-Butyl 9-methyl-12-azatricyclo[6.3.1.0^{2,7}]dodeca-2,4,6-triene-12-carboxylate (R2 and R3)

9-methyl-2-azatricyclo[6.3.1.0^{2,7}]dodeca-2,4,6-triene hydrochloride (1.3 g 6.19 mmol) was dissolved in methylene chloride (20 mL), was added triethylamine (1.87 g, 18.5 mmol). The mixture was stirred at RT for 15 min. Then di-tert-butyl dicarbonate (1.61 g, 7.42 mmol) was added to the mixture. The reaction was stirred at RT for 2 h. LCMS showed the compound 1 was consumed. The reaction was poured into water (50 mL), extracted with DCM (50 ml×3). The organic layer was dried over anhydrous Na2S4, filtered and concentrated. The residue was purified by column chromatography of silica gel eluting with with petroleum ether/EtOAc from 100/1 to 10/1 to give R1 (1.6 g, 94%) as a yellow oil. Compound R1 was chirally separated to give R12 (524 mg, 1.91 mmol) and R3 (411 mg, 1.50 mmol) as colourless oils.

LCMS: (EST) m/r=217.9 (M-Tert-butyl)⁺, t=2.925 min (215 nm).

### (9S)-9-methyl-12-azatricyclo[6.3.1.0^{2,7}]dodeca-2,4,6-triene hydrochloride (R4)

tert-Butyl (9S)-9-methyl-12-azatricyclo[6.3.1.0^{2,7}]dodeca-2,4,6-triene-12-carboxylate (524 mg, 1.91 mmol) was dissolved in MeOH (5 mL), was added 4N HCl/MeOH (5 mL). The mixture was stirred at RT for 2 h. TLC showed starting material was consumed. Then the reaction was concentrated and the residue was washed with Et2O to give R4 (350 mg, 87%). LCMS: (ESI) m/z=173.9 (M+H)⁺, t=1.854 min (215 nm).

### (1S,8R,9S)-9-methyl-12-(propan-2-yl)-12-azatricyclo[6.3.1.0^{2,7}]dodeca-2,4,6-triene hydrochloride (Compound 46)

(9S)-9-Methyl-12-azatricyclo[6.3.1.0^{2,7}]dodeca-2,4,6-triene hydrochloride (350 mg, 1.67 mmol) was dissolved in CH₃CN (15 mL), was added 2-iodopropane (1.4 g, 8.35 mmol) and K₂CO₃ (691 mg, 5.01 mmol). The mixture was stirred at 50° C. for 8 h in a sealed tube. LCMS showed the reaction was completed. The reaction was concentrated and extracted with EtOAc (50 mL*2). The combined organic layers were dried over anhydrous Na₂SO₄, filtered and concentrated. The residue was purified by prep-HPLC (base) and acidified by 4N HCl/MeOH to Compound 46 (240 mg, 57%) as a white solid.

1H NMR (4(0) MHz, D2O) δ 7.49-7.35 (m, 4H), 5.13 (t, J=2.7 Hz, 1H), 4.86 (d, J=2.7 Hz, 1H), 3.12 (dt, J=13.1, 6.6 Hz, 1H), 2.28 (dd, J=7.0, 4.2 Hz, 1H), 2.16-2.03 (m, 1H), 1.93-1.83 (m, 1H), 1.51 (dt, J=13.8, 5.2 Hz, 1H), 1.37 (d, J=6.5 Hz, 1H), 1.25 (dd, J=6.5, 2.8 Hz. 5H), 0.85 (dd, J=13.0, 6.7 Hz, 3H), 0.46 (dd, J=14.2, 6.0 Hz, 1H). LCMS: (ESI) m/z=215.9 (M+H)⁺, t=2.116 min (215 nm).

### (9R)-9-methyl-12-azatricyclo[6.3.1.0^{2,7}]dodeca-2,4,6-triene hydrochloride (R5)

tert-butyl (9R)-9-methyl-12-azatricyclo[6.3.1.0^{2,7}]dodeca-2,4,6-triene-12-carboxylate (411 mg. 1.51 mmol) was dissolved in MeOH (5 mL), was added 4N HCl/MeOH (5 mL). The mixture was stirred at RT for 2 h. TLC showed starting material was consumed. Then the reaction was concentrated and the residue was washed with Et2O to give R5 (300 mg, 94%). LCMS: (ESI) m/z=173.9 (M+H)⁺, t=1.844 min (215 nm).

### (1S,8R,9R)-9-Methyl-12-(propan-2-yl)-12-azatricyclo[6.3.1.0^{2,7}]dodeca-2,4,6-triene hydrochloride (Compound 47)

(9R)-9-Methyl-12-azatricyclo[6.3.1.0^{2,7}]dodeca-2,4,6-triene hydrochloride (300 mg, 1.43 mmol) was dissolved in CH₃CN (15 mL), was added K₂CO₃ (592 mg, 4.29 mmol) and 2-iodopropane (1.2 g, 7.15 mmol). The mixture was stirred at 50° C. for 8 h in a sealed tube. LCMS showed the reaction was completed. The reaction was concentrated and extracted with EtOAc (50 mL*2). The combined organic layers were dried over anhydrous Na₂SO₄, filtered and concentrated. The residue was purified by prep-HPLC (base) and acidified by 4N HCl/MeOH to Compound 47 (270 mg, 75%) as a white solid.

1H NMR (400 MHz, D₂O) δ 7.43 (ddd, J=22.7, 12.8, 6.8 Hz, 4H), 5.13 (s, 1H), 4.90 (d, J=29.2 Hz, 1H), 3.11 (dt, J=13.1, 6.6 Hz, 1H), 2.29 (s, 1H), 2.20-2.02 (m. 1H), 1.93-1.81 (m, 1H), 1.71-1.48 (m, 1H), 1.38 (d, J=6.4 Hz, 1H), 1.26 (d, J=6.3 Hz, 5H), 0.85 (dd, J=12.8, 6.7 Hz, 3H), 0.44 (ddd, J=26.4, 12.4, 6.1 Hz, 1H). LCMS: (EST) m/z=215.9 (M+H)⁺, t=2.295 min (215 nm).

### Example 2. In Vivo Neuropharmacological Assays

The compounds of the present disclosure were evaluated for their CNS related properties (e.g., the treatment, prevention or diagnosis of CNS or CNS-related disorders and/or amelioration of symptoms) using neuropharmacological screening methods described in S. L. Roberts et al., Front. Neurosci. 2011, 5:103 (hereinafter referred to as “Roberts,” the contents of which are incorporated herein by reference in their entirety).

The SmartCube® system described herein was utilized to compare the behavioral signature of a test compound to a database of behavioral signatures obtained using a large set of diverse reference compounds (including but not limited to antipsychotics, anxiolytics, antidepressants and bipolar disorder medicine), thereby predicting the neuropharmacological effects of a test compound by similarity to major classes of compounds. It produces an activity signature indicating the probability that the activity of the test compound at the administered dose matches a given class of neuropharmacological agents. The test compound is simultaneously compared against multiple classes of agents; thus, a separate probability is generated for each behavioral effect measured (such as but not limited to anxiolytic activity and analgesic activity).

The compounds of the present disclosure were dissolved in a mixture of Pharmasolve™ (N-methyl-2-pyrrolidone (NMP)) and were injected i.p. 15 min. before the behavioral test. For each compound, injections were administered at different dose levels (e.g., 0.3, 1, 3, 10 and 30 mg per kg (mpk)). A compound's minimal effective dose (MED; see Table 3) is a measure of the compound's potency. The MED was defined as the dose (in mpk) having 50% or more total activity in SmartCube®. For each behavioral effect measured, results at the MED are presented. CNS activities of the compounds were recorded (see Table 2). In Table 1, a key to the percent probabilities (probability ×100) for each behavioral effect (“X”) are defined provided, where LOQ is the limit of quantification.

TABLE 1
Percent Probability Ranges

| | |
|---|---|
| + | LOQ ≤ X < 5% |
| ++ | 5% ≤ X < 25% |
| +++ | 25% ≤ X < 50% |
| ++++ | 50% ≤ X < 100% |

TABLE 2
CNS activities of the compounds

| Compound | | | | | | | | | | | | |
|---|---|---|---|---|---|---|---|---|---|---|---|---|
| Number | AD | AX | SD | AP | MS | PS | CE | AG | XG | HA | SE | UN |
| 1 | ++ | ++ | + | + | + | + | +++ | ++ | + | + | ++ | + |
| 2 | ++ | + | + | + | + | + | + | ++ | + | + | + | +++ |
| 3 | +++ | ++ | ++ | + | + | + | + | ++ | + | + | ++ | ++ |
| 4 | +++ | ++ | + | + | + | ++ | + | ++ | + | + | + | + |
| 5 | ++ | ++ | + | ++ | + | + | + | + | + | + | + | +++ |
| 6 | +++ | ++ | + | + | + | + | + | ++ | + | + | + | + |
| 7 | ++ | ++ | + | + | + | ++ | + | +++ | + | + | + | + |
| 8 | ++++ | ++ | + | + | + | ++ | + | + | + | + | + | + |
| 9 | +++ | ++ | + | + | + | + | + | ++ | ++ | + | + | ++ |
| 10 | ++++ | ++ | + | + | + | ++ | + | ++ | + | ++ | + | + |
| 11 | + | ++ | + | + | + | + | ++ | ++ | + | + | + | +++ |
| 12 | ++ | ++ | + | + | + | + | + | + | + | + | + | +++ |
| 13 | +++ | ++ | + | + | + | ++ | + | + | + | + | + | + |
| 14 | ++ | ++ | + | + | + | + | + | +++ | + | + | + | + |
| 15 | ++++ | ++ | + | + | + | ++ | + | ++ | ++ | + | + | + |
| 16 | +++ | ++ | + | + | + | + | + | ++ | + | ++ | + | ++ |
| 17 | ++ | ++ | + | + | + | ++++ | + | + | + | + | + | + |
| 18 | ++++ | ++ | + | + | + | + | + | + | + | + | + | + |
| 19 | ++ | ++++ | + | + | + | + | + | + | + | + | + | + |
| 20 | + | + | + | + | + | + | + | + | + | + | + | + |
| 21 | +++ | ++ | + | + | + | + | + | ++ | + | + | + | + |
| 22 | ++ | + | + | + | + | + | + | + | + | + | + | ++++ |
| 23 | + | + | + | + | + | + | + | + | + | + | + | ++ |
| 24 | +++ | + | + | + | + | ++ | + | + | + | + | + | + |
| 25 | ++++ | + | + | + | + | ++ | + | + | + | + | + | + |
| 26 | ++++ | + | + | + | + | ++ | + | + | + | ++ | + | + |
| 27 | +++ | ++ | + | + | + | ++ | + | ++ | + | ++ | + | + |
| 28 | ++ | ++ | + | + | + | ++ | + | ++ | + | + | + | +++ |
| 29 | ++ | +++ | + | + | + | + | + | ++ | + | + | + | + |
| 30 | + | ++ | + | + | + | + | + | + | + | + | + | + |
| 31 | ++++ | + | + | + | + | ++ | + | + | + | ++ | + | + |
| 32 | + | + | + | + | + | ++ | + | + | + | ++++ | + | + |
| 33 | ++++ | + | + | + | + | + | + | + | + | + | + | + |
| 34 | ++ | ++ | + | + | + | + | + | + | + | + | + | ++ |
| 35 | + | ++ | + | + | + | + | + | ++ | + | + | + | + |
| 36 | ++++ | + | + | + | + | + | + | + | + | ++ | + | ++ |
| 37 | ++ | +++ | + | + | + | ++ | + | ++ | + | + | + | + |
| 38 | ++++ | + | + | + | + | + | ++ | + | + | ++ | + | + |
| 39 | + | + | + | + | + | + | + | + | + | + | + | + |
| 40 | ++ | ++ | + | + | + | ++ | + | ++ | + | + | + | + |
| 41 | ++ | ++ | + | ++ | + | + | ++ | ++ | + | + | + | +++ |

AD: antidepressant;
AX: anxiolytic;
SD: sedative hypnotic;
AP: antipsychotic;
MS: mood stabilizer;
CE: cognitive enhancer;
AG: analgesic;
XG: anxiogenic;
HA: hallucinogenic;
SE: side effects;
UN: uncharacterized CNS activity

TABLE 3
MED values of the compounds

| | Compound |
|---|---|
| MED | Number |
| ++ | 1 |
| +++ | 2 |
| ++ | 3 |
| +++ | 4 |
| ++ | 5 |
| ++ | 6 |
| ++ | 7 |
| ++ | 8 |
| ++ | 9 |
| +++ | 10 |
| ++ | 11 |
| +++ | 12 |
| ++ | 13 |
| ++ | 14 |
| ++ | 15 |
| ++ | 16 |
| ++ | 17 |
| ++ | 18 |
| ++ | 19 |
| + | 20 |
| ++++ | 21 |
| +++ | 22 |
| + | 23 |
| ++ | 24 |
| +++ | 25 |
| +++ | 26 |
| ++ | 27 |
| ++ | 28 |
| +++ | 29 |
| + | 30 |
| +++ | 31 |
| ++ | 32 |
| +++ | 33 |
| ++ | 34 |
| + | 35 |
| +++ | 36 |
| +++ | 37 |
| ++ | 38 |
| + | 39 |
| + | 40 |
| ++ | 41 |

TABLE 4
Dose range values

| | | |
|---|---|---|
| 30 mg/kg < | + | |
| 10 mg/kg ≤ | ++ | ≤30 mg/kg |
| 1 mg/kg ≤ | +++ | <10 mg/kg |
| 0.1 mg/kg ≤ | ++++ | <1 mg/kg |

### Equivalents and Scope

Those skilled in the art will recognize, or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient: any method of production: any method of use, etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

1. A compound of Formula (I): 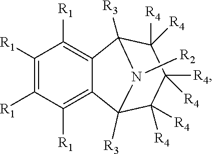
or a pharmaceutically acceptable salt thereof,
wherein R1 is independently H, optionally substituted alkyl, optionally substituted cycloalkyl, halogen, hydroxyl, alkoxyl, ether, CN, amine, aryl, or heteroaryl; wherein optionally any two adjacent R1 groups, together with the carbons to which they are attached, form a 5 to 8-membered carbocycle or heterocycle;
R2 is H, optionally substituted lower alkyl, optionally substituted cycloalkyl, optionally substituted heterocyclic, or optionally substituted multicyclic group: wherein optionally R2 and any R4, together with the nitrogen and carbon to which they are attached, form a 5 to 8-membered heterocycle;
R3 is independently H, halogen, optionally substituted lower alkyl, or optionally substituted cycloalkyl; wherein optionally R3 and any adjacent R4, together with the carbons to which they are attached, form a 5 to 8-membered carbocycle or heterocycle; and
R4 is independently H, optionally substituted lower alkyl, optionally substituted cycloalkyl, halogen, alkoxyl, CN, amine, aryl, heteroaryl, or carbonyl; wherein optionally any two geminal R4 groups, together with the carbon to which they are attached, form a 3 to 8-membered carbocycle or heterocycle.

2. The compound of claim 1, wherein at least one R3 is H.

3. The compound of claim 1, wherein R2 is an optionally substituted alkyl group, a cycloalkyl group, a heterocyclic group or a multicyclic group.

4. The compound of claim 1, wherein the compound is selected from the group consisting of Compounds 1-38 and 40-49 or a pharmaceutically acceptable salt thereof.

5. The compound of claim 1, wherein the compound is Formula (II): 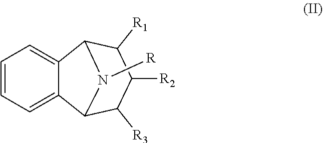
or a pharmaceutically acceptable salt thereof,
wherein R is hydrogen, an optionally substituted alkyl group, an optionally substituted cycloalkyl group, an optionally substituted heterocyclic group, or an optionally substituted multicyclic group; and
R1, R2, or R3, independently, is hydrogen, an optionally substituted alkyl group, an optionally substituted cycloalkyl group, a hydroxyl group, an alkoxy group, an aryloxy group, an amino group, or a halogen.

6. The compound of claim 5, wherein R is an optionally substituted alkyl group, an optionally substituted cycloalkyl group, an optionally substituted heterocyclic group or an optionally substituted multicyclic group.

7. The compound of claim 5, wherein R1, R2 and R3 are all hydrogens.

8. The compound of claim 7, wherein the compound is Compound 2, Compound 1, Compound 3, Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 23, Compound 24, Compound 25, Compound 26, or a pharmaceutically acceptable salt thereof.

9. The compound of claim 5, wherein at least one of R1, R2 or R3 is not hydrogen.

10. The compound of claim 9, wherein at least one of R1, R2 or R3 is —OH, —OCH3 or a halogen.

11. The compound of claim 9, wherein the compound is Compound 27, Compound 28, Compound 29, Compound 30, Compound 31, Compound 32, Compound 33, Compound 34, Compound 35, Compound 36, Compound 37, Compound 38, Compound 46, Compound 47, or a pharmaceutically acceptable salt thereof.

12. The compound of claim 1, wherein the compound is Formula (III): 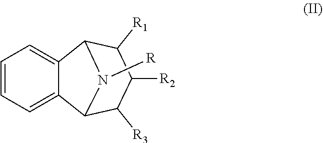
or a pharmaceutically acceptable salt thereof,
wherein R is hydrogen, an optionally substituted alkyl group, an optionally substituted cycloalkyl group, an optionally substituted heterocyclic group, or an optionally substituted multicyclic group;
R1, R2, or R3, independently, is an optionally substituted alkyl group, an optionally substituted cycloalkyl group, a hydroxyl group, an alkoxy group, an aryloxy group, an amino group, or a halogen, wherein any two adjacent R4 groups, together with the carbons to which they are attached, optionally form a 5 to 8-membered carbocycle or heterocycle which is optionally substituted; and
R4 or R5, independently, is H, lower alkyl, cycloalkyl, halogen, hydroxyl, alkoxyl, ether, CN, amine, aryl, or heteroaryl; wherein any two adjacent R4 groups, together with the carbons to which they are attached, optionally form a 5 to 8-membered carbocycle or heterocycle which is optionally substituted.

13. The compound of claim 12, wherein R is an optionally substituted alkyl group, an optionally substituted cycloalkyl group, an optionally substituted heterocyclic group or an optionally substituted multicyclic group.

14. The compound of claim 12, wherein R4 and R5 are both F.

15. The compound of claim 12, wherein the compound is Compound 40, Compound 41, Compound 42, Compound 43, Compound 44, Compound 45, Compound 48, Compound 49, or a pharmaceutically acceptable salt thereof.

16. A compound having a structure 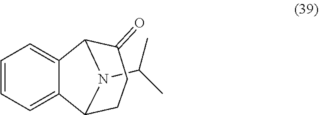 [SMILES: CC(C)N1C2CCC(=O)C1c1ccccc12]
or a pharmaceutically acceptable salt thereof.

17. A pharmaceutical composition comprising the compound of claim 1 or a pharmaceutically acceptable salt thereof, and at least one pharmaceutically acceptable excipient.

18. A method of treating, preventing, or managing a CNS disorder in a subject in need thereof, comprising administering to said subject an effective amount of the compound of claim 1.

19. The method of claim 18, wherein the CNS disorder is a neurological or psychiatric disorder.

20. The method of claim 18, wherein the CNS disorder is depression, anxiety, cognitive impairment, psychosis, schizophrenia, bipolar disorder, obsessive compulsive disorder (OCD), panic disorder, posttraumatic stress disorder (PTSD), addiction, social disorder, attention deficit hyperactivity disorder (ADHD), or autism.

21. The method of claim 20, wherein the CNS disorder is depression.

22. The method of claim 21, wherein the CNS disorder is bipolar depression, unipolar depression, major depressive disorder, treatment-resistant depression, suicidal behavior disorder, or anhedonia.
